(19) United States
(12) Patent Application Publication
(10) Pub. No.: US 2018/0244783 A1
(43) Pub. Date: Aug. 30, 2018

(54) COMBINATION THERAPY FOR TREATMENT OF DISEASE

(71) Applicant: OncoMed Pharmaceuticals, Inc., Redwood City, CA (US)

(72) Inventors: Timothy Charles HOEY, Hillsborough, CA (US); Christopher Lamond MURRIEL, San Francisco, CA (US); Jennifer Anne CAIN, Moss Beach, CA (US)

(21) Appl. No.: 15/755,142

(22) PCT Filed: Aug. 31, 2016
(86) PCT No.: PCT/US16/49703

§ 371 Date: Feb. 26, 2018

Related U.S. Application Data
(60) Provisional application No. 62/212,466, filed on Aug. 31, 2015.

(51) Int. Cl. C07K 16/28; A61P 35/00; A61K 38/17; A61K 39/395

(52) U.S. Cl. CPC C07K 16/2863; A61P 35/00; A61K 2039/507; C07K 16/2818; A61K 39/39558; A61K 38/179

(57) ABSTRACT

The present invention provides methods comprising combination therapy for modulating immune responses, for inhibiting tumor growth, and/or for treating cancer. In particular, the present invention provides Wnt pathway inhibitors in combination with immunotherapeutic agents for the treatment of cancer and other diseases.

### CROSS-REFERENCE TO RELATED APPLICATIONS

This application claims the priority benefit of U.S. Provisional Application No. 62/212,466, filed Aug. 31, 2015, which is hereby incorporated by reference herein in its entirety.

### FIELD OF THE INVENTION

The present invention provides methods comprising combination therapy for modulating immune responses and treating cancer and other diseases. In particular, the present invention provides Wnt pathway inhibitors in combination with at least one additional immunotherapeutic agent for the treatment of cancer.

### BACKGROUND OF THE INVENTION

Cancer is one of the leading causes of death in the developed world, with over one million people diagnosed with cancer and 500,000 deaths per year in the United States alone. Overall it is estimated that more than 1 in 3 people will develop some form of cancer during their lifetime. There are more than 200 different types of cancer, four of which—breast, lung, colorectal, and prostate—account for over half of all new cases (Siegel et al., 2012, CA: Cancer J. Clin., 62:10-29).

Signaling pathways normally connect extracellular signals to the nucleus leading to expression of genes that directly or indirectly control cell growth, differentiation, survival, and death. In a wide variety of cancers, signaling pathways are dysregulated and may be linked to tumor initiation and/or progression. Signaling pathways implicated in human oncogenesis include, but are not limited to, the Wnt pathway, the Ras-Raf-MEK-ERK or MAPK pathway, the PI3K-AKT pathway, the CDKN2A/CDK4 pathway, the Bcl-2/TP53 pathway, and the Notch pathway.

The Wnt signaling pathway has been identified as a potential target for cancer therapy. The Wnt signaling pathway is one of several critical regulators of embryonic pattern formation, post-embryonic tissue maintenance, and stem cell biology. More specifically, Wnt signaling plays an important role in the generation of cell polarity and cell fate specification including self-renewal by stem cell populations. Unregulated activation of the Wnt pathway is associated with numerous human cancers where it is believed the activation can alter the developmental fate of cells. The activation of the Wnt pathway may maintain tumor cells in an undifferentiated state and/or lead to uncontrolled proliferation. Thus carcinogenesis can proceed by overtaking homeostatic mechanisms which control normal development and tissue repair (reviewed in Reya & Clevers, 2005, Nature, 434:843-50; Beachy et al., 2004, Nature, 432:324-31).

The Wnt signaling pathway was first elucidated in the Drosophila developmental mutant wingless (wg) and from the murine proto-oncogene int-1, now Wnt1 (Nusse & Varmus, 1982, Cell, 31:99-109; Van Ooyen & Nusse, 1984, Cell, 39:233-40; Cabrera et al., 1987, Cell, 50:659-63; Rijsewijk et al., 1987, Cell, 50:649-57). Wnt genes encode secreted lipid-modified glycoproteins of which 19 have been identified in mammals. These secreted ligands activate a receptor complex consisting of a frizzled (FZD) receptor family member and low-density lipoprotein (LDL) receptor-related protein 5 or 6 (LRP5/6). The FZD receptors are seven transmembrane domain proteins of the G-protein coupled receptor (GPCR) superfamily and contain a large extracellular N-terminal ligand binding domain with 10 conserved cysteines, known as a cysteine-rich domain (CRD) or Fri domain. There are ten human FZD receptors, FZD1, FZD2, FZD3, FZD4, FZD5, FZD6, FZD7, FZD8, FZD9, and FZD10. Different FZD CRDs have different binding affinities for specific Wnt proteins (Wu & Nusse, 2002, J. Biol. Chem., 277:41762-9), and FZD receptors have been grouped into those that activate the canonical β-catenin pathway and those that activate non-canonical pathways (Miller et al., 1999, Oncogene, 18:7860-72).

A role for Wnt signaling in cancer was first uncovered with the identification of Wnt1 (originally intl) as an oncogene in mammary tumors transformed by the nearby insertion of a murine virus (Nusse & Varmus, 1982, Cell, 31:99-109). Additional evidence for the role of Wnt signaling in breast cancer has since accumulated. For instance, transgenic over-expression of β-catenin in the mammary glands results in hyperplasias and adenocarcinomas (Imbert et al., 2001, J. Cell Biol., 153:555-68; Michaelson & Leder, 2001, Oncogene, 20:5093-9) whereas loss of Wnt signaling disrupts normal mammary gland development (Tepera et al., 2003, J. Cell Sci., 116:1137-49; Hatsell et al., 2003, J. Mammary Gland Biol. Neoplasia, 8:145-58). In human breast cancer, β-catenin accumulation implicates activated Wnt signaling in over 50% of carcinomas, and though specific mutations have not been identified, up-regulation of FZD receptor expression has been observed (Brennan & Brown, 2004, J. Mammary Gland Biol. Neoplasia, 9:119-31; Malovanovic et al., 2004, Int. J. Oncol., 25:1337-42).

Activation of the Wnt pathway is also associated with colorectal cancer. Approximately 5-10% of all colorectal cancers are hereditary with one of the main forms being familial adenomatous polyposis (FAP), an autosomal dominant disease in which about 80% of affected individuals contain a germline mutation in the adenomatous polyposis coli (APC) gene. Mutations have also been identified in other Wnt pathway components including Axin and β-catenin. Individual adenomas are clonal outgrowths of epithelial cells containing a second inactivated allele, and the large number of FAP adenomas inevitably results in the development of adenocarcinomas through additional mutations in oncogenes and/or tumor suppressor genes. Furthermore, activation of the Wnt signaling pathway, including loss-of-function mutations in APC and stabilizing mutations in β-catenin, can induce hyperplastic development and tumor growth in mouse models (Oshima et al., 1997, Cancer Res., 57:1644-9; Harada et al., 1999, EMBO J., 18:5931-42).

Similar to breast cancer and colon cancer, melanoma often has constitutive activation of the Wnt pathway, as indicated by the nuclear accumulation of β-catenin. Activation of the Wnt/β-catenin pathway in some melanoma tumors and cell lines is due to modifications in pathway components, such as APC, ICAT, LEF1 and β-catenin (see e.g., Lame et al., 2006, Frontiers Biosci., 11:733-742). However, there are conflicting reports in the literature as to the exact role of Wnt/β-catenin signaling in melanoma. For example, one study found that elevated levels of nuclear β-catenin correlated with improved survival from melanoma, and that activated Wnt/β-catenin signaling was associated with decreased cell proliferation (Chien et al., 2009, PNAS, 106:1193-1198).

The basis for immunotherapy is the manipulation and/or modulation of the immune system, including both innate immune responses and adaptive immune responses. The general aim of immunotherapy is to treat diseases by controlling the immune response to a “foreign agent”, for example a pathogen or a tumor cell. However, in some instances immunotherapy is used to treat autoimmune diseases which may arise from an abnormal immune response against proteins, molecules, and/or tissues normally present in the body. Immunotherapy may include methods to induce or enhance specific immune responses or to inhibit or reduce specific immune responses. The immune system is a highly complex system made up of a great number of cell types, including but not limited to, T-cells, B-cells, natural killer cells, antigen-presenting cells, dendritic cells, monocytes, granulocytes, and macrophages. These cells possess complex and subtle systems for controlling their interactions and responses. The cells utilize both activating and inhibitory mechanisms and feedback loops to keep responses in check and not allow negative consequences of an uncontrolled immune response (e.g., autoimmune diseases).

Generally, an immune response is initiated through antigen recognition by the T-cell receptor (TCR) and is regulated by a balance between stimulatory and inhibitory signals (i.e., immune checkpoints). Under normal conditions, immune checkpoints are necessary to maintain a balance between activating and inhibitory signals and to ensure the development of an effective immune response while safeguarding against the development of autoimmunity or damage to tissues when the immune system is responding to a foreign or pathogenic agent. An important immune checkpoint receptor is CTLA-4 which is expressed on T-cells and is highly expressed on regulatory T-cells (Tregs). CTLA-4 is considered to act as an inhibitory molecule or an immune response “brake” and primarily regulates the amplitude of T-cell activation. CTLA-4 counteracts the activity of the co-stimulatory receptor, CD28, which acts in concert with the TCR to activate T-cells. CTLA-4 and CD28 share identical ligands or counter-receptors, B7-1 (CD80) and B7-2 (CD86) and the balance of the immune response probably involves competition of CTLA-4 and CD28 for binding to the ligands. Another important immune checkpoint receptor is PD-1 which is expressed on T-cells after activation, highly expressed on Tregs, and expressed on other activated cells including B-cells and natural killer (NK) cells. Similar to CTLA-4, PD-1 is considered to act as an inhibitory molecule and brake on the immune response. There are two ligands/counter-receptors for PD-1, PD-L1 (also known as B7-H1 and CD247) and PD-L2 (also known as B7-DC and CD273). (See, Pardoll, 2012, Nature Reviews Cancer, 12:252-264).

The concept of cancer immunosurveillance is based on the theory that the immune system can recognize tumor cells, mount an immune response, and suppress the development and/or progression of a tumor. However, it is clear that many cancerous cells have developed mechanisms to evade the immune system which can allow for uninhibited growth of tumors. Immune checkpoints can be dysregulated by tumors and may be manipulated by tumors to be used as an immune resistance mechanism. Cancer immunotherapy focuses on the development of agents that can activate and/or boost the immune system to achieve a more effective response to killing tumor cells and inhibiting tumor growth.

It is one of the objectives of the present invention to provide improved methods for cancer treatment, particularly methods using Wnt pathway inhibitors in combination with immunotherapeutic agents.

### BRIEF SUMMARY OF THE INVENTION

The present invention provides methods of treating diseases such as cancer, where the methods comprise administering to a subject in need thereof a FZD antagonist, such as an anti-FZD antibody or soluble FZD receptor, or another Wnt pathway inhibitor (e.g., small molecule) in combination with an immunotherapeutic agent. Combination therapy with at least two therapeutic agents can use agents that work by different mechanisms of action, and/or target different pathways and may result in additive or synergetic effects. Combination therapy may allow for a lower dose of each agent than used in monotherapy, thereby reducing toxic side effects and/or increasing the therapeutic index of the agent(s). Combination therapy may decrease the likelihood that resistance to an agent will develop. Combination therapy may allow one agent to sensitize tumor cells (including cancer stem cells) to enhanced activity by a second agent. Combination therapy comprising an immunotherapeutic agent may allow one agent to enhance the immune response to a tumor or tumor cells while the second agent may be effective at killing tumor cells more directly. In addition, the order and/or timing of the administration of each therapeutic agent may affect the overall efficacy of a drug combination.

The invention provides Wnt pathway inhibitors, including but not limited to, FZD antagonists. FZD antagonists include but are not limited to, antibodies and other polypeptides that bind to at least one FZD protein, small molecules that bind at least one FZD protein, and soluble FZD proteins. The FZD protein may be one of FZD1, FZD2, FZD3, FZD4, FZD5, FZD6, FZD7, FZD8, FZD9, and FZD10.

The invention provides immunotherapeutic agents, including but not limited to, a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

Compositions comprising a FZD antagonist or a different Wnt pathway inhibitor and/or at least one additional immunotherapeutic agent are provided. Pharmaceutical compositions comprising the Wnt pathway inhibitors and/or the immunotherapeutic agents are provided.

In one aspect, the invention provides methods of inhibiting tumor growth. In some embodiments, a method comprises contacting tumor cells with an effective amount of a Wnt pathway inhibitor in combination with an effective amount of an immunotherapeutic agent. The method may be in vivo or in vitro. In certain embodiments, the tumor is in a subject, and contacting tumor cells with the Wnt pathway inhibitor and the immunotherapeutic agent comprises administering a therapeutically effective amount of each of the agents to the subject. In some embodiments, a method of inhibiting tumor growth comprises administering to a subject a therapeutically effective amount of a Wnt pathway inhibitor and a therapeutically effective amount of an immunotherapeutic agent, where the Wnt pathway inhibitor is a FZD antagonist.

In another aspect, the invention provides a method of treating cancer. In some embodiments, a method of treating cancer comprises administering to a subject a therapeutically effective amount of a Wnt pathway inhibitor in combination with a therapeutically effective amount of an immunotherapeutic agent. In some embodiments, a method of treating cancer comprises administering to a subject a therapeutically effective amount of a Wnt pathway inhibitor and a therapeutically effective amount of an immunotherapeutic agent, where the Wnt pathway inhibitor is a FZD antagonist.

In another aspect, the invention provides a method of inhibiting the activity of regulatory T-cells (Tregs). In some embodiments, a method of inhibiting the activity of Tregs comprises administering to a subject a therapeutically effective amount of a Wnt pathway inhibitor in combination with a therapeutically effective amount of an immunotherapeutic agent. In some embodiments, a method of inhibiting the activity of Tregs comprises administering to a subject a therapeutically effective amount of a Wnt pathway inhibitor and a therapeutically effective amount of an immunotherapeutic agent, where the Wnt pathway inhibitor is a FZD antagonist. In some embodiments, the inhibition of Treg activity comprises inhibiting the suppression of immune responses. In some embodiments, the inhibition of Treg activity results in the inhibition of suppression of immune responses.

In another aspect, the invention provides a method of increasing T cell infiltration into a tumor. In some embodiments, a method of increasing T cell infiltration into a tumor comprises administering to a subject a therapeutically effective amount of a Wnt pathway inhibitor in combination with a therapeutically effective amount of an immunotherapeutic agent. In some embodiments, a method of increasing T cell infiltration into a tumor comprises administering to a subject a therapeutically effective amount of a Wnt pathway inhibitor and a therapeutically effective amount of an immunotherapeutic agent, where the Wnt pathway inhibitor is a FZD antagonist.

In another aspect, the invention provides a method of increasing T cell cytotoxicity to a tumor. In some embodiments, a method of increasing T cell cytotoxicity to a tumor comprises administering to a subject a therapeutically effective amount of a Wnt pathway inhibitor in combination with a therapeutically effective amount of an immunotherapeutic agent. In some embodiments, a method of increasing T cell cytotoxicity to a tumor comprises administering to a subject a therapeutically effective amount of a Wnt pathway inhibitor and a therapeutically effective amount of an immunotherapeutic agent, where the Wnt pathway inhibitor is a FZD antagonist.

In another aspect, the invention provides a method of increasing tumor cell lysis. In some embodiments, a method of increasing tumor cell lysis comprises administering to a subject a therapeutically effective amount of a Wnt pathway inhibitor in combination with a therapeutically effective amount of an immunotherapeutic agent. In some embodiments, a method of increasing tumor cell lysis which inhibits tumor relapse or tumor regrowth comprises administering to a subject a therapeutically effective amount of a Wnt pathway inhibitor and a therapeutically effective amount of an immunotherapeutic agent, where the Wnt pathway inhibitor is a FZD antagonist.

In another aspect, the invention provides a method to increase the efficacy of an immune checkpoint modulator. In some embodiments, a method to increase the efficacy of an immune checkpoint modulator comprises administering to a subject a therapeutically effective amount of a Wnt pathway inhibitor in combination with a therapeutically effective amount of an immune checkpoint modulator. In some embodiments, a method to increase the efficacy of an immune checkpoint modulator comprises administering to a subject a therapeutically effective amount of a Wnt pathway inhibitor and a therapeutically effective amount of an immune checkpoint modulator, where the Wnt pathway inhibitor is a FZD antagonist. In some embodiments, the immune checkpoint modulator is an immune checkpoint inhibitor. In some embodiments, the immune checkpoint modulator is an immune checkpoint enhancer or stimulator.

In another aspect, the invention provides a method of reducing or preventing metastasis in a subject. In some embodiments, a method of reducing or preventing metastasis in a subject comprises administering to the subject a therapeutically effective amount of a Wnt pathway inhibitor and a therapeutically effective amount of an immunotherapeutic agent. In some embodiments, a method of reducing or preventing metastasis in a subject comprises administering to the subject a therapeutically effective amount of a Wnt pathway inhibitor and a therapeutically effective amount of an immunotherapeutic agent, where the Wnt pathway inhibitor is a FZD antagonist.

In another aspect, the invention provides a method of enhancing treatment for a subject who is being treated with an immune checkpoint inhibitor, the method comprising administering to the subject a therapeutically effective amount of a Wnt pathway inhibitor, such as a FZD antagonist.

In another aspect, the invention provides a method of enhancing or inducing an anti-tumor immune response in a subject, the method comprising administering to the subject a therapeutically effective amount of a Wnt pathway inhibitor, such as a FZD antagonist.

In certain embodiments of each of the aforementioned aspects, as well as other aspects and embodiments described elsewhere herein, the Wnt pathway inhibitor is a FZD antagonist. In some embodiments, the FZD antagonist is an antibody, for example, an antibody that specifically binds a FZD protein or portion thereof. In some embodiments, the Wnt pathway inhibitor is an antibody that specifically binds at least one human FZD protein. In some embodiments, the Wnt pathway inhibitor is an antibody that specifically binds at least one human FZD protein selected from the group consisting of: FZD1, FZD2, FZD3, FZD4, FZD5, FZD6, FZD7, FZD8, FZD5, and FZD10. In some embodiments, the Wnt pathway inhibitor is an antibody that specifically binds at least one human FZD protein selected from the group consisting of: FZD1, FZD2, FZD5, FZD7, and FZD8. In some embodiments, the Wnt pathway inhibitor is an antibody that comprises a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and/or a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments of each of the aforementioned aspects, as well as other aspects and embodiments described elsewhere herein, the Wnt pathway inhibitor is an antibody that comprises (a) a heavy chain variable region having at least about 90%, at least about 95%, or 100% sequence identity to SEQ ID NO:7; and/or (b) a light chain variable region having at least about 90%, at least about 95%, or 100% sequence identity to SEQ ID NO:8. In some embodiments, the antibody comprises (a) a heavy chain having at least about 90%, at least about 95%, or 100% sequence identity to SEQ ID NO:9 or SEQ ID NO:11; and/or (b) a light chain having at least about 90%, at least about 95%, or 100% sequence identity to SEQ ID NO:10 or SEQ ID NO:12. In some embodiments, the Wnt pathway inhibitor is antibody OMP-18R5 (also known referred to as 18R5 or vantictumab).

In certain embodiments of each of the aforementioned aspects, as well as other aspects and embodiments described elsewhere herein, the Wnt pathway inhibitor is Wnt binding agent, for example, an antibody. The antibody may be an antibody that specifically binds at least one human Wnt protein. For example, the antibody may specifically bind at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b.

In certain embodiments of each of the aforementioned aspects, as well as other aspects and embodiments described elsewhere herein, the Wnt pathway inhibitor is a recombinant antibody. In some embodiments, the antibody is a monoclonal antibody, a chimeric antibody, a humanized antibody, or a human antibody. In some embodiments, the antibody is an antibody fragment comprising an antigen-binding site. In certain embodiments, the antibody or antibody fragment is monovalent, monospecific, bivalent, bispecific, or multispecific. In some embodiments, the antibody is an IgG1 antibody or an IgG2 antibody. In certain embodiments, the antibody is isolated. In other embodiments, the antibody is substantially pure.

In certain embodiments of each of the aforementioned aspects, as well as other aspects and embodiments described elsewhere herein, the Wnt pathway inhibitor is a soluble receptor. In some embodiments, the soluble receptor comprises or consists essentially of the Fri domain of a human FZD protein. In some embodiments, the Fri domain comprises or consists essentially of the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Fri domain comprises or consists essentially of the Fri domain of FZD8. In some embodiments, the Fri domain of the human FZD protein comprises or consists essentially a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and SEQ ID NO:33. In some embodiments, the Fri domain comprises or consists essentially of SEQ ID NO:20. In some embodiments, the Fri domain comprises or consists essentially of SEQ ID NO:21. In some embodiments, the Fri domain comprises or consists essentially of SEQ ID NO:33.

In some embodiments of the methods described herein, the soluble receptor comprises a non-FZD polypeptide. In some embodiments, the non-FZD polypeptide is directly linked to the Fri domain of the human FZD protein. In some embodiments, the non-FZD polypeptide is connected to the Fri domain of the human FZD protein by a linker (e.g., any of those described herein). In some embodiments, the non-FZD polypeptide comprises a human Fc region. In some embodiments, the non-FZD polypeptide comprises or consists essentially of SEQ ID NO:34, SEQ ID NO:35, SEQ ID NO:36, SEQ ID NO:37, or SEQ ID NO:38. In some embodiments, the non-FZD polypeptide comprises or consists essentially of SEQ ID NO:37.

In some embodiments of the methods described herein, the Wnt pathway inhibitor comprises (a) a first polypeptide comprising or consisting essentially of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and (b) a second polypeptide comprising or consisting essentially of SEQ ID NO:34, SEQ ID NO:35, SEQ ID NO:66, SEQ ID NO:37, or SEQ ID NO:38, where the first polypeptide is directly linked to the second polypeptide. In some embodiments, the Wnt pathway inhibitor comprises (a) a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, or SEQ ID NO:23; and (b) a second polypeptide comprising SEQ ID NO:34, SEQ ID NO:35, SEQ ID NO:36, SEQ ID NO:37, or SEQ ID NO:38, where the first polypeptide is connected to the second polypeptide by a linker. In some embodiments, the Wnt pathway inhibitor comprises (a) a first polypeptide comprising SEQ ID NO:20 or SEQ ID NO:21; and (b) a second polypeptide comprising SEQ ID NO:37, where the first polypeptide is directly linked to the second polypeptide. In some embodiments, the Wnt pathway inhibitor comprises (a) a first polypeptide comprising or consisting essentially of SEQ ID NO:20 or SEQ ID NO:21; and (b) a second polypeptide comprising or consisting essentially of SEQ ID NO:37, where the first polypeptide is connected to the second polypeptide by a linker. In some embodiments, the Wnt pathway inhibitor comprises or consists essentially of SEQ ID NO:39, SEQ ID NO:40, or SEQ ID NO:41. In some embodiments, the Wnt pathway inhibitor is FZD8-Fc soluble receptor OMP-54F28 (also known as 54F28 or ipafricept) or has an amino acid sequence at least 85%, 90%, 95%, or 99% identical to the sequence of OMP-54F28. In particular embodiments, the amino acid sequence is at least 95% identical to the sequence of OMP-54F28.

In some embodiments, the Wnt inhibitor is a small molecule, e.g., any of those described herein.

In certain embodiments of each of the aforementioned aspects, as well as other aspects and embodiments described elsewhere herein, the Wnt pathway inhibitor enhances the activity of the immunotherapeutic agent.

In certain embodiments of each of the aforementioned aspects, as well as other aspects and embodiments described elsewhere herein, the immunotherapeutic agent enhances the activity of the Wnt pathway inhibitor.

In certain embodiments of each of the aforementioned aspects, as well as other aspects and embodiments described elsewhere herein, the Wnt pathway inhibitor and the immunotherapeutic agent act synergistically.

In certain embodiments of each of the aforementioned aspects, as well as other aspects and embodiments described elsewhere herein, the immunotherapeutic agent is an agent that modulates immune responses. In some embodiments, the immunotherapeutic agent is an agent that enhances anti-tumor immune responses. In some embodiments, the immunotherapeutic agent is an agent that increases cell-mediated immunity. In some embodiments, the immunotherapeutic agent is an agent that increases T-cell activity. In some embodiments, the immunotherapeutic agent is an agent that increases cytolytic T-cell (CTL) activity. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent that inhibits Treg activity. In some embodiments, the immunotherapeutic agent is an agent that inhibits the activity of inhibitory immune checkpoint receptors.

In some embodiments, the immunotherapeutic agent is a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, or an immunostimulatory oligonucleotide. In some embodiments, the immunotherapeutic agent is an immune checkpoint modulator (e.g., an immune checkpoint inhibitor).

In some embodiments, the immunotherapeutic agent is an agent that inhibits the activity of PD-1. In some embodiments, the immunotherapeutic agent is an agent that inhibits the activity of PD-L1 and/or PD-L2. In some embodiments, the immunotherapeutic agent is an agent that inhibits the activity of CTLA-4. In some embodiments, the immunotherapeutic agent is an agent that inhibits the activity of CD80 and/or CD86. In some embodiments, the immunotherapeutic agent is an agent that inhibits the activity of TIGIT. In some embodiments, the immunotherapeutic agent is an agent that inhibits the activity of KIR. In some embodiments, the immunotherapeutic agent is an agent that enhances or stimulates the activity of activating immune checkpoint receptors.

In some of the embodiments of the methods described herein, the immunotherapeutic agent is a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, or an IDO1 antagonist.

In some embodiments, the PD-1 antagonist is an antibody that specifically binds PD-1. In some embodiments, the antibody that binds PD-1 is pembrolizumab (KEYTRUDA, MK-3475; Merck), pidilizumab (CT-011; Curetech Ltd.), nivolumab (OPDIVO, BMS-936558, MDX-1106; Bristol Myer Squibb), MEDI0680 (AMP-514; AstraZenenca/MedImmune), REGN2810 (Regeneron Pharmaceuticals), BGB-A317 (BeiGene Ltd.), PDR-001 (Novartis), or STI-A1110 (Sorrento Therapeutics). In some embodiments, the antibody that binds PD-1 is described in PCT Publication WO 2014/179664, for example, an antibody identified as APE2058, APE1922, APE1923, APE1924, APE 1950, or APE1963 (Anaptysbio), or an antibody containing the CDR regions of any of these antibodies. In other embodiments, the PD-1 antagonist is a fusion protein that includes the extracellular domain of PD-L1 or PD-L2, for example, AMP-224 (AstraZeneca/MedImmune). In other embodiments, the PD-1 antagonist is a peptide inhibitor, for example, AUNP-12 (Aurigene).

In some embodiments, the PD-L1 antagonist is an antibody that specifically binds PD-L1. In some embodiments, the antibody that binds PD-L1 is atezolizumab (RG7446, MPDL3280A; Genentech), MEDI4736 (AstraZeneca/MedImmune), BMS-936559 (MDX-1105; Bristol Myers Squibb), avelumab (MSB0010718C; Merck KGaA), KD033 (Kadmon), the antibody portion of KD033, or STI-A1014 (Sorrento Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies.

In some embodiments, the CTLA-4 antagonist is an antibody that specifically binds CTLA-4. In some embodiments, the antibody that binds CTLA-4 is ipilimumab (YERVOY; Bristol Myer Squibb) or tremelimumab (CP-675,206; Pfizer). In some embodiments, the CTLA-4 antagonist a CTLA-4 fusion protein or soluble CTLA-4 receptor, for example, KARR-102 (Kahr Medical Ltd.).

In some embodiments, the LAG3 antagonist is an antibody that specifically binds LAG3. In some embodiments, the antibody that binds LAG3 is IMP701 (Prima BioMed), IMP731 (Prima BioMed/GlaxoSmithKline), BMS-986016 (Bristol Myer Squibb), LAG525 (Novartis), and GSK2831781 (GlaxoSmithKline). In some embodiments, the LAG3 antagonist includes a soluble LAG3 receptor, for example, IMP321 (Prima BioMed).

In some embodiments, the KIR antagonist is an antibody that specifically binds KIR. In some embodiments, the antibody that binds KIR is lirilumab (Bristol Myer Squibb/Innate Pharma).

In some embodiments, the immune checkpoint enhancer or stimulator is a CD28 agonist, a 4-1BB agonist, an OX40 agonist, a CD27 agonist, a CD80 agonist, a CD86 agonist, a CD40 agonist, or a GITR agonist.

In some embodiments, the OX40 agonist includes OX40 ligand, or an OX40-binding portion thereof. For example, the OX40 agonist may be MEDI6383 (AstraZeneca). In some embodiments, the OX40 agonist is an antibody that specifically binds OX40. In some embodiments, the antibody that binds OX40 is MEDI6469 (AstraZeneca/MedImmune), MEDI0562 (AstraZeneca/MedImmune), or MOXR0916 (RG7888; Genentech). In some embodiments, the OX40 agonist is a vector (e.g., an expression vector or virus, such as an adenovirus) capable of expressing OX40 ligand. In some embodiments the OX40-expressing vector is Delta-24-RGDOX (DNAtrix) or DNX2401 (DNAtrix).

In some embodiments, the 4-1BB (CD137) agonist is a binding molecule, such as an anticalin. In some embodiments, the anticalin is PRS-343 (Pieris AG). In some embodiments, the 4-1BB agonist is an antibody that specifically binds 4-1BB. In some embodiments, antibody that binds 4-1BB is PF-2566 (PF-05082566; Pfizer) or urelumab (BMS-663513; Bristol Myer Squibb).

In some embodiments, the CD27 agonist is an antibody that specifically binds CD27. In some embodiments, the antibody that binds CD27 is varlilumab (CDX-1127; Celldex).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518 (GITR, Inc.), MK-4166 (Merck), or INBRX-110 (Five Prime Therapeutics/Inhibrx).

In some embodiments, where the Wnt pathway inhibitor and immunotherapeutic agent together are a bispecific antibody. For example, the bispecific antibody may specifically bind a member of the Wnt pathway (e.g., a FZD protein or Wnt) and immune checkpoint (e.g., any described herein, such as PD-1, PD-L1, PD-L2, or CTLA-4, LAG-3, OX40, or CD27). In particular embodiments, the bispecific antibody specifically binds a human FZD protein and one of PD-1, PD-L1, and CTLA-4.

In some embodiments, the immunotherapeutic agent is a cytokine, for example, a chemokine, an interferon, an interleukin, lymphokine, or a member of the tumor necrosis factor family. In some embodiments, the cytokine is IL-2, IL15, or interferon-gamma.

In some embodiments of any of the above aspects or those described elsewhere herein, the cancer is selected from the group consisting of lung cancer, pancreatic cancer, breast cancer, colon cancer, colorectal cancer, melanoma, gastrointestinal cancer, gastric cancer, renal cancer, ovarian cancer, liver cancer, endometrial cancer, kidney cancer, prostate cancer, thyroid cancer, neuroblastoma, glioma, glioblastoma, glioblastoma multiforme, cervical cancer, stomach cancer, bladder cancer, head and neck cancer, and hepatoma.

In some embodiments of any of the above aspects or those described elsewhere herein, the tumor is selected from the group consisting of lung tumor, pancreatic tumor, breast tumor, colon tumor, colorectal tumor, melanoma, gastrointestinal tumor, gastric tumor, renal tumor, ovarian tumor, liver tumor, endometrial tumor, kidney tumor, prostate tumor, thyroid tumor, neuroblastoma, glioma, glioblastoma, glioblastoma multiforme, cervical tumor, stomach tumor, bladder tumor, head and neck tumor, and hepatoma.

In some embodiments of any of the above aspects or those described elsewhere herein, the subject's cancer or tumor does not respond to immune checkpoint inhibition (e.g., to any immune checkpoint inhibitor described herein, such as a PD-1 antagonist or PD-L1 antagonist) or the subject's cancer or tumor has progressed following an initial response to immune checkpoint inhibition (e.g., to any immune checkpoint inhibitor described herein, such as a PD-1 antagonist or PD-L1 antagonist).

In some embodiments of any of the above aspects or those described elsewhere herein, the subject is a human.

### BRIEF DESCRIPTION OF THE DRAWINGS

FIG. 1 shows the effect of vantictumab (18R5) and ipafricept (54F28) alone and in combination with an anti-CTLA4 antibody on tumor cell growth.

FIGS. 2A and 2B show the effect of Wnt inhibition on T cell induction (FIG. 2A) and T cell cytotoxicity (FIG. 2B).

FIGS. 3A-3D show the effect of anti-CTLA-4 and anti-PD-L1 antibody treatment on tumor growth in the presence or absence of vantictumab (18R5). FIG. 3A shows average results. FIGS. 3B-3D show tumor measure growth measurements from individual control animals (FIG. 3B), animals receiving a control antibody along with anti-CTLA-4 and anti-PD-L1 antibodies (FIG. 3C), and animals receiving vantictumab along with anti-CTLA-4 and anti-PD-L1 antibodies (FIG. 3D).

FIGS. 4A and 4B show changes in interferon-gamma (FIG. 4A) and IL-2 (FIG. 4B) in splenocytes following treatment with control antibodies, anti-CTLA-4 and anti-PD-L1 antibodies, or vantictumab along with anti-CTLA-4 and anti-PD-L1 antibodies.

FIG. 5 shows changes in T cell cytotoxicity following treatment with control antibodies, anti-CTLA-4 and anti-PD-L1 antibodies, or vantictumab along with anti-CTLA-4 and anti-PD-L1 antibodies.

FIGS. 6A and 6B show increases in tumor-infiltrating CD4⁺ (FIG. 6A) and CD8⁺ (FIG. 6B) T-cells in cells receiving vantictumab along with anti-CTLA-4 and anti-PD-L1 antibodies, as compared to those receiving anti-CTLA-4 and anti-PD-L1 antibodies without vantictumab or control antibodies.

FIG. 7 shows changes in 4T1 tumor size in mice receiving saline, murinized 18R5 (m18R5), anti-PD1 antibody, docetaxel, anti-PD1+docetaxel, m18R5+anti-PD1, m18R5+docetaxel, or 18R5+anti-PD1+docetaxel. The lower eight graphs show the results from each individual animal treated.

FIGS. 8A-8C show changes in tumor dendritic cell frequency (CD103 and CD8a CD3e⁻; FIGS. 8A and 8B), and splenic dendritic cells CD8a⁺CD3e⁻; FIG. 8C) in 4T1 tumor-implanted mice receiving saline, m18R5, anti-PD1 antibody, docetaxel, anti-PD1+docetaxel, m18R5+anti-PD1, m18R5+docetaxel, or 18R5+anti-PD1+docetaxel.

FIGS. 9A-9C show changes in the frequency of tumor T cells (CD8a⁺CD3e⁺ and CD3⁺; FIGS. 9A and 9B) and splenic T regulatory cells (FIG. 9C) in 4T1 tumor-implanted mice receiving saline, m18R5, anti-PD1 antibody, docetaxel, anti-PD1+docetaxel, m18R5+anti-PD1, m18R5+docetaxel, or 18R5+anti-PD1+docetaxel.

FIGS. 10A-10D show changes in IL17a (FIGS. 10A and 10B) and IL2 (FIG. 10C and FIG. 10D) secreted by splenocytes from 4T1 tumor-implanted mice.

FIG. 11 shows changes in MC38 tumor size in mice receiving saline, 54F28, anti-PD1 antibody, or 54F28+anti-PD1. The lower four graphs show the results from each individual animal treated.

FIG. 12 shows the percentage of tumors in each experimental group that were below 500 mm³ in volume following treatment in MC38-implated mice.

FIGS. 13A-13B show changes in IL2 (FIG. 13A) and IL17a (FIG. 13B) secreted by splenocytes from MC38-implanted mice.

### DETAILED DESCRIPTION OF THE INVENTION

The present invention provides methods of modulating immune responses, particularly anti-tumor immune responses, methods of inhibiting tumor growth, and methods of treating cancer. The methods provided herein comprise administering to a subject a therapeutically effective amount of a Wnt pathway inhibitor in combination with a therapeutically effective amount of an immunotherapeutic agent. In some embodiments, the Wnt pathway inhibitor is a FZD antagonist, such as an anti-FZD antibody or soluble FZD receptor. In some embodiments, the Wnt pathway inhibitor is an antibody that specifically binds at least one human FZD protein. In some embodiments, the Wnt pathway inhibitor is a soluble receptor comprising the Fri domain of a FZD protein, for example, human FZD8. In some embodiments, the immunotherapeutic agent includes but is not limited to, a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

### I. Definitions

To facilitate an understanding of the present invention, a number of terms and phrases are defined below.

The terms “antagonist” and “antagonistic” as used herein refer to any molecule that partially or fully blocks, inhibits, reduces, or neutralizes a biological activity of a target and/or signaling pathway. The term “antagonist” is used herein to include any molecule that partially or fully blocks, inhibits, reduces, or neutralizes the activity of a protein. Suitable antagonist molecules include, but are not limited to, antagonist antibodies, antibody fragments, soluble receptors, and small molecules.

The terms “agonist” and “agonistic” as used herein refer to or describe an agent that is capable of, directly or indirectly, substantially inducing, activating, promoting, increasing, or enhancing the biological activity of a target and/or a signaling pathway. The term “agonist” is used herein to include any agent that partially or fully induces, activates, promotes, increases, or enhances the activity of a protein. Suitable agonists specifically include, but are not limited to, agonist antibodies or fragments thereof, soluble receptors, other fusion proteins, and small molecules.

The term “biomarker” as used herein may include but is not limited to, nucleic acids and proteins, and variants and fragments thereof. A biomarker may include DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest. Biomarker proteins are considered to comprise the entire or partial amino acid sequence of any of the biomarker proteins or polypeptides.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen-binding site within the variable region of the immunoglobulin molecule. As used herein, the term encompasses intact polyclonal antibodies, intact monoclonal antibodies, antibody fragments comprising an antigen-binding site (such as Fab, Fab′, F(ab′)2, and Fv fragments), single chain Fv (scFv) antibodies, multispecific antibodies such as bispecific antibodies, monospecific antibodies, monovalent antibodies, chimeric antibodies, humanized antibodies, human antibodies, fusion proteins comprising an antigen-binding site of an antibody, and any other modified immunoglobulin molecule comprising an antigen-binding site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-characterized subunit structures and three-dimensional configurations. Antibodies can be naked or conjugated to other molecules, including but not limited to, toxins and radioisotopes.

The term “antibody fragment” as used herein refers to a portion of an intact antibody and generally includes the antigenic determining variable region or antigen-binding site of an intact antibody. Examples of antibody fragments include, but are not limited to, Fab, Fab′, F(ab′)2, and Fv fragments, linear antibodies, single chain antibodies, and multispecific antibodies formed from antibody fragments. “Antibody fragment” as used herein comprises at least one antigen-binding site or epitope-binding site.

The term “variable region” of an antibody as used herein refers to the variable region of the antibody light chain, or the variable region of the antibody heavy chain, either alone or in combination. The variable region of the heavy or light chain generally consists of four framework regions connected by three complementarity determining regions (CDRs), also known as “hypervariable regions”. The CDRs in each chain are held together in close proximity by the framework regions and, with the CDRs from the other chain, contribute to the formation of the antigen-binding site of the antibody. There are at least two techniques for determining CDRs: (1) an approach based on cross-species sequence variability (i.e., Kabat et al., 1991, Sequences of Proteins of Immunological Interest, 5th Edition, National Institutes of Health, Bethesda Md.), and (2) an approach based on crystallographic studies of antigen-antibody complexes (Al-Lazikani et al., 1997, J. Mol. Biol., 273:927-948). Combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length antibodies as well as antibody fragments (e.g., Fab, Fab′, F(ab′)2, Fv), single chain (scFv) antibodies, fusion proteins comprising an antibody portion, and any other modified immunoglobulin molecule comprising at least one antigen-binding site. Furthermore, “monoclonal antibody” refers to such antibodies made by any number of techniques, including but not limited to, hybridoma production, phage selection, recombinant expression, and transgenic animals.

The term “humanized antibody” as used herein refers to antibodies that are specific immunoglobulin chains, chimeric immunoglobulins, or fragments thereof that contain minimal non-human sequences. Typically, humanized antibodies are human immunoglobulins in which amino acid residues of the CDRs are replaced by amino acid residues from the CDRs of a non-human species (e.g., mouse, rat, rabbit, or hamster) that have the desired specificity, affinity, and/or binding capability.

The term “human antibody” as used herein refers to an antibody produced by a human or an antibody having an amino acid sequence corresponding to an antibody produced by a human made using any of the techniques known in the art.

The term “chimeric antibody” as used herein refers to an antibody where the amino acid sequence of the immunoglobulin molecule is derived from two or more species. Typically, the variable region of both light and heavy chains corresponds to the variable region of antibodies derived from one species of mammals (e.g., mouse, rat, rabbit, etc.) with the desired specificity, affinity, and/or binding capability, while the constant regions are homologous to the sequences in antibodies derived from another species (usually human).

The term “affinity-matured antibody” as used herein refers to an antibody with one or more alterations in one or more CDRs that result in an improvement in the affinity of the antibody for antigen, compared to a parent antibody that does not possess those alterations(s). In some instances, alterations are made in the framework regions. Preferred affinity-matured antibodies will have nanomolar or even picomolar affinities for the target antigen. Affinity-matured antibodies are produced by procedures known in the art including heavy chain and light chain variable region shuffling, random mutagenesis of CDR and/or framework residues, or site-directed mutagenesis of CDR and/or framework residues.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and non-contiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5, or 8-10 amino acids in a unique spatial conformation.

The terms “selectively binds” or “specifically binds” as used herein mean that a binding agent or an antibody reacts or associates more frequently, more rapidly, with greater duration, with greater affinity, or with some combination of the above to the epitope, protein, or target molecule than with alternative substances, including unrelated or related proteins. In certain embodiments “specifically binds” means, for instance, that an antibody binds a target with a K_{D} of about 0.1 mM or less, but more usually less than about In certain embodiments, “specifically binds” means that an antibody binds a target with a K_{D} of at least about 0.1 μM or less, at least about 0.01 μM or less, or at least about 1 nM or less. Because of the sequence identity between homologous proteins in different species, specific binding can include an antibody that recognizes a protein in more than one species. Likewise, because of homology within certain regions of polypeptide sequences of different proteins, specific binding can include an antibody (or other polypeptide or binding agent) that recognizes more than one protein. It is understood that, in certain embodiments, an antibody or binding agent that specifically binds a first target may or may not specifically bind a second target. As such, “specific binding” does not necessarily require (although it can include) exclusive binding, i.e. binding to a single target. Thus, an antibody may, in certain embodiments, specifically bind more than one target. In certain embodiments, multiple targets may be bound by the same antigen-binding site on the antibody. For example, an antibody may, in certain instances, comprise two identical antigen-binding sites, each of which specifically binds the same epitope on two or more proteins. In certain alternative embodiments, an antibody may be bispecific and comprise at least two antigen-binding sites with differing specificities. Generally, but not necessarily, reference to binding means specific binding.

The term “soluble receptor” as used herein refers to an extracellular fragment (or a portion thereof) of a receptor protein preceding the first transmembrane domain of the receptor that can be secreted from a cell in soluble form.

The term “FZD soluble receptor” as used herein refers to an extracellular fragment of a FZD receptor protein preceding the first transmembrane domain of the receptor that can be secreted from a cell in soluble form. FZD soluble receptors comprising the entire extracellular domain (ECD) as well as smaller fragments of the ECD are encompassed by the term. Thus, FZD soluble receptors comprising the Fri domain are also included in this term.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention may be based upon antibodies, in certain embodiments, the polypeptides can occur as single chains or associated chains

The term “amino acid” as used herein refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, gamma-carboxyglutamate, and O-phosphoserine. The phrase “amino acid analog” refers to compounds that have the same basic chemical structure as a naturally occurring amino acid, e.g., an alpha carbon that is bound to an hydrogen, a carboxyl group, an amino group, and an R group, e.g., homoserine, norleucine, methionine sulfoxide, methionine methyl sulfonium. Such analogs can have modified R groups (e.g., norleucine) or modified peptide backbones, but retain the same basic chemical structure as a naturally occurring amino acid. The phrase “amino acid mimetic” refers to chemical compounds that have a structure that is different from the general chemical structure of an amino acid, but that function similarly to a naturally occurring amino acid.

The terms “polynucleotide” and “nucleic acid” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include, but are not limited to, BLAST and BLAST variations, ALIGN and ALIGN variations, Megalign, BestFit, GCG Wisconsin Package, etc. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60, at least about 60-80 nucleotides or amino acid residues in length or any integral value therebetween. In some embodiments, identity exists over a longer region than 60-80 nucleotides or amino acid residues, such as at least about 80-100 nucleotides or amino acid residues, and in some embodiments the sequences are substantially identical over the full length of the sequences being compared, such as the coding region of a nucleotide sequence.

The term “conservative amino acid substitution” as used herein refers to a substitution in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), non-polar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence to the antigen(s). Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

As used herein, a polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cells, or compositions include those which have been purified to a degree that they are no longer in a form in which they are found in nature. In some embodiments, a polypeptide, antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells is characterized by unregulated cell growth. Examples of cancer include, but are not limited to, carcinoma, blastoma, sarcoma, and hematologic cancers such as lymphoma and leukemia.

The terms “proliferative disorder” and “proliferative disease” as used herein refer to disorders associated with abnormal cell proliferation such as cancer.

The terms “tumor” and “neoplasm” as used herein refer to any mass of tissue that results from excessive cell growth or proliferation, either benign (non-cancerous) or malignant (cancerous), including pre-cancerous lesions.

The term “metastasis” as used herein refers to the process by which a cancer spreads or transfers from the site of origin to other regions of the body with the development of a similar cancerous lesion at the new location. A “metastatic” or “metastasizing” cell is generally one that loses adhesive contacts with neighboring cells and migrates from the primary site of disease to invade neighboring tissue sites.

The terms “cancer stem cell” and “CSC” and “tumor stem cell” and “tumor initiating cell” are used interchangeably herein and refer to cells from a cancer or tumor that: (1) have extensive proliferative capacity; 2) are capable of asymmetric cell division to generate one or more types of differentiated cell progeny where the differentiated cells have reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” as used herein refer to the total population of cells derived from a cancer or tumor or pre-cancerous lesion, including both non-tumorigenic cells, which comprise the bulk of the cancer cell population, and tumorigenic cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “subject” as used herein refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to an agent, compound, molecule, etc. approved or approvable by a regulatory agency of the Federal government, a state government, and/or listed in the U.S. Pharmacopeia or other generally recognized pharmacopeia for use in animals, including humans.

The phrases “pharmaceutically acceptable excipient, carrier or adjuvant” and “acceptable pharmaceutical carrier” refer to an excipient, carrier, or adjuvant that can be administered to a subject, together with a therapeutic agent, and which does not destroy the pharmacological activity thereof and is nontoxic when administered in doses sufficient to deliver a therapeutic effect. In general, those of skill in the art and the FDA consider a pharmaceutically acceptable excipient, carrier, or adjuvant to be an inactive ingredient of any formulation or pharmaceutical composition.

The terms “effective amount” and “therapeutically effective amount” and “therapeutic effect” as used herein refer to an amount of a binding agent, an antibody, a polypeptide, a polynucleotide, a small molecule, or other therapeutic agent effective to “treat” a disease or disorder in a subject or mammal. In the case of cancer, the therapeutically effective amount of an agent (e.g., an antibody) has a therapeutic effect and as such can reduce the number of cancer cells; decrease tumorigenicity, tumorigenic frequency, or tumorigenic capacity; reduce the number or frequency of cancer stem cells; reduce tumor size; reduce the cancer cell population; inhibit and/or stop cancer cell infiltration into peripheral organs including, for example, the spread of cancer into soft tissue and bone; inhibit and stop tumor or cancer cell metastasis; inhibit and/or stop tumor or cancer cell growth; relieve to some extent one or more of the symptoms associated with the cancer; reduce morbidity and mortality; improve quality of life; or a combination of such effects. To the extent the agent prevents growth and/or kills existing cancer cells, it can be referred to as cytostatic and/or cytotoxic.

The terms “treating” and “treatment” and “to treat” and “alleviating” and “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus, those in need of treatment include those who already have a disorder; those prone to have a disorder; and those in whom a disorder is to be prevented. In some embodiments, a subject is successfully “treated” according to the methods of the present invention if the patient shows one or more of the following: a reduction in the number of or complete absence of cancer cells; a reduction in tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; inhibition or an absence of tumor growth; relief of one or more symptoms associated with the specific cancer; reduced morbidity and mortality; improvement in quality of life; reduction in tumorigenicity; reduction in the number or frequency of cancer stem cells; or some combination of effects.

The term “biomarker” as used herein may include but is not limited to, nucleic acids and proteins, and variants and fragments thereof. A biomarker may include DNA comprising the entire or partial nucleic acid sequence encoding the biomarker, or the complement of such a sequence. Biomarker nucleic acids useful in the invention are considered to include both DNA and RNA comprising the entire or partial sequence of any of the nucleic acid sequences of interest. Biomarker proteins are considered to comprise the entire or partial amino acid sequence of any of the biomarker proteins or polypeptides.

As used in the present disclosure and claims, the singular forms “a”, “an” and “the” include plural forms unless the context clearly dictates otherwise.

It is understood that wherever embodiments are described herein with the language “comprising” otherwise analogous embodiments described in terms of “consisting of” and/or “consisting essentially of” are also provided. It is also understood that wherever embodiments are described herein with the language “consisting essentially of” otherwise analogous embodiments described in terms of “consisting of” are also provided.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. Methods of Use and Pharmaceutical Compositions

A Wnt pathway inhibitor described herein in combination with an immunotherapeutic agent is useful in a variety of applications including, but not limited to, therapeutic treatment methods, such as immunotherapy for cancer. In certain embodiments, the combination of a Wnt pathway inhibitor (e.g., a FZD receptor antagonist, such as an anti-FZD antibody or a soluble FZD receptor) with an immunotherapeutic agent, is useful for activating, promoting, increasing, and/or enhancing an immune response, inhibiting tumor growth, reducing tumor volume, increasing tumor cell apoptosis, and/or reducing the tumorigenicity of a tumor. The methods of use may be in vitro, ex vivo, or in vivo methods. In some embodiments, the combination of a Wnt pathway inhibitor (e.g., a FZD receptor antagonist, such as an anti-FZD antibody or a soluble FZD receptor) with an immunotherapeutic agent acts as an agonist of an immune response. In some embodiments, the combination of a Wnt pathway inhibitor (e.g., a FZD receptor antagonist, such as an anti-FZD antibody or a soluble FZD receptor) with an immunotherapeutic agent acts as an enhancer, activator, or stimulator of an immune response. In some embodiments, the combination of a Wnt pathway inhibitor (e.g., a FZD receptor antagonist, such as an anti-FZD antibody or a soluble FZD receptor) with an immunotherapeutic agent acts as an agonist of an anti-tumor immune response. In some embodiments, the combination of a Wnt pathway inhibitor (e.g., a FZD receptor antagonist, such as an anti-FZD antibody or a soluble FZD receptor) with an immunotherapeutic agent works as an antagonist of the PD-1 pathway. In some embodiments, the combination of a Wnt pathway inhibitor (e.g., a FZD receptor antagonist, such as an anti-FZD antibody or a soluble FZD receptor) with an immunotherapeutic agent works as an antagonist of PD-1 or PD-1 activity. In some embodiments, the combination of a Wnt pathway inhibitor (e.g., a FZD receptor antagonist, such as an anti-FZD antibody or a soluble FZD receptor) with an immunotherapeutic agent works as an antagonist of PD-L1 or PD-L1 activity. In some embodiments, the combination of a Wnt pathway inhibitor (e.g., a FZD receptor antagonist, such as an anti-FZD antibody or a soluble FZD receptor) with an immunotherapeutic agent works as an antagonist of the CTLA-4 pathway. In some embodiments, the combination of a Wnt pathway inhibitor (e.g., a FZD receptor antagonist, such as an anti-FZD antibody or a soluble FZD receptor) with an immunotherapeutic agent works as an antagonist of CTLA-4 or CTLA-4 activity. In some embodiments, the combination of a Wnt pathway inhibitor (e.g., a FZD receptor antagonist, such as an anti-FZD antibody or a soluble FZD receptor) with an immunotherapeutic agent works as an antagonist of Tim-3 or Tim-3 activity. In some embodiments, the combination of a Wnt pathway inhibitor (e.g., a FZD receptor antagonist, such as an anti-FZD antibody or a soluble FZD receptor) with an immunotherapeutic agent works as an antagonist of LAG3 or LAG3 activity. In some embodiments, the combination of a Wnt pathway inhibitor (e.g., a FZD receptor antagonist, such as an anti-FZD antibody or a soluble FZD receptor) with an immunotherapeutic agent works as an antagonist of TIGIT or TIGIT activity. In some embodiments, the combination of a Wnt pathway inhibitor (e.g., a FZD receptor antagonist, such as an anti-FZD antibody or a soluble FZD receptor) with an immunotherapeutic agent works as an antagonist of KIR or KIR activity.

In certain embodiments of the methods described herein, a method of inhibiting tumor growth comprises contacting tumor cells with an effective amount of a Wnt pathway inhibitor in combination with an effective amount of an immunotherapeutic agent. The method may be in vivo or in vitro. In certain embodiments, the tumor is in a subject, and contacting tumor cells with the Wnt pathway inhibitor and the immunotherapeutic agent comprises administering a therapeutically effective amount of each of the agents to the subject. In some embodiments, a method of inhibiting tumor growth comprises administering to a subject a therapeutically effective amount of a Wnt pathway inhibitor and a therapeutically effective amount of an immunotherapeutic agent, where the Wnt pathway inhibitor is a FZD receptor antagonist, such as an anti-FZD antibody or a soluble FZD receptor. In some embodiments, the Wnt pathway inhibitor and the immunotherapeutic agent inhibit tumor growth by inhibiting or suppressing regulatory T-cell (Treg) activity. In some embodiments, the Wnt pathway inhibitor and the immunotherapeutic agent inhibit tumor growth by increasing cytolytic cell activity. In some embodiments, the Wnt pathway inhibitor and the immunotherapeutic agent inhibit tumor growth by increasing NK cell activity. In some embodiments, the Wnt pathway inhibitor and the immunotherapeutic agent inhibit tumor growth by increasing cytolytic T-cell activity. In some embodiments, the Wnt pathway inhibitor and the immunotherapeutic agent inhibit tumor growth by increasing CD8+ cytolytic T-cell activity. In some embodiments, the Wnt pathway inhibitor and the immunotherapeutic agent inhibit tumor growth by decreasing PD-1 expression on T-cells. In some embodiments, the Wnt pathway inhibitor and the immunotherapeutic agent inhibit tumor growth by decreasing the number or percentage of PD-1 expressing T-cells. In some embodiments, the Wnt pathway inhibitor and the immunotherapeutic agent inhibit tumor growth by decreasing the number or percentage of myeloid-derived suppressor cells (M-MDSCs). In some embodiments, the Wnt pathway inhibitor and the immunotherapeutic agent inhibit tumor growth by increasing the number or percentage of activated myeloid cells. In some embodiments, the Wnt pathway inhibitor and the immunotherapeutic agent inhibit tumor growth by increasing the number or percentage of memory T-cells. In some embodiments, the Wnt pathway inhibitor and the immunotherapeutic agent inhibit tumor growth by increasing IFN-gamma production. In some embodiments, the Wnt pathway inhibitor and the immunotherapeutic agent inhibit tumor growth by increasing IL-2 production. In some embodiments, the Wnt pathway inhibitor and the immunotherapeutic agent inhibit tumor growth by decreasing IL-17 production. In some embodiments, the Wnt pathway inhibitor and the immunotherapeutic agent inhibit tumor growth by decreasing IL-6 production.

In certain embodiments of the methods described herein, a method of treating cancer comprises administering to a subject a therapeutically effective amount of a Wnt pathway inhibitor in combination with a therapeutically effective amount of an immunotherapeutic agent. In some embodiments, a method of treating cancer comprises administering to a subject a therapeutically effective amount of a Wnt pathway inhibitor and a therapeutically effective amount of an immunotherapeutic agent, where the Wnt pathway inhibitor is a FZD antagonist. In some embodiments, the Wnt pathway inhibitor and the immunotherapeutic agent treat cancer by inhibiting or suppressing Treg activity. In some embodiments, the Wnt pathway inhibitor and the immunotherapeutic agent treat cancer by increasing cytolytic cell activity. In some embodiments, the Wnt pathway inhibitor and the immunotherapeutic agent treat cancer by increasing NK cell activity. In some embodiments, the Wnt pathway inhibitor and the immunotherapeutic agent treat cancer by increasing cytolytic T-cell activity. In some embodiments, the Wnt pathway inhibitor and the immunotherapeutic agent treat cancer by increasing CD8+ cytolytic T-cell activity. In some embodiments, the Wnt pathway inhibitor and the immunotherapeutic agent treat cancer by decreasing PD-1 expression on T-cells. In some embodiments, the Wnt pathway inhibitor and the immunotherapeutic agent treat cancer by decreasing the number or percentage of PD-1 expressing T-cells. In some embodiments, the Wnt pathway inhibitor and the immunotherapeutic agent treat cancer by decreasing the number or percentage of M-MDSCs. In some embodiments, the Wnt pathway inhibitor and the immunotherapeutic agent treat cancer by increasing the number or percentage of activated myeloid cells. In some embodiments, the Wnt pathway inhibitor and the immunotherapeutic agent treat cancer by increasing the number or percentage of memory T-cells. In some embodiments, the Wnt pathway inhibitor and the immunotherapeutic agent treat cancer by increasing IFN-gamma production. In some embodiments, the Wnt pathway inhibitor and the immunotherapeutic agent treat cancer by increasing IL-2 production. In some embodiments, the Wnt pathway inhibitor and the immunotherapeutic agent treat cancer by decreasing IL-17 production. In some embodiments, the Wnt pathway inhibitor and the immunotherapeutic agent treat cancer by decreasing IL-6 production.

In certain embodiments of the methods described herein, a method of cancer immunotherapy comprises administering to a subject a therapeutically effective amount of a Wnt pathway inhibitor in combination with a therapeutically effective amount of an immunotherapeutic agent, and where the combination results in enhanced therapeutic efficacy as compared to administration of either agent alone. In some embodiments, the Wnt pathway inhibitor is a FZD receptor antagonist, such as an anti-FZD antibody or a soluble FZD receptor.

In certain embodiments of the methods described herein, a method of inhibiting the activity of Tregs comprises administering to a subject a therapeutically effective amount of a Wnt pathway inhibitor in combination with a therapeutically effective amount of an immunotherapeutic agent. In some embodiments, a method of inhibiting the activity of Tregs comprises administering to a subject a therapeutically effective amount of a Wnt pathway inhibitor and a therapeutically effective amount of an immunotherapeutic agent, where the Wnt pathway inhibitor is a FZD receptor antagonist, such as an anti-FZD antibody or a soluble a FZD receptor.

In certain embodiments of the methods described herein, a method of inhibiting the suppression of immune responses by Tregs comprises administering to a subject a therapeutically effective amount of a Wnt pathway inhibitor in combination with a therapeutically effective amount of an immunotherapeutic agent. In some embodiments, a method of inhibiting the suppression of immune responses by Tregs comprises administering to a subject a therapeutically effective amount of a Wnt pathway inhibitor and a therapeutically effective amount of an immunotherapeutic agent, where the Wnt pathway inhibitor is a FZD receptor antagonist, such as an anti-FZD antibody or a soluble FZD receptor.

In certain embodiments of the methods described herein, a method of increasing T cell infiltration into a tumor comprises administering to a subject a therapeutically effective amount of a Wnt pathway inhibitor in combination with a therapeutically effective amount of an immunotherapeutic agent. In some embodiments, a method of increasing T cell infiltration into a tumor comprises administering to a subject a therapeutically effective amount of a Wnt pathway inhibitor and a therapeutically effective amount of an immunotherapeutic agent, where the Wnt pathway inhibitor is a FZD receptor antagonist, such as an anti-FZD antibody or a soluble FZD receptor.

In certain embodiments of the methods described herein, a method of increasing T cell cytotoxicity to a tumor comprises administering to a subject a therapeutically effective amount of a Wnt pathway inhibitor in combination with a therapeutically effective amount of an immunotherapeutic agent. In some embodiments, a method of increasing T cell cytotoxicity to a tumor comprises administering to a subject a therapeutically effective amount of a Wnt pathway inhibitor and a therapeutically effective amount of an immunotherapeutic agent, where the Wnt pathway inhibitor is a FZD receptor antagonist, such as an anti-FZD antibody or a soluble FZD receptor.

In certain embodiments of the methods described herein, a method of increasing tumor cell lysis comprises administering to a subject a therapeutically effective amount of a Wnt pathway inhibitor and an immunotherapeutic agent. In some embodiments, a method of increasing tumor cell lysis comprises administering to a subject a therapeutically effective amount of a Wnt pathway inhibitor and a therapeutically effective amount of an immunotherapeutic agent, where the Wnt pathway inhibitor is a FZD receptor antagonist, such as an anti-FZD antibody or a soluble FZD receptor.

In certain embodiments of the methods described herein, a method of inhibiting tumor relapse or tumor regrowth comprises administering to a subject a therapeutically effective amount of a Wnt pathway inhibitor in combination with a therapeutically effective amount of an immunotherapeutic agent. In some embodiments, a method of inhibiting tumor relapse or tumor regrowth comprises administering to a subject a therapeutically effective amount of a Wnt pathway inhibitor and a therapeutically effective amount of immunotherapeutic agent, where the Wnt pathway inhibitor is a FZD receptor antagonist, such as an anti-FZD antibody or a soluble FZD receptor.

In certain embodiments of the methods described herein, a method to increase the efficacy of an immune checkpoint modulator comprises administering to a subject a therapeutically effective amount of a Wnt pathway inhibitor in combination with a therapeutically effective amount of an immune checkpoint modulator. In some embodiments, a method to increase the efficacy of an immune checkpoint inhibitor comprises administering to a subject a therapeutically effective amount of a Wnt pathway inhibitor and a therapeutically effective amount of an immune checkpoint modulator, where the Wnt pathway inhibitor is a FZD receptor antagonist, such as an anti-FZD antibody or a soluble FZD receptor. In some embodiments, the immune checkpoint modulator is an immune checkpoint inhibitor. In some embodiments, the immune checkpoint modulator is an immune checkpoint enhancer or stimulator.

In certain embodiments of the methods described herein, a method of enhancing treatment for a subject who is being treated with an immune checkpoint modulator comprises administering to the subject a therapeutically effective amount of a FZD receptor antagonist, such as an anti-FZD antibody or a soluble FZD receptor. In some embodiments, the immune checkpoint modulator is an immune checkpoint inhibitor. In some embodiments, the immune checkpoint inhibitor is a PD-1 antagonist. In some embodiments, the immune checkpoint inhibitor is an antibody that specifically binds PD-1. In some embodiments, the immune checkpoint inhibitor is a PD-L1 antagonist. In some embodiments, immune checkpoint inhibitor is an antibody that specifically binds PD-L1. In some embodiments, the immune checkpoint inhibitor is a CTLA-4 antagonist. In some embodiments, the immune checkpoint inhibitor is an antibody that specifically binds CTLA-4.

In some embodiments, the method of inhibiting tumor growth comprises contacting the tumor or tumor cells with a Wnt pathway inhibitor (e.g., a FZD receptor antagonist, such as an anti-FZD antibody or a soluble FZD receptor) and an immunotherapeutic agent in vivo. In certain embodiments, contacting a tumor or tumor cell with a Wnt pathway inhibitor and an immunotherapeutic agent is undertaken in an animal model. For example, a Wnt pathway inhibitor and an immunotherapeutic agent may be administered to mice which have tumors. In some embodiments, a Wnt pathway inhibitor and an immunotherapeutic agent increases, promotes, and/or enhances the activity of immune cells in the mice. In some embodiments, a Wnt pathway inhibitor and an immunotherapeutic agent are administered to an animal to inhibit growth of tumors. In some embodiments, a Wnt pathway inhibitor and an immunotherapeutic agent are administered at the same time or shortly after introduction of tumor cells into the animal (preventative model). In some embodiments, a Wnt pathway inhibitor and an immunotherapeutic agent are administered after the tumor cells have become established and grown to a tumor of specific size (therapeutic model).

In certain embodiments, a method of inhibiting growth of a tumor comprises administering to a subject a therapeutically effective amount of a Wnt pathway inhibitor (e.g., a FZD receptor antagonist, such as an anti-FZD antibody or a soluble FZD receptor) and a therapeutically effective amount of an immunotherapeutic agent. In certain embodiments, the subject is a human. In certain embodiments, the subject has a tumor or has had a tumor which was removed. In certain embodiments, the tumor comprises cancer stem cells. In certain embodiments, the frequency of cancer stem cells in the tumor is reduced by administration of the Wnt pathway inhibitor.

The invention also provides a method of reducing or preventing metastasis in a subject comprising administering to the subject a therapeutically effective amount of a Wnt pathway inhibitor and a therapeutically effective amount of an immunotherapeutic agent. In some embodiments, the Wnt pathway inhibitor is a FZD receptor antagonist, such as an anti-FZD antibody or a soluble FZD receptor. In some embodiments, the reduction or prevention of metastasis comprises inhibiting invasiveness of a tumor. In certain embodiments, the subject is a human. In certain embodiments, the subject has a tumor or has had a tumor removed.

In addition, the invention provides a method of reducing the tumorigenicity of a tumor in a subject, comprising administering to a subject a therapeutically effective amount of a Wnt pathway inhibitor and a therapeutically effective amount of an immunotherapeutic agent. In some embodiments, the Wnt pathway inhibitor is a FZD receptor antagonist, such as an anti-FZD antibody or a soluble FZD receptor. In certain embodiments, the tumor comprises cancer stem cells. In some embodiments, the tumorigenicity of a tumor is reduced by reducing the frequency of cancer stem cells in the tumor. In certain embodiments, the frequency of cancer stem cells in the tumor is reduced by administration of the Wnt pathway inhibitor and the immunotherapeutic agent. In some embodiments, the tumorigenicity of the tumor is reduced by inducing apoptosis of the tumor cells. In some embodiments, the tumorigenicity of the tumor is reduced by increasing apoptosis of the tumor cells.

The invention also provides a method of reducing cancer stem cell frequency in a tumor comprising cancer stem cells, the method comprising administering to a subject a therapeutically effective amount of a Wnt pathway inhibitor and a therapeutically effective amount of an immunotherapeutic agent. In some embodiments, the Wnt pathway inhibitor is a FZD receptor antagonist, such as an anti-FZD antibody or a soluble FZD receptor. In certain embodiments, the Wnt pathway inhibitor in combination with an immunotherapeutic agent is capable of reducing the tumorigenicity of a tumor comprising cancer stem cells in an animal model, such as a mouse model. In certain embodiments, the number or frequency of cancer stem cells in a treated tumor is reduced by at least about two-fold, about three-fold, about five-fold, about ten-fold, about 50-fold, about 100-fold, or about 1000-fold as compared to the number or frequency of cancer stem cells in an untreated tumor. In certain embodiments, the reduction in the number or frequency of cancer stem cells is determined by limiting dilution assay using an animal model.

In some embodiments of the methods described herein the cancer is a cancer selected from the group consisting of lung cancer, pancreatic cancer, breast cancer, colon cancer, colorectal cancer, melanoma, gastrointestinal cancer, gastric cancer, renal cancer, ovarian cancer, liver cancer, endometrial cancer, kidney cancer, prostate cancer, thyroid cancer, neuroblastoma, glioma, glioblastoma, glioblastoma multiforme, cervical cancer, stomach cancer, bladder cancer, head and neck cancer, and hepatoma. In some embodiments, the cancer is lung cancer. In some embodiments, the cancer is ovarian cancer.

In some embodiments of any of the methods described herein, the Wnt pathway inhibitor is a Wnt-binding agent. In some embodiments, the Wnt pathway inhibitor is a FZD-binding agent. In some embodiments, the Wnt pathway inhibitor is an antibody. In some embodiments, the Wnt pathway inhibitor is an anti-Wnt antibody. In some embodiments, the Wnt pathway inhibitor is an anti-FZD antibody. In some embodiments, the Wnt pathway inhibitor is the antibody OMP-18R5. In some embodiments, the Wnt pathway inhibitor is a soluble receptor. In some embodiments, the Wnt pathway inhibitor is a FZD-Fc soluble receptor. In some embodiments, the Wnt pathway inhibitor is a FZD8-Fc soluble receptor. In some embodiments, the Wnt pathway inhibitor is FZD8-Fc soluble receptor OMP-54F28.

In some embodiments of any of the methods described herein, the Wnt pathway inhibitor is an antibody that specifically binds at least one FZD protein or fragment thereof. In some embodiments, the antibody specifically binds at least one human FZD protein selected from the group consisting of: FZD1, FZD2, FZD3, FZD4, FZD5, FZD6, FZD7, FZD8, FZD9, and FZD10. In some embodiments, the antibody specifically binds at least one human FZD protein selected from the group consisting of: FZD1, FZD2, FZD5, FZD7, and FZD8. In some embodiments, the Wnt pathway inhibitor is an antibody that specifically binds at least one human FZD protein comprising: (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a PD-1 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a PD-L1 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a PD-L2 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CTLA-4 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD80 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD86 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a KIR antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a Tim-3 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a LAG3 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a TIGIT antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD20 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD96 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a IDO1 antagonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD28 agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a 4-1BB agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a OX40 agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD27 agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD80 agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD86 agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a CD40 agonist.

In certain embodiments of any of the methods described herein, the Wnt pathway inhibitor is an antibody comprising (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6) and is administered in combination with a GITR agonist.

In certain embodiments of any of the methods described herein (e.g., those described above), the Wnt pathway inhibitor is an antibody comprising a heavy chain variable region comprising SEQ ID NO:7 and a light chain variable region comprising SEQ ID NO:8.

In some embodiments, the antibody is a monoclonal antibody, a recombinant antibody, a chimeric antibody, a humanized antibody, a human antibody, or an antibody fragment comprising an antigen-binding site. In some embodiments, the antibody is a monospecific antibody or a bispecific antibody. In some embodiments, the antibody is an IgG1 antibody or an IgG2 antibody. In some embodiments, the Wnt pathway inhibitor is the antibody OMP-18R5 (vantictumab).

In certain embodiments of any of the methods described herein, the Wnt pathway inhibitor is a soluble receptor. In some embodiments, the soluble receptor comprises a Fri domain of a human FZD protein. In some embodiments, the Fri domain of the human FZD protein comprises the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Fri domain of the human FZD protein comprises the Fri domain of FZD8. In some embodiments, the Fri domain of the human FZD protein comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, and SEQ ID NO:23.

In certain embodiments of any of the methods described herein, the Wnt pathway inhibitor is a FZD-Fc soluble receptor comprising SEQ ID NO:20 or SEQ ID NO:21, administered in combination with a mitotic inhibitor in a staggered dosing manner. In some embodiments, the Wnt pathway inhibitor is a FZD-Fc soluble receptor comprising SEQ ID NO:20. In some embodiments, the Wnt pathway inhibitor is a FZD-Fc soluble receptor comprising SEQ ID NO:21.

In some embodiments, the Wnt pathway inhibitor is an antibody. In some embodiments, the antibody is a monoclonal antibody. In some embodiments, the antibody is a humanized antibody. In some embodiments, the antibody is a recombinant antibody, a chimeric antibody, a human antibody, or an antibody fragment comprising an antigen-binding site. In some embodiments, the antibody is a monospecific antibody. In some embodiments, the antibody is a bispecific antibody. In some embodiments, the antibody is an IgG1 antibody or an IgG2 antibody.

In some embodiments, the Wnt pathway inhibitor is vantictumab (OMP-18R5)

In some embodiments, the Wnt pathway inhibitor is ipafricept (OMP-54F28).

In certain embodiments of any of the methods described herein, the Wnt pathway inhibitor is an antibody that specifically binds at least one human FZD protein and the immunotherapeutic agent is a PD-1 antagonist. In some embodiments, the Wnt pathway inhibitor is an antibody that specifically binds at least one human FZD protein and the immunotherapeutic agent is a PD-L1 antagonist. In some embodiments, the Wnt pathway inhibitor is an antibody that specifically binds at least one human FZD protein and the immunotherapeutic agent is a PD-L2 antagonist. In some embodiments, the Wnt pathway inhibitor is an antibody that specifically binds human at least one human FZD protein and the immunotherapeutic agent is a CTLA-4 antagonist. In some embodiments, the Wnt pathway inhibitor is an antibody that specifically binds at least one human FZD protein and the immunotherapeutic agent is a CD80 antagonist. In some embodiments, the Wnt pathway inhibitor is an antibody that specifically binds at least one human FZD protein and the immunotherapeutic agent is a CD86 antagonist. In some embodiments, the Wnt pathway inhibitor is an antibody that specifically binds at least one human FZD protein and the immunotherapeutic agent is a KIR antagonist. In some embodiments, the Wnt pathway inhibitor is an antibody that specifically binds at least one human FZD protein and the immunotherapeutic agent is a Tim-3 antagonist. In some embodiments, the Wnt pathway inhibitor is an antibody that specifically binds at least one human FZD protein and the immunotherapeutic agent is a LAG3 antagonist. In some embodiments, the Wnt pathway inhibitor is an antibody that specifically binds at least one human FZD protein and the immunotherapeutic agent is a TIGIT antagonist. In some embodiments, the Wnt pathway inhibitor is an antibody that specifically binds at least one human FZD protein and the immunotherapeutic agent is a CD96 antagonist. In some embodiments, the Wnt pathway inhibitor is an antibody that specifically binds at least one human FZD protein and the immunotherapeutic agent is an IDO1 antagonist. In some embodiments, the Wnt pathway inhibitor is an antibody that specifically binds at least one human FZD protein and the immunotherapeutic agent is a CD28 agonist. In some embodiments, the Wnt pathway inhibitor is an antibody that specifically binds at least one human FZD protein and the immunotherapeutic agent is a 4-1BB agonist. In some embodiments, the Wnt pathway inhibitor is an antibody that specifically binds at least one human FZD protein and the immunotherapeutic agent is an OX40 agonist. In some embodiments, the Wnt pathway inhibitor is an antibody that specifically binds at least one human FZD protein and the immunotherapeutic agent is a CD27 agonist. In some embodiments, the Wnt pathway inhibitor is an antibody that specifically binds at least one human FZD protein and the immunotherapeutic agent is a CD80 agonist. In some embodiments, the Wnt pathway inhibitor is an antibody that specifically binds at least one human FZD protein and the immunotherapeutic agent is a CD86 agonist. In some embodiments, the Wnt pathway inhibitor is an antibody that specifically binds at least one human FZD protein and the immunotherapeutic agent is a CD40 agonist. In some embodiments, the Wnt pathway inhibitor is an antibody that specifically binds at least one human FZD protein and the immunotherapeutic agent is a GITR agonist. In some embodiments, the Wnt pathway inhibitor is an antibody that specifically binds at least one human FZD protein and the immunotherapeutic agent is a cytokine. In some embodiments, the Wnt pathway inhibitor is an antibody that specifically binds at least one human FZD protein and the immunotherapeutic agent is an interferon. In some embodiments, the Wnt pathway inhibitor is an antibody that specifically binds at least one human FZD protein and the immunotherapeutic agent is a lymphokine.

In certain embodiments of any of the methods described herein, the Wnt pathway inhibitor is a soluble receptor comprising a Fri domain of a human FZD protein and the immunotherapeutic agent is a PD-1 antagonist. In some embodiments, the Wnt pathway inhibitor is a soluble receptor comprising a Fri domain of a human FZD protein and the immunotherapeutic agent is a PD-L1 antagonist. In some embodiments, the Wnt pathway inhibitor is a soluble receptor comprising a Fri domain of a human FZD protein and the immunotherapeutic agent is a PD-L2 antagonist. In some embodiments, the Wnt pathway inhibitor is a soluble receptor comprising a Fri domain of a human FZD protein and the immunotherapeutic agent is a CTLA-4 antagonist. In some embodiments, the Wnt pathway inhibitor is a soluble receptor comprising a Fri domain of a human FZD protein and the immunotherapeutic agent is a CD80 antagonist. In some embodiments, the Wnt pathway inhibitor is a soluble receptor comprising a Fri domain of a human FZD protein and the immunotherapeutic agent is a CD86 antagonist. In some embodiments, the Wnt pathway inhibitor is a soluble receptor comprising a Fri domain of a human FZD protein and the immunotherapeutic agent is a KIR antagonist. In some embodiments, the Wnt pathway inhibitor is a soluble receptor comprising a Fri domain of a human FZD protein and the immunotherapeutic agent is a Tim-3 antagonist. In some embodiments, the Wnt pathway inhibitor is a soluble receptor comprising a Fri domain of a human FZD protein and the immunotherapeutic agent is a LAG3 antagonist. In some embodiments, the Wnt pathway inhibitor is a soluble receptor comprising a Fri domain of a human FZD protein and the immunotherapeutic agent is a TIGIT antagonist. In some embodiments, the Wnt pathway inhibitor is a soluble receptor comprising a Fri domain of a human FZD protein and the immunotherapeutic agent is a CD96 antagonist. In some embodiments, the Wnt pathway inhibitor is a soluble receptor comprising a Fri domain of a human FZD protein and the immunotherapeutic agent is an IDO1 antagonist. In some embodiments, the Wnt pathway inhibitor is a soluble receptor comprising a Fri domain of a human FZD protein and the immunotherapeutic agent is a CD28 agonist. In some embodiments, the Wnt pathway inhibitor is a soluble receptor comprising a Fri domain of a human FZD protein and the immunotherapeutic agent is a 4-1BB agonist. In some embodiments, the Wnt pathway inhibitor is a soluble receptor comprising a Fri domain of a human FZD protein and the immunotherapeutic agent is an OX40 agonist. In some embodiments, the Wnt pathway inhibitor is a soluble receptor comprising a Fri domain of a human FZD protein and the immunotherapeutic agent is a CD27 agonist. In some embodiments, the Wnt pathway inhibitor is a soluble receptor comprising a Fri domain of a human FZD protein and the immunotherapeutic agent is a CD80 agonist. In some embodiments, the Wnt pathway inhibitor is a soluble receptor comprising a Fri domain of a human FZD protein and the immunotherapeutic agent is a CD86 agonist. In some embodiments, the Wnt pathway inhibitor is a soluble receptor comprising a Fri domain of a human FZD protein and the immunotherapeutic agent is a CD40 agonist. In some embodiments, the Wnt pathway inhibitor is a soluble receptor comprising a Fri domain of a human FZD protein and the immunotherapeutic agent is a GITR agonist. In some embodiments, the Wnt pathway inhibitor is a soluble receptor comprising a Fri domain of a human FZD protein and the immunotherapeutic agent is a cytokine. In some embodiments, the Wnt pathway inhibitor is a soluble receptor comprising a Fri domain of a human FZD protein and the immunotherapeutic agent is an interferon. In some embodiments, the Wnt pathway inhibitor is a soluble receptor comprising a Fri domain of a human FZD protein and the immunotherapeutic agent is a lymphokine. In any of these embodiments, the human FZD protein can be FZD8.

The present invention further provides compositions comprising Wnt pathway inhibitors and compositions comprising immunotherapeutic agents. In some embodiments, a composition comprises a FZD antagonist described herein. In some embodiments, a composition comprises an antibody that specifically binds at least one human FZD protein described herein. In some embodiments, the composition comprises a soluble receptor comprising a Fri domain of a human FZD protein (e.g., human FZD8) described herein. In some embodiments, a composition comprises an immunotherapeutic agent described herein. In some embodiments, a composition is a pharmaceutical composition comprising a Wnt pathway inhibitor and a pharmaceutically acceptable vehicle. In some embodiments, a composition is a pharmaceutical composition comprising an immunotherapeutic agent and a pharmaceutically acceptable vehicle. The pharmaceutical compositions find use in modulating immune responses in human patients, particularly immune responses to tumors. The pharmaceutical compositions find use in inhibiting tumor cell growth and treating cancer in human patients. The pharmaceutical compositions find use in any of the methods described herein. In some embodiments, a Wnt pathway inhibitor described herein finds use in the manufacture of a medicament for the treatment of cancer in combination with at least one immunotherapeutic agent. In some embodiments, a FZD antagonist described herein finds use in the manufacture of a medicament for the treatment of cancer in combination with at least one immunotherapeutic agent.

Formulations and/or pharmaceutical compositions are prepared for storage and use by combining a therapeutic agent of the present invention with a pharmaceutically acceptable carrier, excipient, and/or stabilizer as a sterile lyophilized powder, aqueous solution, etc. (Remington: The Science and Practice of Pharmacy, 22nd Edition, 2012, Pharmaceutical Press, London). Those of skill in the art generally consider pharmaceutically acceptable carriers, excipients, and/or stabilizers to be inactive ingredients of a formulation or pharmaceutical composition.

Suitable carriers, excipients, or stabilizers comprise nontoxic buffers such as phosphate, citrate, and other organic acids; salts such as sodium chloride; antioxidants including ascorbic acid and methionine; preservatives (e.g. octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3-pentanol; and m-cresol); low molecular weight polypeptides (such as less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose, or sorbitol; salt-forming counter-ions such as sodium; metal complexes (e.g. Zn-protein complexes); and/or non-ionic surfactants such as polysorbate (TWEEN) or polyethylene glycol (PEG).

The therapeutic formulation can be in unit dosage form. Such formulations include tablets, pills, capsules, powders, granules, solutions or suspensions in water or non-aqueous media, or suppositories for oral, parenteral, or rectal administration or for administration by inhalation. In solid compositions such as tablets the principal active ingredient is mixed with a pharmaceutical carrier. As described herein, pharmaceutical carriers are considered to be inactive ingredients of a formulation or composition. Conventional tableting ingredients include corn starch, lactose, sucrose, sorbitol, talc, stearic acid, magnesium stearate, dicalcium phosphate or gums, and other diluents (e.g. water) to form a solid pre-formulation composition containing a homogeneous mixture of a compound of the present invention, or a non-toxic pharmaceutically acceptable salt thereof. The solid pre-formulation composition is then subdivided into unit dosage forms of the type described above. The tablets, pills, etc., of the novel composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, including a number of polymeric acids and mixtures of polymeric acids with such materials as shellac, cetyl alcohol and cellulose acetate.

Pharmaceutical formulations may include the Wnt pathway inhibitors and/or the immunotherapeutic agents of the present invention complexed with liposomes. Liposomes can be generated by the reverse phase evaporation with a lipid composition comprising phosphatidylcholine, cholesterol, and PEG-derivatized phosphatidylethanolamine (PEG-PE). Liposomes are extruded through filters of defined pore size to yield liposomes with the desired diameter.

The Wnt pathway inhibitors and/or immunotherapeutic agents can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 22nd Edition, 2012, Pharmaceutical Press, London.

In addition, sustained-release preparations comprising Wnt pathway inhibitors and/or immunotherapeutic agents can be prepared. Suitable examples of sustained-release preparations include semi-permeable matrices of solid hydrophobic polymers containing the agent, which matrices are in the form of shaped articles (e.g., films or microcapsules). Examples of sustained-release matrices include polyesters, hydrogels such as poly(2-hydroxyethyl-methacrylate) or poly(vinylalcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The Wnt pathway inhibitors and immunotherapeutic agents are administered as appropriate pharmaceutical compositions to a human patient according to known methods. The pharmaceutical compositions can be administered in any number of ways for either local or systemic treatment. Suitable methods of administration include, but are not limited to, intravenous (administration as a bolus or by continuous infusion over a period of time), intraarterial, intramuscular (injection or infusion), intratumoral, intraperitoneal, intracerobrospinal, subcutaneous, intra-articular, intrasynovial, intracranial (e.g., intrathecal or intraventricular), or oral. In additional, administration can be topical, (e.g., transdermal patches, ointments, lotions, creams, gels, drops, suppositories, sprays, liquids and powders) or pulmonary (e.g., by inhalation or insufflation of powders or aerosols, including by nebulizer; intratracheal, intranasal, epidermal and transdermal).

For the treatment of a disease, the appropriate dosage(s) of a Wnt pathway inhibitor in combination with an immunotherapeutic agent of the present invention depends on the type of disease to be treated, the severity and course of the disease, the responsiveness of the disease, whether the inhibitors are administered for therapeutic or preventative purposes, previous therapy, the patient's clinical history, and so on, all at the discretion of the treating physician. The Wnt pathway inhibitor can be administered one time or as a series of treatments spread over several days to several months, or until a cure is effected or a diminution of the disease state is achieved (e.g., reduction in tumor size). The immunotherapeutic agent can be administered one time or as a series of treatments spread over several days to several months, or until a cure is effected or a diminution of the disease state is achieved (e.g., reduction in tumor size). Optimal dosing schedules for each agent can be calculated from measurements of drug accumulation in the body of the patient and will vary depending on the relative potency of an individual agent. The administering physician can determine optimum dosages, dosing methodologies, and repetition rates.

In some embodiments, combined administration includes co-administration in a single pharmaceutical formulation. In some embodiments, combined administration includes using separate formulations and consecutive administration in either order but generally within a time period such that all active agents can exert their biological activities simultaneously. In some embodiments, combined administration includes using separate formulations and a staggered dosing regimen. In some embodiments, combined administration includes using separate formulations and administration in a specific order. In some embodiments, combined administration includes using separate formulations and administration of the agents in a specific order and in a staggered dosing regimen.

In certain embodiments, dosage of a Wnt pathway inhibitor is from about 0.01 μg to about 100 mg/kg of body weight, from about 0.1 μg to about 100 mg/kg of body weight, from about 1 μg to about 100 mg/kg of body weight, from about 1 mg to about 100 mg/kg of body weight, about 1 mg to about 80 mg/kg of body weight from about 10 mg to about 100 mg/kg of body weight, from about 10 mg to about 75 mg/kg of body weight, or from about 10 mg to about 50 mg/kg of body weight. In certain embodiments, the dosage of the Wnt pathway inhibitor is from about 0.1 mg to about 20 mg/kg of body weight. In some embodiments, the Wnt pathway inhibitor is administered to the subject at a dosage of about 2 mg/kg to about 15 mg/kg. In some embodiments, the Wnt pathway inhibitor is administered to the subject at a dosage of about 5 mg/kg to about 15 mg/kg. In certain embodiments, the Wnt pathway inhibitor is administered once or more daily, weekly, monthly, or yearly. In certain embodiments, the Wnt pathway inhibitor is administered once every week, once every two weeks, once every three weeks, or once every four weeks.

In certain embodiments, dosage of an immunotherapeutic agent is from about 0.01 μg to about 100 mg/kg of body weight, from about 0.1 μg to about 100 mg/kg of body weight, from about 1 μg to about 100 mg/kg of body weight, from about 1 mg to about 100 mg/kg of body weight, about 1 mg to about 80 mg/kg of body weight from about 10 mg to about 100 mg/kg of body weight, from about 10 mg to about 75 mg/kg of body weight, or from about 10 mg to about 50 mg/kg of body weight. In certain embodiments, the dosage of an immunotherapeutic agent is from about 0.1 mg to about 20 mg/kg of body weight. In some embodiments, an immunotherapeutic agent is administered to the subject at a dosage of about 2 mg/kg to about 15 mg/kg. In some embodiments, the Wnt pathway inhibitor is administered to the subject at a dosage of about 5 mg/kg to about 15 mg/kg. In certain embodiments, an immunotherapeutic agent is administered once or more daily, weekly, monthly, or yearly. In certain embodiments, an immunotherapeutic agent is administered once every week, once every two weeks, once every three weeks, or once every four weeks.

In some embodiments, dosage of an immunotherapeutic agent is determined by what is considered “standard-of-care” for a particular agent by those of skill in the art (e.g., treating physicians).

In some embodiments, an inhibitor may be administered at an initial higher “loading” dose, followed by one or more lower doses. In some embodiments, the frequency of administration may also change. In some embodiments, a dosing regimen may comprise administering an initial dose, followed by additional doses (or “maintenance” doses) once a week, once every two weeks, once every three weeks, or once every month. For example, a dosing regimen may comprise administering an initial loading dose, followed by a weekly maintenance dose of, for example, one-half of the initial dose. Or a dosing regimen may comprise administering an initial loading dose, followed by maintenance doses of, for example one-half of the initial dose every other week. Or a dosing regimen may comprise administering three initial doses for 3 weeks, followed by maintenance doses of, for example, the same amount every other week.

As is known to those of skill in the art, administration of any therapeutic agent may lead to side effects and/or toxicities. In some cases, the side effects and/or toxicities are so severe as to preclude administration of the particular agent at a therapeutically effective dose. In some cases, drug therapy must be discontinued, and other agents may be tried. However, many agents in the same therapeutic class often display similar side effects and/or toxicities, meaning that the patient either has to stop therapy, or if possible, suffer from the unpleasant side effects associated with the therapeutic agent.

The present invention provides methods of treating cancer in a subject comprising using a dosing strategy for administering two or more agents that may reduce side effects and/or toxicities associated with administration of a Wnt pathway inhibitor and/or an immunotherapeutic agent. In some embodiments, a method for treating cancer in a human subject comprises administering to the subject a therapeutically effective dose of a Wnt pathway inhibitor in combination with a therapeutically effective dose of an immunotherapeutic agent, where one or both of the inhibitors are administered according to an intermittent dosing strategy. In some embodiments, the intermittent dosing strategy comprises administering an initial dose of a Wnt pathway inhibitor to the subject, and administering subsequent doses of the Wnt pathway inhibitor about once every 2 weeks. In some embodiments, the intermittent dosing strategy comprises administering an initial dose of a Wnt pathway inhibitor to the subject, and administering subsequent doses of the Wnt pathway inhibitor about once every 3 weeks. In some embodiments, the intermittent dosing strategy comprises administering an initial dose of a Wnt pathway inhibitor to the subject, and administering subsequent doses of the Wnt pathway inhibitor about once every 4 weeks. In some embodiments, the Wnt pathway inhibitor is administered using an intermittent dosing strategy and the immunotherapeutic agent is administered weekly or every week for 3 weeks out of 4.

Combination therapy with two or more therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects and/or increasing the therapeutic index of the agent(s). Combination therapy may decrease the likelihood that resistant cancer cells will develop. Combination therapy comprising an immunotherapeutic agent may allow one agent to enhance the immune response to a tumor or tumor cells while the other agent may be effective at killing tumor cells more directly.

In some embodiments, the combination of a Wnt pathway inhibitor and an immunotherapeutic agent results in additive or synergetic results. In some embodiments, the combination therapy results in an increase in the therapeutic index of the Wnt pathway inhibitor. In some embodiments, the combination therapy results in an increase in the therapeutic index of the immunotherapeutic agent. In some embodiments, the combination therapy results in a decrease in the toxicity and/or side effects of the Wnt pathway inhibitor. In some embodiments, the combination therapy results in a decrease in the toxicity and/or side effects of the immunotherapeutic agent.

The treating physician can estimate repetition rates for dosing based on measured residence times and concentrations of the drug in bodily fluids or tissues. The progress of therapy can be monitored by conventional techniques and assays.

In certain embodiments, in addition to administering a Wnt pathway inhibitor in combination with an immunotherapeutic agent, treatment methods may further comprise administering at least one additional therapeutic agent prior to, concurrently with, and/or subsequently to administration of the Wnt pathway inhibitor and/or the immunotherapeutic agent.

In some embodiments, the additional therapeutic agent(s) will be administered substantially simultaneously or concurrently with the Wnt pathway inhibitor or the immunotherapeutic agent. For example, a subject may be given the Wnt pathway inhibitor and the immunotherapeutic agent while undergoing a course of treatment with the additional therapeutic agent (e.g., additional chemotherapeutic agent). In certain embodiments, the Wnt pathway inhibitor and the immunotherapeutic agent will be administered within 1 year of the treatment with the additional therapeutic agent. In certain alternative embodiments, the Wnt pathway inhibitor and the immunotherapeutic agent will be administered within 10, 8, 6, 4, or 2 months of any treatment with the additional therapeutic agent. In certain other embodiments, the Wnt pathway inhibitor and the immunotherapeutic agent will be administered within 4, 3, 2, or 1 week of any treatment with the additional therapeutic agent. In some embodiments, the Wnt pathway inhibitor and the immunotherapeutic agent will be administered within 5, 4, 3, 2, or 1 days of any treatment with the additional therapeutic agent. It will further be appreciated that the agents or treatment may be administered to the subject within a matter of hours or minutes (i.e., substantially simultaneously) with the Wnt pathway inhibitor or the immunotherapeutic agent.

Therapeutic agents that may be administered in combination with a Wnt pathway inhibitor and an immunotherapeutic agent include chemotherapeutic agents. Thus, in some embodiments, the method or treatment involves the administration of a Wnt pathway inhibitor and immunotherapeutic agent of the present invention in combination with a chemotherapeutic agent or cocktail of multiple different chemotherapeutic agents. Treatment with a Wnt pathway inhibitor and immunotherapeutic agent can occur prior to, concurrently with, or subsequent to administration of chemotherapies. Preparation and dosing schedules for such chemotherapeutic agents can be used according to manufacturers' instructions or as determined empirically by the skilled practitioner. Preparation and dosing schedules for such chemotherapy are also described in Chemotherapy Service, 1992, M. C. Perry, Editor, Williams & Wilkins, Baltimore, Md.

Chemotherapeutic agents useful in the instant invention include, but are not limited to, alkylating agents such as thiotepa and cyclosphosphamide; alkyl sulfonates such as busulfan, improsulfan, and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK.; razoxane; sizofuran; spirogermanium; tenuazonic acid; triaziquone; 2,′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; chlorambucil; gemcitabine; 6-thioguanine; mercaptopurine; methotrexate; platinum analogs such as cisplatin and carboplatin; platinum; etoposide (VP-16); ifosfamide; mitomycin C; mitoxantrone; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene; and antiandrogens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCl, daunorubicin citrate, mitoxantrone HCl, actinomycin D, etoposide, topotecan HCl, teniposide (VM-26), and irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, thioguanine, 5-azacytidine, 6-mercaptopurine, azathioprine, 6-thioguanine, pentostatin, fludarabine phosphate, and cladribine, as well as pharmaceutically acceptable salts, acids, or derivatives of any of these.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE®), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1.

In some embodiments, an additional therapeutic agent that may be administered in combination with a Wnt pathway inhibitor and an immunotherapeutic agent comprises an agent such as a small molecule. For example, treatment can involve the combined administration of a Wnt pathway inhibitor and an immunotherapeutic agent with a small molecule that acts as an inhibitor against tumor-associated antigens including, but not limited to, EGFR, HER2 (ErbB2), and/or VEGF. In some embodiments, a Wnt pathway inhibitor and an immunotherapeutic agent are administered in combination with a protein kinase inhibitor selected from the group consisting of: gefitinib (IRESSA), erlotinib (TARCEVA), sunitinib (SUTENT), lapatanib, vandetanib (ZACTIMA), AEE788, CI-1033, cediranib (RECENTIN), sorafenib (NEXAVAR), and pazopanib (GW786034B). In some embodiments, an additional therapeutic agent comprises an mTOR inhibitor.

In some embodiments, an additional therapeutic agent comprises a biological molecule, such as an antibody. For example, treatment can involve the combined administration of a Wnt pathway inhibitor and an immunotherapeutic agent with antibodies against tumor-associated antigens including, but not limited to, antibodies that bind EGFR, HER2/ErbB2, and/or VEGF. In certain embodiments, the additional therapeutic agent is an antibody specific for a cancer stem cell marker. In certain embodiments, the additional therapeutic agent is an antibody that inhibits a cancer stem cell pathway. In certain embodiments, the additional therapeutic agent is an antibody that is an angiogenesis inhibitor (e.g., an anti-VEGF or VEGF receptor antibody). In certain embodiments, the additional therapeutic agent is bevacizumab (AVASTIN), ramucirumab, trastuzumab (HERCEPTIN), pertuzumab (OMNITARG), panitumumab (VECTIBIX), nimotuzumab, zalutumumab, or cetuximab (ERBITUX).

Furthermore, treatment can involve the combined administration of a Wnt pathway inhibitor and an immunotherapeutic agent with other biologic molecules, such as one or more cytokines (e.g., lymphokines, interleukins, tumor necrosis factors, and/or growth factors) or can be accompanied by surgical removal of tumors, removal of cancer cells, or any other therapy deemed necessary by a treating physician.

In some embodiments, treatment can involve the combined administration of a Wnt pathway inhibitor and an immunotherapeutic agent with a growth factor selected from the group consisting of, but not limited to: adrenomedullin (AM), angiopoietin (Ang), BMPs, BDNF, EGF, erythropoietin (EPO), FGF, GDNF, G-CSF, GM-CSF, GDF9, HGF, HDGF, IGF, migration-stimulating factor, myostatin (GDF-8), NGF, neurotrophins, PDGF, thrombopoietin, TGF-α, TGF-β, TNF-α, VEGF, INGF, IL-1, IL-2, IL-3, IL-4, IL-5, IL-6, IL-7, IL-12, IL-15, and IL-18.

In certain embodiments, treatment can involve the combined administration of a Wnt pathway inhibitor and an immunotherapeutic agent with radiation therapy. Treatment with a Wnt pathway inhibitor and an immunotherapeutic agent can occur prior to, concurrently with, or subsequent to administration of radiation therapy. Dosing schedules for such radiation therapy can be determined by the skilled medical practitioner.

### III. Wnt Pathway Inhibitors

The present invention provides, in part, methods that use a Wnt pathway inhibitor in combination with an immunotherapeutic agent for treatment of patients with cancers or tumors. As used herein “Wnt pathway inhibitor” includes, but is not limited to, Frizzled (FZD) binding agents and Wnt-binding agents. FZD-binding agents may include antibodies that specifically bind to FZD proteins. Wnt-binding agents may include antibodies that specifically bind to Wnt proteins, as well as soluble FZD receptors that bind to Wnt proteins.

In certain embodiments, the Wnt pathway inhibitor acts on Wnt or on a target downstream of Wnt in the Wnt pathway. In certain embodiments, the Wnt pathway inhibitor does not interact with targets involved in Wnt secretion (e.g., porcupine or Wntless).

In certain embodiments, the Wnt pathway inhibitors are agents that bind one or more human FZD proteins. In some embodiments, the FZD-binding agents specifically bind one, two, three, four, five, six, seven, eight, nine, or ten FZD proteins. In some embodiments, the FZD-binding agent binds one or more FZD proteins selected from the group consisting of FZD1, FZD2, FZD3, FZD4, FZD5, FZD6, FZD7, FZD8, FZD9, and FZD10. In some embodiments, FZD-binding agent binds one or more FZD proteins comprising FZD1, FZD2, FZD5, FZD7, and/or FZD8. In certain embodiments, FZD-binding agent binds FZD7. In certain embodiments, FZD-binding agent binds FZD5 and/or FZD8. In certain embodiments, the FZD-binding agent specifically binds FZD1, FZD2, FZD5, FZD7, and FZD8. Non-limiting examples of FZD-binding agents can be found in U.S. Pat. No. 7,982,013.

In certain embodiments, the FZD-binding agent is a FZD antagonist. In certain embodiments, the FZD-binding agent is a Wnt pathway antagonist. In certain embodiments, the FZD-binding agent inhibits Wnt signaling. In some embodiments, the FZD-binding agent inhibits canonical Wnt signaling.

In some embodiments, the FZD-binding agents are antibodies. In some embodiments, the FZD-binding agents are polypeptides. In certain embodiments, the FZD-binding agent is an antibody or a polypeptide comprising an antigen-binding site. In certain embodiments, an antigen-binding site of a FZD-binding antibody or polypeptide described herein is capable of binding (or binds) one, two, three, four, five, or more human FZD proteins. In certain embodiments, an antigen-binding site of the FZD-binding antibody or polypeptide is capable of specifically binding one, two, three, four, or five human FZD proteins selected from the group consisting of FZD1, FZD2, FZD3, FZD4, FZD5, FZD6, FZD7, FZD8, FZD9 and FZD10. In some embodiments, when the FZD-binding agent is an antibody that binds more than one FZD protein, it may be referred to as a “pan-FZD antibody”.

In certain embodiments, the FZD-binding agent (e.g., antibody) specifically binds the extracellular domain (ECD) of the one or more human FZD proteins to which it binds. In certain embodiments, the FZD-binding agent specifically binds within the Fri domain (also known as the cysteine-rich domain (CRD)) of the human FZD protein to which it binds. Sequences of the Fri domain of each of the human FZD proteins are known in the art and are provided as SEQ ID NO:13 (FZD1), SEQ ID NO:14 (FZD2), SEQ ID NO:15 (FZD3), SEQ ID NO:16 (FZD4), SEQ ID NO:17 (FZD5), SEQ ID NO:18 (FZD6), SEQ ID NO:19 (FZD7), SEQ ID NO:20 (FZD), SEQ ID NO:21 (FZD9), and SEQ ID NO:22 (FZD 10).

In certain embodiments, the FZD-binding agent binds one, two, three, four, five, or more FZD proteins. In some embodiments, the FZD-binding agent specifically binds one, two, three, four, or five FZD proteins selected from the group consisting of FZD1, FZD2, FZD5, FZD7, and FZD8. In some embodiments, the FZD-binding agent specifically binds at least FZD5 and FZD8.

In some embodiments, the FZD-binding agent binds at least one human FZD protein with a dissociation constant (K_{D}) of about 1 μM or less, about 100 nM or less, about 40 nM or less, about 20 nM or less, about 10 nM or less, about 1 nM or less, or about 0.1 nM or less. In some embodiments, a FZD-binding agent binds at least one FZD protein with a K_{D} of about 10 nM or less. In some embodiments, a FZD-binding agent binds at least one FZD protein with a K_{D} of about 1 nM or less. In some embodiments, a FZD-binding agent binds at least one FZD protein with a K_{D} of about 0.1 nM or less. In certain embodiments, a FZD-binding agent binds each of one or more (e.g., 1, 2, 3, 4, or 5) of FZD1, FZD2, FZD5, FZD7, and FZD8 with a K_{D} of about 40 nM or less. In certain embodiments, the FZD-binding agent binds to each of one or more of FZD1, FZD2, FZD5, FZD7, and FZD8 with a K_{D} of about 10 nM or less. In certain embodiments, the FZD-binding agent binds each of FZD1, FZD2, FZD5, FZD7, and FZD8 with a K_{D} of about 10 nM. In some embodiments, the K_{D} of the binding agent (e.g., an antibody) to a FZD protein is the K_{D} determined using a FZD-Fc fusion protein comprising at least a portion of the FZD extracellular domain or FZD-Fri domain immobilized on a Biacore chip.

In certain embodiments, the FZD-binding agent binds one or more (for example, two or more, three or more, or four or more) human FZD proteins with an EC₅₀ of about 1 μM or less, about 100 nM or less, about 40 nM or less, about 20 nM or less, about 10 nM or less, or about 1 nM or less. In certain embodiments, a FZD-binding agent binds to more than one FZD protein with an EC₅₀ of about 40 nM or less, about 20 nM or less, or about 10 nM or less. In certain embodiments, the FZD-binding agent has an EC₅₀ of about 20 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 10 nM or less with respect to one or more (e.g., 1, 2, 3, 4, or 5) of the following FZD proteins: FZD1, FZD2, FZD5, FZD7, and FZD8. In certain embodiments, the FZD-binding agent has an EC₅₀ of about 40 nM or less or 20 nM or less with respect to binding of FZD5 and/or FZD8.

In certain embodiments, the Wnt pathway inhibitor is a FZD-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, or bivalent. In some embodiments, the antibody is a bispecific antibody or a multispecific antibody. In some embodiments, the antibody is conjugated to a cytotoxic moiety. In some embodiments, the antibody is isolated. In some embodiments, the antibody is substantially pure.

The FZD-binding agents (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, and protein A immunoassays. Such assays are routine and well-known in the art (see, e.g., Ausubel et al., Editors, 1994-present, Current Protocols in Molecular Biology, John Wiley & Sons, Inc., New York, N.Y.).

In certain embodiments, the invention provides a Wnt pathway inhibitor which is a FZD-binding agent (e.g., an antibody) that comprises a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3). In some embodiments, the FZD-binding agent further comprises a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In some embodiments, the FZD-binding agent comprises a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6). In certain embodiments, the FZD-binding agent comprises: (a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and (b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

In certain embodiments, the invention provides a FZD-binding agent (e.g., an antibody) that comprises: (a) a heavy chain CDR1 comprising GFTFSHYTLS (SEQ ID NO:1), or a variant thereof comprising 1, 2, 3, or 4 amino acid substitutions; (b) a heavy chain CDR2 comprising VISGDGSYTYYADSVKG (SEQ ID NO:2), or a variant thereof comprising 1, 2, 3, or 4 amino acid substitutions; (c) a heavy chain CDR3 comprising NFIKYVFAN (SEQ ID NO:3), or a variant thereof comprising 1, 2, 3, or 4 amino acid substitutions; (d) a light chain CDR1 comprising SGDNIGSFYVH (SEQ ID NO:4), or a variant thereof comprising 1, 2, 3, or 4 amino acid substitutions; (e) a light chain CDR2 comprising DKSNRPSG (SEQ ID NO:5), or a variant thereof comprising 1, 2, 3, or 4 amino acid substitutions; and (f) a light chain CDR3 comprising QSYANTLSL (SEQ ID NO:6), or a variant thereof comprising 1, 2, 3, or 4 amino acid substitutions. In certain embodiments, the amino acid substitutions are conservative substitutions.

In certain embodiments, the invention provides a FZD-binding agent (e.g., an antibody) that comprises a heavy chain variable region having at least about 80% sequence identity to SEQ ID NO:7, and/or a light chain variable region having at least 80% sequence identity to SEQ ID NO:8. In certain embodiments, the FZD-binding agent comprises a heavy chain variable region having at least about 85%, at least about 90%, at least about 95%, at least about 97%, or at least about 99% sequence identity to SEQ ID NO:7. In certain embodiments, the FZD-binding agent comprises a light chain variable region having at least about 85%, at least about 90%, at least about 95%, at least about 97%, or at least about 99% sequence identity to SEQ ID NO:8. In certain embodiments, the FZD-binding agent comprises a heavy chain variable region having at least about 95% sequence identity to SEQ ID NO:7, and/or a light chain variable region having at least about 95% sequence identity to SEQ ID NO:8. In certain embodiments, the FZD-binding agent comprises a heavy chain variable region comprising SEQ ID NO:7 and/or a light chain variable region comprising SEQ ID NO:8. In certain embodiments, the FZD-binding agent comprises a heavy chain variable region comprising SEQ ID NO:7 and a light chain variable region comprising SEQ ID NO:8. In certain embodiments, the FZD-binding agent comprises a heavy chain variable region consisting essentially of SEQ ID NO:7 and a light chain variable region consisting essentially of SEQ ID NO:8.

In certain embodiments, the invention provides a FZD-binding agent (e.g., an antibody) that comprises: (a) a heavy chain having at least 90% sequence identity to SEQ ID NO:9 (with or without the signal sequence) or SEQ ID NO:11; and/or (b) a light chain having at least 90% sequence identity to SEQ ID NO:10 (with or without the signal sequence) or SEQ ID NO:12. In some embodiments, the FZD-binding agent comprises: (a) a heavy chain having at least 95% sequence identity to SEQ ID NO:9 (with or without the signal sequence) or SEQ ID NO:11; and/or (b) a light chain having at least 95% sequence identity to SEQ ID NO:10 (with or without the signal sequence) or SEQ ID NO:12. In some embodiments, the FZD-binding agent comprises a heavy chain comprising SEQ ID NO:9 (with or without the signal sequence) or SEQ ID NO:11, and/or a light chain comprising SEQ ID NO:10 (with or without the signal sequence) or SEQ ID NO:12. In some embodiments, the FZD-binding agent comprises a heavy chain comprising SEQ ID NO:11 and a light chain comprising SEQ ID NO:12. In some embodiments, the FZD-binding agent comprises a heavy chain consisting essentially of amino acids 20-463 of SEQ ID NO:9 and a light chain consisting essentially of amino acids 20-232 of SEQ ID NO:10. In some embodiments, the FZD-binding agent comprises a heavy chain consisting essentially of SEQ ID NO:11 and a light chain consisting essentially of SEQ ID NO:12.

In certain embodiments, the invention provides a Wnt pathway inhibitor which is a FZD-binding agent (e.g., an antibody) that specifically binds at least one of FZD1, FZD2, FZD5, FZD7, and/or FZD8, wherein the FZD-binding agent (e.g., an antibody) comprises one, two, three, four, five, and/or six of the CDRs of antibody OMP-18R5. Antibody OMP-18R5 (also known as 18R5 and vantictumab), as well as other FZD-binding agents, has been previously described in U.S. Pat. No. 7,982,013. DNA encoding the heavy chain and light chain of the OMP-18R5 IgG2 antibody was deposited with the ATCC, under the conditions of the Budapest Treaty on Sep. 29, 2008, and assigned ATCC deposit designation number PTA-9541. In some embodiments, the FZD-binding agent comprises one or more of the CDRs of OMP-18R5, two or more of the CDRs of OMP-18R5, three or more of the CDRs of OMP-18R5, four or more of the CDRs of OMP-18R5, five or more of the CDRs of OMP-18R5, or all six of the CDRs of OMP-18R5.

The invention provides polypeptides which are Wnt pathway inhibitors. The polypeptides include, but are not limited to, antibodies that specifically bind human FZD proteins. In some embodiments, a polypeptide binds one or more FZD proteins selected from the group consisting of FZD1, FZD2, FZD3, FZD4, FZD5, FZD6, FZD7, FZD8, FZD9, and FZD10. In some embodiments, a polypeptide binds FZD1, FZD2, FZD5, FZD7, and/or FZD8. In some embodiments, a polypeptide binds FZD1, FZD2, FZD5, FZD7, and FZD8.

In certain embodiments, a polypeptide comprises one, two, three, four, five, and/or six of the CDRs of antibody OMP-18R5. In some embodiments, a polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds one or more human FZD proteins, wherein the polypeptide comprises an amino acid sequence having at least about 80% sequence identity to SEQ ID NO:7, and/or an amino acid sequence having at least about 80% sequence identity to SEQ ID NO:8. In certain embodiments, the polypeptide comprises an amino acid sequence having at least about 85%, at least about 90%, at least about 95%, at least about 97%, or at least about 99% sequence identity to SEQ ID NO:7. In certain embodiments, the polypeptide comprises an amino acid sequence having at least about 85%, at least about 90%, at least about 95%, at least about 97%, or at least about 99% sequence identity to SEQ ID NO:8. In certain embodiments, the polypeptide comprises an amino acid sequence having at least about 95% sequence identity to SEQ ID NO:7, and/or an amino acid sequence having at least about 95% sequence identity to SEQ ID NO:8. In certain embodiments, the polypeptide comprises an amino acid sequence comprising SEQ ID NO:7, and/or an amino acid sequence comprising SEQ ID NO:8.

In some embodiments, a FZD-binding agent comprises a polypeptide comprising a sequence selected from the group consisting of: SEQ ID NO:7, SEQ ID NO:8, SEQ ID NO:9, SEQ ID NO:10, SEQ ID NO:11, and SEQ ID NO:12.

In certain embodiments, a FZD-binding agent comprises the heavy chain variable region and light chain variable region of the OMP-18R5 antibody. In certain embodiments, a FZD-binding agent comprises the heavy chain and light chain of the OMP-18R5 antibody (with or without the leader sequence).

In certain embodiments, a FZD-binding agent comprises, consists essentially of, or consists of, the antibody OMP-18R5.

In certain embodiments, a FZD-binding agent (e.g., antibody) competes for specific binding to one or more human FZD proteins with an antibody that comprises a heavy chain variable region comprising SEQ ID NO:7 and a light chain variable region comprising SEQ ID NO:8. In certain embodiments, a FZD-binding agent (e.g., antibody) competes for specific binding to one or more human FZD proteins with an antibody that comprises a heavy chain comprising SEQ ID NO:9 (with or without the signal sequence) and a light chain comprising SEQ ID NO:10 (with or without the signal sequence). In certain embodiments, a FZD-binding agent (e.g., antibody) competes for specific binding to one or more human FZD proteins with an antibody that comprises a heavy chain comprising SEQ ID NO:11 and a light chain comprising SEQ ID NO:12. In certain embodiments, a FZD-binding agent competes with antibody OMP-18R5 for specific binding to one or more human FZD proteins. In some embodiments, a FZD-binding agent or antibody competes for specific binding to one or more human FZD proteins in an in vitro competitive binding assay.

In certain embodiments, a FZD-binding agent (e.g., an antibody) binds the same epitope, or essentially the same epitope, on one or more human FZD proteins as an antibody of the invention. In another embodiment, a FZD-binding agent is an antibody that binds an epitope on one or more human FZD proteins that overlaps with the epitope on a FZD protein bound by an antibody of the invention. In certain embodiments, a FZD-binding agent (e.g., an antibody) binds the same epitope, or essentially the same epitope, on one or more FZD proteins as antibody OMP-18R5. In another embodiment, the FZD-binding agent is an antibody that binds an epitope on one or more human FZD proteins that overlaps with the epitope on a FZD protein bound by antibody OMP-18R5.

In certain embodiments, the Wnt pathway inhibitors are agents that bind one or more human Wnt proteins. In certain embodiments, the agents specifically bind one, two, three, four, five, six, seven, eight, nine, ten, or more Wnt proteins. In some embodiments, the Wnt-binding agents bind one or more human Wnt proteins selected from the group consisting of Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In certain embodiments, a Wnt-binding agent binds one or more (or two or more, three or more, four or more, five or more, etc.) Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the one or more (or two or more, three or more, four or more, five or more, etc.) Wnt proteins are selected from the group consisting of Wnt1, Wnt2, Wnt2b, Wnt3, Wnt3a, Wnt8a, Wnt8b, Wnt10a, and Wnt10b.

In certain embodiments, the Wnt-binding agent is a Wnt antagonist. In certain embodiments, the Wnt-binding agent is a Wnt pathway antagonist. In certain embodiments, the Wnt-binding agent inhibits Wnt signaling. In some embodiments, the Wnt-binding agent inhibits canonical Wnt signaling.

In some embodiments, the Wnt-binding agent is an antibody. In some embodiments, the Wnt-binding agent is a polypeptide. In certain embodiments, the Wnt-binding agent is an antibody or a polypeptide comprising an antigen-binding site. In certain embodiments, an antigen-binding site of a Wnt-binding antibody or polypeptide described herein is capable of binding (or binds) one, two, three, four, five, or more human Wnt proteins. In certain embodiments, an antigen-binding site of the Wnt-binding antibody or polypeptide is capable of specifically binding one, two, three, four, or five human Wnt proteins selected from the group consisting of Wnt1, Wnt2, Wnt2b, Wnt3, Wnt3a, Wnt7a, Wnt7b, Wnt8a, Wnt8b, Wnt10a, and Wnt10b. Non-limiting examples of Wnt-binding agents can be found in International Publication WO 2011/088127.

In certain embodiments, a Wnt-binding agent binds to the C-terminal cysteine rich domain of one or more human Wnt proteins. In certain embodiments, the Wnt-binding agent binds a domain within the one or more Wnt proteins selected from the group consisting of: SEQ ID NO:46 (Wnt1), SEQ ID NO:47 (Wnt2), SEQ ID NO:48 (Wnt2b), SEQ ID NO:49 (Wnt3), SEQ ID NO:50 (Wnt3a), SEQ ID NO:51 (Wnt7a), SEQ ID NO:52 (Wnt7b), SEQ ID NO:53 (Wnt8a), SEQ ID NO:54 (Wnt8b), SEQ ID NO:55 (Wnt10a), and SEQ ID NO:56 (Wnt10b).

In certain embodiments, the Wnt-binding agent binds one or more (e.g., two or more, three or more, or four or more) Wnt proteins with a K_{D} of about 1 μM or less, about 100 nM or less, about 40 nM or less, about 20 nM or less, or about 10 nM or less. For example, in certain embodiments, a Wnt-binding agent described herein that binds more than one Wnt protein, binds those Wnt proteins with a K_{D} of about 100 nM or less, about 20 nM or less, or about 10 nM or less. In certain embodiments, the Wnt-binding agent binds each of one or more (e.g., 1, 2, 3, 4, or 5) Wnt proteins with a K_{D} of about 40 nM or less, wherein the Wnt proteins are selected from the group consisting of: Wnt1, Wnt2, Wnt2b, Wnt3, Wnt3a, Wnt7a, Wnt7b, Wnt8a, Wnt8b, Wnt10a, and Wnt10b. In some embodiments, the K_{D} of the binding agent (e.g., an antibody) to a Wnt protein is the K_{D} determined using a Wnt fusion protein comprising at least a portion of the Wnt C-terminal cysteine rich domain immobilized on a Biacore chip.

In certain embodiments, the Wnt-binding agent binds one or more (for example, two or more, three or more, or four or more) human Wnt proteins with an EC₅₀ of about 1 μM or less, about 100 nM or less, about 40 nM or less, about 20 nM or less, about 10 nM or less, or about 1 nM or less. In certain embodiments, a Wnt-binding agent binds to more than one Wnt with an EC₅₀ of about 40 nM or less, about 20 nM or less, or about 10 nM or less. In certain embodiments, the Wnt-binding agent has an EC₅₀ of about 20 nM or less with respect to one or more (e.g., 1, 2, 3, 4, or 5) of Wnt proteins Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and/or Wnt16. In certain embodiments, the Wnt-binding agent has an EC₅₀ of about 10 nM or less with respect to one or more (e.g., 1, 2, 3, 4, or 5) of the following Wnt proteins Wnt1, Wnt2, Wnt2b, Wnt3, Wnt3a, Wnt8a, Wnt8b, Wnt10a, and/or Wnt10b.

In certain embodiments, the Wnt pathway inhibitor is a Wnt-binding agent which is an antibody. In some embodiments, the antibody is a recombinant antibody. In some embodiments, the antibody is a monoclonal antibody. In some embodiments, the antibody is a chimeric antibody. In some embodiments, the antibody is a humanized antibody. In some embodiments, the antibody is a human antibody. In certain embodiments, the antibody is an IgG1 antibody. In certain embodiments, the antibody is an IgG2 antibody. In certain embodiments, the antibody is an antibody fragment comprising an antigen-binding site. In some embodiments, the antibody is monovalent, monospecific, or bivalent. In some embodiments, the antibody is a bispecific antibody or a multispecific antibody. In some embodiments, the antibody is conjugated to a cytotoxic moiety. In some embodiments, the antibody is isolated. In some embodiments, the antibody is substantially pure.

The Wnt-binding agents (e.g., antibodies) of the present invention can be assayed for specific binding by any method known in the art as described herein for FZD-binding agents.

In certain embodiments, the Wnt-binding agent is a soluble receptor. In certain embodiments, the Wnt-binding agent comprises the extracellular domain of a FZD receptor protein. In some embodiments, the Wnt-binding agent comprises a Fri domain of a FZD protein. In some embodiments, a soluble receptor comprising a FZD Fri domain can demonstrate altered biological activity (e.g., increased protein half-life) compared to a soluble receptor comprising the entire FZD ECD. Protein half-life can be further modified (i.e., increased) by covalent modification with polyethylene glycol (PEG) or polyethylene oxide (PEO). In certain embodiments, the FZD protein is a human FZD protein. In certain embodiments, the human FZD protein is FZD1, FZD2, FZD3, FZD4, FZD5, FZD6, FZD7, FZD8, FZD9, or FZD10. Non-limiting examples of soluble FZD receptors can be found in U.S. Pat. Nos. 7,723,477 and 7,947,277 and U.S. Patent Publication No. 2013/0034551.

The predicted Fri domains for each of the human FZD1-10 proteins are provided as SEQ ID NOs:13-22. The predicted minimal Fri domains for each of the human FZD1-10 proteins are provided as SEQ ID NOs:23-32. Those of skill in the art may differ in their understanding of the exact amino acids corresponding to the various Fri domains. Thus, the N-terminus and/or C-terminus of the domains outlined above and herein may extend or be shortened by 1, 2, 3, 4, 5, 6, 7, 8, 9, or even 10 amino acids.

In certain embodiments, the Wnt-binding agent comprises a Fri domain of a human FZD protein, or a fragment or variant of the Fri domain that binds one or more human Wnt proteins. In certain embodiments, the human FZD protein is FZD1, FZD2, FZD3, FZD4, FZD5, FZD6, FZD7, FZD8, FZD9, or FZD10. In certain embodiments, the human FZD protein is FZD4. In certain embodiments, the human FZD protein is FZD5. In certain embodiments, the human FZD protein is FZD8. In certain embodiments, the human FZD protein is FZD10. In certain embodiments, the FZD protein is FZD4 and the Wnt-binding agent comprises SEQ ID NO:16. In certain embodiments, the FZD protein is FZD5 and the Wnt-binding agent comprises SEQ ID NO:17. In certain embodiments, the FZD protein is FZD7 and the Wnt-binding agent comprises SEQ ID NO:19. In certain embodiments, the FZD protein is FZD8 and the Wnt-binding agent comprises SEQ ID NO:20. In certain embodiments, the FZD protein is FZD10 and the Wnt-binding agent comprises SEQ ID NO:22. In certain embodiments, the FZD protein is FZD8 and the Wnt-binding agent comprises SEQ ID NO:33.

In some embodiments, the Wnt-binding agent comprises a Fri domain comprising the minimal Fri domain of FZD1 (SEQ ID NO:23), the minimal Fri domain of FZD2 (SEQ ID NO:24), the minimal Fri domain of FZD3 (SEQ ID NO:25), the minimal Fri domain of FZD4 (SEQ ID NO:26), the minimal Fri domain of FZD5 (SEQ ID NO:27), the minimal Fri domain of FZD6 (SEQ ID NO:28), the minimal Fri domain of FZD7 (SEQ ID NO:29), the minimal Fri domain of FZD8 (SEQ ID NO:30), the minimal Fri domain of FZD9 (SEQ ID NO:31), or the minimal Fri domain of FZD10 (SEQ ID NO:32). In some embodiments, the Wnt-binding agent comprises a Fri domain comprising the minimal Fri domain of FZD8 (SEQ ID NO:30).

In some embodiments, the Wnt-binding agent comprises a Fri domain consisting essentially of the Fri domain of FZD1, the Fri domain of FZD2, the Fri domain of FZD3, the Fri domain of FZD4, the Fri domain of FZD5, the Fri domain of FZD6, the Fri domain of FZD7, the Fri domain of FZD8, the Fri domain of FZD9, or the Fri domain of FZD10. In some embodiments, the Wnt-binding agent comprises a Fri domain consisting essentially of the Fri domain of FZD8.

In some embodiments, the Wnt-binding agent comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and SEQ ID NO:33. In some embodiments, the Wnt-binding agent comprises a Fri domain consisting essentially of SEQ ID NO:20. In some embodiments, the Wnt-binding agent comprises a Fri domain consisting essentially of SEQ ID NO:33.

In certain embodiments, the Wnt-binding agent comprises a variant of any one of the aforementioned FZD Fri domain sequences that comprises one or more (e.g., one, two, three, four, five, six, seven, eight, nine, ten, etc.) conservative substitutions and is capable of binding Wnt protein(s).

In certain embodiments, a Wnt-binding agent, such as an agent comprising a Fri domain of a human FZD receptor, further comprises a non-FZD polypeptide. In some embodiments, a FZD soluble receptor may include FZD ECD or Fri domains linked to other non-FZD functional and structural polypeptides including, but not limited to, a human Fc region, protein tags (e.g., myc, FLAG, GST), other endogenous proteins or protein fragments, or any other useful protein sequence including any linker region between a FZD ECD or Fri domain and a second polypeptide. In certain embodiments, the non-FZD polypeptide comprises a human Fc region. The Fc region can be obtained from any of the classes of immunoglobulin, IgG, IgA, IgM, IgD and IgE. In some embodiments, the Fc region is a human IgG1 Fc region. In some embodiments, the Fc region is a human IgG2 Fc region. In some embodiments, the Fc region is a wild-type Fc region. In some embodiments, the Fc region is a mutated Fc region. In some embodiments, the Fc region is truncated at the N-terminal end by 1, 2, 3, 4, 5, 6, 7, 8, 9, or 10 amino acids, (e.g., in the hinge domain). In some embodiments, an amino acid in the hinge domain is changed to hinder undesirable disulfide bond formation. In some embodiments, a cysteine is replaced with a serine to hinder or block undesirable disulfide bond formation. In some embodiments, the Fc region is truncated at the C-terminal end by 1, 2, 3, or more amino acids. In some embodiments, the Fc region is truncated at the C-terminal end by 1 amino acid. In certain embodiments, the non-FZD polypeptide comprises SEQ ID NO:34, SEQ ID NO:35, SEQ ID NO:36, SEQ ID NO:37, or SEQ ID NO:38. In certain embodiments, the non-FZD polypeptide consists essentially of SEQ ID NO:34, SEQ ID NO:35, SEQ ID NO:36, SEQ ID NO:37, or SEQ ID NO:38. In certain embodiments, the non-FZD polypeptide consists essentially of SEQ ID NO:36 or SEQ ID NO:37.

In certain embodiments, a Wnt-binding agent is a fusion protein comprising at least a minimal Fri domain of a FZD receptor and a Fc region. As used herein, a “fusion protein” is a hybrid protein expressed by a nucleic acid molecule comprising nucleotide sequences of at least two genes. In some embodiments, the C-terminus of the first polypeptide is linked to the N-terminus of the immunoglobulin Fc region. In some embodiments, the first polypeptide (e.g., a FZD Fri domain) is directly linked to the Fc region (i.e. without an intervening linker). In some embodiments, the first polypeptide is linked to the Fc region via a linker.

As used herein, the term “linker” refers to a linker inserted between a first polypeptide (e.g., a FZD component) and a second polypeptide (e.g., a Fc region). In some embodiments, the linker is a peptide linker. Linkers should not adversely affect the expression, secretion, or bioactivity of the polypeptide. Linkers should not be antigenic and should not elicit an immune response. Suitable linkers are known to those of skill in the art and often include mixtures of glycine and serine residues and often include amino acids that are sterically unhindered. Other amino acids that can be incorporated into useful linkers include threonine and alanine residues. Linkers can range in length, for example from 1-50 amino acids in length, 1-22 amino acids in length, 1-10 amino acids in length, 1-5 amino acids in length, or 1-3 amino acids in length. Linkers may include, but are not limited to, SerGly, GGSG, GSGS, GGGS, S(GGS)n where n is 1-7, GRA, poly(Gly), poly(Ala), ESGGGGVT (SEQ ID NO:57), LESGGGGVT (SEQ ID NO:58), GRAQVT (SEQ ID NO:59), WRAQVT (SEQ ID NO:60), and ARGRAQVT (SEQ ID NO:61). As used herein, a “linker” is an intervening peptide sequence that does not include amino acid residues from either the C-terminus of the first polypeptide (e.g., a FZD Fri domain) or the N-terminus of the second polypeptide (e.g., the Fc region).

In some embodiments, the Wnt-binding agent comprises a FZD Fri domain, a Fc region, and a linker connecting the FZD Fri domain to the Fc region. In some embodiments, the FZD Fri domain comprises SEQ ID NO:20, SEQ ID NO:30, or SEQ ID NO:33. In some embodiments, the linker comprises ESGGGGVT (SEQ ID NO:57) or LESGGGGVT (SEQ ID NO:58).

In some embodiments, the Wnt-binding agent comprises a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is directly linked to the second polypeptide. In some embodiments, the Wnt-binding agent comprises a first polypeptide comprising SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:20 and a second polypeptide comprising SEQ ID NO:36 or SEQ ID NO:37. In some embodiments, the Wnt-binding agent comprises a first polypeptide consisting essentially of SEQ ID NO:20 and a second polypeptide consisting essentially of SEQ ID NO:36 or SEQ ID NO:37. In some embodiments, the Wnt-binding agent comprises a first polypeptide comprising SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:30 and a second polypeptide comprising SEQ ID NO:36 or SEQ ID NO:37. In some embodiments, the Wnt-binding agent comprises a first polypeptide comprising SEQ ID NO:33 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:33 and a second polypeptide comprising SEQ ID NO:36, SEQ ID NO:37, or SEQ ID NO:35. In some embodiments, the Wnt-binding agent comprises a first polypeptide consisting essentially of SEQ ID NO:33 and a second polypeptide consisting essentially of SEQ ID NO:36, SEQ ID NO:37, or SEQ ID NO:35.

In some embodiments, the Wnt-binding agent comprises a first polypeptide comprising SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is connected to the second polypeptide by a linker. In some embodiments, the Wnt-binding agent comprises a first polypeptide comprising SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:20 and a second polypeptide comprising SEQ ID NO:36 or SEQ ID NO:37. In some embodiments, the Wnt-binding agent comprises a first polypeptide consisting essentially of SEQ ID NO:20 and a second polypeptide consisting essentially of SEQ ID NO:36 or SEQ ID NO:37. In some embodiments, the Wnt-binding agent comprises a first polypeptide comprising SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:33 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide comprising SEQ ID NO:33 and a second polypeptide comprising SEQ ID NO:36, SEQ ID NO:37, or SEQ ID NO:35. In some embodiments, the Wnt-binding agent comprises a first polypeptide consisting essentially of SEQ ID NO:33 and a second polypeptide consisting essentially of SEQ ID NO:36, SEQ ID NO:37, or SEQ ID NO:35.

In some embodiments, the Wnt-binding agent comprises a first polypeptide that is at least 95% identical to SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is directly linked to the second polypeptide. In some embodiments, the Wnt-binding agent comprises a first polypeptide that is at least 95% identical to SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:33 and a second polypeptide comprising SEQ ID NO:34, SEQ ID NO:35, SEQ ID NO:36, SEQ ID NO:37, or SEQ ID NO:38.

In some embodiments, the Wnt-binding agent comprises a first polypeptide that is at least 95% identical to SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and a second polypeptide comprising SEQ ID NO:34, SEQ ID NO:35, SEQ ID NO:36, SEQ ID NO:37, or SEQ ID NO:38, wherein the first polypeptide is connected to the second polypeptide by a linker. In some embodiments, the Wnt-binding agent comprises a first polypeptide that is at least 95% identical to SEQ ID NO:20 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:30 and a second polypeptide comprising SEQ ID NO:34, SEQ ID NO:35, SEQ ID NO:36, SEQ ID NO:37, or SEQ ID NO:38. In some embodiments, the Wnt-binding agent comprises a first polypeptide that is at least 95% identical to SEQ ID NO:33 and a second polypeptide comprising SEQ ID NO:34, SEQ ID NO:35, SEQ ID NO:36, SEQ ID NO:37, or SEQ ID NO:38.

FZD proteins contain a signal sequence that directs the transport of the proteins. Signal sequences (also referred to as signal peptides or leader sequences) are located at the N-terminus of nascent polypeptides. They target the polypeptide to the endoplasmic reticulum and the proteins are sorted to their destinations, for example, to the inner space of an organelle, to an interior membrane, to the cell outer membrane, or to the cell exterior via secretion. Most signal sequences are cleaved from the protein by a signal peptidase after the proteins are transported to the endoplasmic reticulum. The cleavage of the signal sequence from the polypeptide usually occurs at a specific site in the amino acid sequence and is dependent upon amino acid residues within the signal sequence. Although there is usually one specific cleavage site, more than one cleavage site may be recognized and/or used by a signal peptidase resulting in a non-homogenous N-terminus of the polypeptide. For example, the use of different cleavage sites within a signal sequence can result in a polypeptide expressed with different N-terminal amino acids. Accordingly, in some embodiments, the polypeptides described herein may comprise a mixture of polypeptides with different N-termini. In some embodiments, the N-termini differ in length by 1, 2, 3, 4, 5, 6, 7, 8, 9, 10, or more amino acids. In some embodiments, the N-termini differ in length by 1, 2, 3, 4, or 5 amino acids. In some embodiments, the polypeptide is substantially homogeneous, i.e., the polypeptides have the same N-terminus. In some embodiments, the signal sequence of the polypeptide comprises one or more (e.g., one, two, three, four, five, six, seven, eight, nine, ten, etc.) amino acid substitutions and/or deletions. In some embodiments, the signal sequence of the polypeptide comprises amino acid substitutions and/or deletions that allow one cleavage site to be dominant, thereby resulting in a substantially homogeneous polypeptide with one N-terminus.

In some embodiments, the Wnt-binding agent comprises an amino acid sequence selected from the group consisting of: SEQ ID NO:39, SEQ ID NO:40, SEQ ID NO:41, SEQ ID NO:42, SEQ ID NO:43, SEQ ID NO:44, and SEQ ID NO:45.

In certain embodiments, the Wnt-binding agent comprises the sequence of SEQ ID NO:39. In certain embodiments, the agent comprises the sequence of SEQ ID NO:39, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:39. In certain embodiments, the variants of SEQ ID NO:39 maintain the ability to bind one or more human Wnt proteins.

In certain embodiments, the Wnt-binding agent comprises the sequence of SEQ ID NO:40. In some embodiments, the Wnt-binding agent is SEQ ID NO:40. In certain alternative embodiments, the agent comprises the sequence of SEQ ID NO:40, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:40. In certain embodiments, the variants of SEQ ID NO:40 maintain the ability to bind one or more human Wnt proteins.

In certain embodiments, the Wnt-binding agent comprises the sequence of SEQ ID NO:41. In some embodiments, the Wnt-binding agent is SEQ ID NO:41. In certain alternative embodiments, the agent comprises the sequence of SEQ ID NO:41, comprising one or more (e.g., one, two, three, four, five, six, seven, eight, nine, ten, etc.) conservative substitutions. In certain embodiments, the agent comprises a sequence having at least about 90%, about 95%, or about 98% sequence identity with SEQ ID NO:41. In certain embodiments, the variants of SEQ ID NO:41 maintain the ability to bind one or more human Wnt proteins.

In some embodiments, the Wnt-binding agent is OMP-54F28.

In certain embodiments, a Wnt-binding agent is a polypeptide comprising an amino acid sequence selected from the group consisting of: SEQ ID NO:39, SEQ ID NO:40, SEQ ID NO:41, SEQ ID NO:42, SEQ ID NO:43, SEQ ID NO:44, and SEQ ID NO:45. In certain embodiments, the polypeptide comprises an amino acid sequence selected from the group consisting of SEQ ID NO:39, SEQ ID NO:40, and SEQ ID NO:41. In some embodiments, a polypeptide consists essentially of an amino acid sequence selected from the group consisting of: SEQ ID NO:39, SEQ ID NO:40, and SEQ ID NO:41. In certain embodiments, the polypeptide comprises the amino acid sequence of SEQ ID NO:39. In some embodiments, the polypeptide comprises the amino acid sequence of SEQ ID NO:40. In certain embodiments, the polypeptide comprises the amino acid sequence of SEQ ID NO:41. In certain embodiments, the polypeptide comprises the amino acid sequence of SEQ ID NO:42. In certain embodiments, the polypeptide comprises the amino acid sequence of SEQ ID NO:43. In certain embodiments, the polypeptide comprises the amino acid sequence of SEQ ID NO:44. In certain embodiments, the polypeptide comprises the amino acid sequence of SEQ ID NO:45.

In some embodiments, the polypeptide is a substantially purified polypeptide comprising an amino acid sequence selected from the group consisting of SEQ ID NO:39, SEQ ID NO:40, and SEQ ID NO:41. In some embodiments, the polypeptide is a substantially purified polypeptide comprising SEQ ID NO:41. In certain embodiments, the substantially purified polypeptide consists of at least 90% of a polypeptide that has an N-terminal sequence of ASA. In some embodiments, the nascent polypeptide comprises a signal sequence that results in a substantially homogeneous polypeptide product with one N-terminal sequence.

In certain embodiments, a Wnt-binding agent comprises a Fc region of an immunoglobulin. Those skilled in the art will appreciate that some of the binding agents of this invention will comprise fusion proteins in which at least a portion of the Fc region has been deleted or otherwise altered so as to provide desired biochemical characteristics, such as increased cancer cell localization, increased tumor penetration, reduced serum half-life, or increased serum half-life, when compared with a fusion protein of approximately the same immunogenicity comprising a native or unaltered constant region. Modifications to the Fc region may include additions, deletions, or substitutions of one or more amino acids in one or more domains. The modified fusion proteins disclosed herein may comprise alterations or modifications to one or more of the two heavy chain constant domains (CH2 or CH3) or to the hinge region. In other embodiments, the entire CH2 domain may be removed (ΔCH2 constructs). In some embodiments, the omitted constant region domain is replaced by a short amino acid spacer (e.g., 10 aa residues) that provides some of the molecular flexibility typically imparted by the absent constant region domain.

In some embodiments, the modified fusion proteins are engineered to link the CH3 domain directly to the hinge region. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the fusion protein.

In some embodiments, the modified fusion proteins may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g., complement C1q binding). Such partial deletions of the constant regions may improve selected characteristics of the binding agent (e.g., serum half-life) while leaving other desirable functions associated with the subject constant region domain intact. Moreover, as alluded to above, the constant regions of the disclosed fusion proteins may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified fusion protein. In certain embodiments, the modified fusion proteins comprise the addition of one or more amino acids to the constant region to enhance desirable characteristics such as decreasing or increasing effector function, or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an immunoglobulin can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In some embodiments, the modified fusion proteins provide for altered effector functions that, in turn, affect the biological profile of the administered agent. For example, in some embodiments, the deletion or inactivation (through point mutations or other means) of a constant region domain may reduce Fc receptor binding of the circulating modified agent, thereby increasing cancer cell localization and/or tumor penetration. In other embodiments, the constant region modifications increase or reduce the serum half-life of the agent. In some embodiments, the constant region is modified to eliminate disulfide linkages or oligosaccharide moieties.

In certain embodiments, a modified fusion protein does not have one or more effector functions normally associated with an Fc region. In some embodiments, the agent has no antibody-dependent cell-mediated cytotoxicity (ADCC) activity, and/or no complement-dependent cytotoxicity (CDC) activity. In certain embodiments, the agent does not bind to the Fc receptor and/or complement factors. In certain embodiments, the agent has no effector function.

In some embodiments, the Wnt-binding agent (e.g., a soluble receptor) described herein is modified to reduce immunogenicity. In general, immune responses against completely normal human proteins are rare when these proteins are used as therapeutics. However, although many fusion proteins comprise polypeptides sequences that are the same as the sequences found in nature, several therapeutic fusion proteins have been shown to be immunogenic in mammals. In some studies, a fusion protein comprising a linker has been found to be more immunogenic than a fusion protein that does not contain a linker. Accordingly, in some embodiments, the polypeptides of the invention are analyzed by computation methods to predict immunogenicity. In some embodiments, the polypeptides are analyzed for the presence of T-cell and/or B-cell epitopes. If any T-cell or B-cell epitopes are identified and/or predicted, modifications to these regions (e.g., amino acid substitutions) may be made to disrupt or destroy the epitopes. Various algorithms and software that can be used to predict T-cell and/or B-cell epitopes are known in the art. For example, the software programs SYFPEITHI, HLA Bind, PEPVAC, RANKPEP, DiscoTope, ElliPro, and Antibody Epitope Prediction are all publicly available.

In some embodiments, a cell producing any of the Wnt-binding agents (e.g., soluble receptors) or polypeptides described herein is provided. In some embodiments, a composition comprising any of the Wnt-binding agents (e.g., soluble receptors) or polypeptides described herein is provided. In some embodiments, the composition comprises a polypeptide wherein at least 80%, 90%, 95%, 97%, 98%, or 99% of the polypeptide has an N-terminal sequence of ASA. In some embodiments, the composition comprises a polypeptide wherein 100% of the polypeptide has an N-terminal sequence of ASA. In some embodiments, the composition comprises a polypeptide wherein at least 80% of the polypeptide has an N-terminal sequence of ASA. In some embodiments, the composition comprises a polypeptide wherein at least 90% of the polypeptide has an N-terminal sequence of ASA. In some embodiments, the composition comprises a polypeptide wherein at least 95% of the polypeptide has an N-terminal sequence of ASA.

The polypeptides described herein can be recombinant polypeptides, natural polypeptides, or synthetic polypeptides. It will be recognized in the art that some amino acid sequences of the invention can be varied without significant effect on the structure or function of the protein. If such differences in sequence are contemplated, it should be remembered that there will be critical areas on the protein which determine activity. Thus, the invention further includes variations of the polypeptides which show substantial activity or which include regions of FZD proteins, such as the protein portions discussed herein. Such mutants include deletions, insertions, inversions, repeats, and type substitutions.

Of course, the number of amino acid substitutions a skilled artisan would make depends on many factors, including those described above. In certain embodiments, the number of substitutions for any given soluble receptor polypeptide will not be more than 50, 40, 30, 25, 20, 15, 10, 5 or 3.

Fragments or portions of the polypeptides of the present invention can be employed for producing the corresponding full-length polypeptide by peptide synthesis; therefore, the fragments can be employed as intermediates for producing the full-length polypeptides. These fragments or portion of the polypeptides can also be referred to as “protein fragments” or “polypeptide fragments”.

A “protein fragment” of this invention is a portion or all of a protein which is capable of binding to one or more human Wnt proteins or one or more human FZD proteins. In some embodiments, the fragment has a high affinity for one or more human Wnt proteins. In some embodiments, the fragment has a high affinity for one or more human FZD proteins. Some fragments of Wnt-binding agents described herein are protein fragments comprising at least part of the extracellular portion of a FZD protein linked to at least part of a constant region of an immunoglobulin (e.g., a Fc region). The binding affinity of the protein fragment can be in the range of about 10⁻¹¹ to 10⁻¹²M, although the affinity can vary considerably with fragments of different sizes, ranging from 10⁻⁷ to 10⁻¹³M. In some embodiments, the fragment is about 100 to about 200 amino acids in length and comprises a binding domain linked to at least part of a constant region of an immunoglobulin.

In some embodiments, the Wnt pathway inhibitors are small molecules. Exemplary small molecules are described, for example, in PCT Publication Nos. WO 2014/147021 and WO 2014/147182, each of which is hereby incorporated by reference. PCT Publication Nos. WO 2014/147021 describes compounds of general formula:

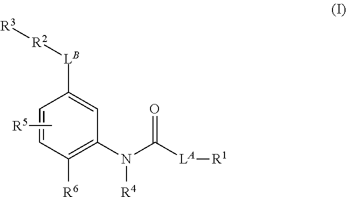

in which
L^{A} represents a methylene or ethylene group, said methylene or ethylene group being optionally substituted, one or more times, identically or differently, with a substituent selected from hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-, 3- to 10-membered heterocycloalkyl-; or, when two substituents are present at the same carbon atom, the two substituents, together with the carbon atom they are attached to, may form a C₃-C₆-cycloalkyl- or 3- to 6-membered heterocycloalkyl-ring; wherein the ring is optionally substituted one or more times, identically or differently, with a substituent selected from: halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from 5- to 8-membered heterocycloalkyl-, 4- to 10-membered heterocycloalkenyl-, aryl-, heteroaryl-, and —N(R⁷)-(d-C₆-alkyl); wherein said 5-to 8-membered heterocycloalkyl-, 4- to 10-membered heterocycloalkenyl-, aryl-, heteroaryl-, and —N(R⁷)-(Ci-C6-alkyl) group is optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-;
R² represents a group selected from:

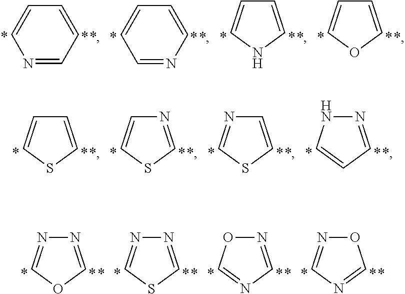

wherein “*” indicates the point of attachment to R³, and “**” indicates the point of attachment to L^{B};
wherein said group is optionally substituted, one or more times, identically or differently, with a C₁-C₃-alkyl-group;
R³ represents a phenyl group, said phenyl-group being optionally substituted, one or more times, identically or differently, with a substituent selected from halo-, hydroxy-, —N(R⁹)(R¹⁰), —N(H)C(═O)R⁹, cyano-, nitro-, C₁-C₃-alkyl-, C₁-C₃-alkoxy-, halo-C₁-C₃-alkyl-, hydroxy-C₁-C₃-alkyl-, amino-C₁-C₃-alkyl-, halo-C₁-C₃-alkoxy-;
R⁴ represents a hydrogen atom or a C₁-C₃-alkyl-group;
R⁵ represents a hydrogen atom or a halogen atom or a group selected from cyano-, C₁-C₃-alkyl-, C₁-C₃-alkoxy-;
R⁶ represents a group selected from C₁-C₆-alkyl-, C₂-C₆-alkenyl-, C₂-C₆-alkynyl-, C₁-C₆-alkoxy-, C₃-C₆-cycloalkoxy-, halo-, hydroxy-, cyano-, aryl-, heteroaryl-, —N(R⁹)(R¹⁰), —C(═O)—O—C₁-C₄-alkyl, —C(═O)—N(R⁹)(R¹⁰), R⁹—S—, R⁹—S(═O)—, R⁹—S(═O)₂—; the C₁-C₆-alkyl-, C₂-C₆-alkenyl-, C₂-C₆-alkynyl-, aryl-, heteroaryl-, and C₁-C₆-alkoxy-group being optionally substituted, one or more times, identically or differently, with a substituent selected from halo-, cyano-, nitro-, hydroxy-, C₁-C₃-alkyl-, C₁-C₃-alkoxy-, halo-C₁-C₃-alkoxy-, hydroxy-C₁-C₃-alkoxy-, C₁-C₃-alkoxy-C₂-C₃-alkoxy-, C₃-C₇-cycloalkyl-, C₄-C₇-cycloalkenyl-, 3- to 10-membered heterocycloalkyl-, 4- to 10-membered heterocycloalkenyl-, aryl-, heteroaryl-, —C(═O)R⁹, —C(═O)O—(C₁-C₄-alkyl), —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—, —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰, —S(═O)₂N(H)R⁹, —S(═O)₂NR¹⁰R⁹, —S(═O)(═NR¹⁰)R⁹, —N═S(═O)(R¹⁰)R⁹;
R⁷ represents a hydrogen atom or a C₁-C₃-alkyl- or C₁-C₃-alkoxy-C₁-C₃-alkyl-group;
R⁹, R¹⁰, R¹¹ represent, independently, a hydrogen atom or a C₁-C₃-alkyl- or C₁-C₃-alkoxy-C₁-C₃-alkyl-group; or
R⁹R¹⁰ together with the atom or the group of atoms they are attached to, form a 3- to 10-membered heterocycloalkyl- or 4- to 10-membered heterocycloalkenyl-group;
or a tautomer, an N-oxide, a hydrate, a solvate, or a salt thereof, or a mixture of same.

In certain embodiments, the compound described in PCT Publication WO 2014/147021 is described in any of Examples 1-294. In particular embodiments, the compound is one of those listed in Table 3 of WO 2014/147021, i.e., is selected from the group consisting of N-[6-(2-fluorophenyl)pyridin-3-yl]-4-methoxy-3-[(morpholin-4-ylacetyl)amino]benzamideN-[6-(2-fluorophenyl)pyridin-3-yl]-4-methoxy-3-[(8-oxa-3-azabicyclo[3.2.1]oct-3-ylacetyl)amino] benzamide, N-[6-(2-fluorophenyl)pyridin-3-yl]-3-[(8-oxa-3-azabicyclo[3.2.1]oct-3-ylacetyl)amino]-4-(trifluoromethoxy)benzamide, N-[6-(2-fluorophenyl)pyridin-3-yl]-3-{[2-(morpholin-4-yl)propanoyl]amino}-4-(trifluoromethoxy)benzamide, N-[6-(2-fluorophenyl)pyridin-3-yl]-3-{[(2S)-2-(morpholin-4-yl)propanoyl]amino}-4-(trifluoromethoxy)benzamide, N-[6-(2-fluorophenyl)pyridin-3-yl]-3-{[(2R)-2-(morpholin-4-yl)propanoyl]amino}-4-(trifluoromethoxy)benzamide, 3-{[2-(morpholin-4-yl)propanoyl]amino}-N-(6-phenylpyridin-3-yl)-4-(trifluoromethoxy)benzamide, N-[6-(2-fluorophenyl)pyridin-3-yl]-3-{[2-methyl-2-(morpholin-4-yl)propanoyl]amino}-4-(trifluoromethoxy)benzamide, 3-[(morpholin-4-ylacetyl)amino}-N-(5-phenyl-1,3-thiazol-2-yl)-4-(trifluoromethoxy)benzamide, 3-[(morpholin-4-ylacetyl)amino]-N-(6-phenylpyridin-3-yl)-4-(trifluoromethoxy)benzamide, N-{3-[(morpholin-4-ylacetyl)amino]-4-(trifluoromethoxy) phenyl}-5-phenyl-1,3-thiazole-2-carboxamide, N-{3-[(morpholin-4-ylacetyl)amino]-4-(trifluoromethoxy)phenyl}-5-phenylthiophene-2-carboxamide, N-{4-tert-butyl-3-[(morpholin-4-ylacetyl)amino]phenyl}-5-phenylthiophene-2-carboxamide, N-{4-methoxy-3-[(morpholin-4-ylacetyl)amino]phenyl}-5-phenyl-1,3-oxazole-2-carboxamide, N-{4-chloro-3-[(morpholin-4-ylacetyl)amino]phenyl}-5-phenylthiophene-2-carboxamide, N-{4-methyl-3-[(morpholin-4-ylacetyl)amino]phenyl}-5-phenylthiophene-2-carboxamide, N-{4-methoxy-3-[(morpholin-4-ylacetyl)amino]phenyl}-5-phenyl-1H-pyrrole-2-carboxamide, N-{4-methoxy-3-[(morpholin-4-ylacetyl)amino]phenyl}-5-phenylthiophene-2-carboxamide, 6-(2,3-difluorophenyl)-N-{3-[(morpholin-4-ylacetyl)amino]-4-(trifluoromethoxy)phenyl}nicotinamide, 6-(3,5-difluorophenyl)-N-{3-[(morpholin-4-ylacetyl)amino]-4-(trifluoromethoxy)phenyl}nicotinamide, 6-(3-fluorophenyl)-N-{3-[(morpholin-4-ylacetyl)amino]-4-(trifluoromethoxy)phenyl}nicotinamide, 6-(2-fluorophenyl)-N-{3-[(morpholin-4-ylacetyl)amino]-4-(trifluoromethoxy)phenyl}nicotinamide, 6-(3-fluorophenyl)-N-{4-methoxy-3-[(morpholin-4-ylacetyl)amino]phenyl}nicotinamide, N-{3-[(morpholin-4-ylacetyl)amino]-4-(trifluoromethoxy)phenyl}-6-phenylnicotinamide, N-{4-methoxy-3-[(morpholin-4-ylacetyl)amino]phenyl}-5-(4-methoxyphenyl)thiophene-2-carboxamide, 4-(difluoromethoxy)-3-({[1-(morpholin-4-yl)cyclopropyl]carbonyl}amino)-N-(5-phenyl-1,3,4-thiadiazol-2-yl)benzamide, 3-({[1-(4-cyclopropylpiperazin-1-yl)cyclopropyl]carbonyl}amino)-4-(difluoromethoxy)-N-(5-phenyl-1,3,4-thiadiazol-2-yl)benzamide, 4-(difluoromethoxy)-3-{[(4-methylpiperazin-1-yl)acetyl]amino}-N-(5-phenyl-1,3,4-thiadiazol-2-yl)benzamide, 3-{[(4-cyclopropylpiperazin-1-yl)acetyl]amino}-4-(difluoromethoxy)-N-(5-phenyl-1,3,4-thiadiazol-2-yl)benzamide, 4-(difluoromethoxy)-3-{[2-(4-methyl piperazin-1-yl)propanoyl]amino}-N-(5-phenyl-1,3,4-thiadiazol-2-yl)benzamide trifluoroacetate (1:1), 4-(methoxymethyl)-3-({[1-(morpholin-4-yl)cyclopropyl]carbonyl}amino)-N-(5-phenyl-1,3,4-thiadiazol-2-yl)benzamide, 3-({[1-(4-cyclopropylpiperazin-1-yl)cyclopropyl]carbonyl}amino)-4-(methoxymethyl)-N-(5-phenyl-1,3,4-thiadiazol-2-yl)benzamide, 4-(methoxymethyl)-3-({[1-(4-methylpiperazin-1-yl)cyclopropyl]carbonyl}amino)-N-(5-phenyl-1,3,4-thiadiazol-2-yl)benzamide hydrochloride (1:1), 4-(methoxymethyl)-3-{[(4-methylpiperazin-1-yl)acetyl]amino}-N-(5-phenyl-1,3,4-thiadiazol-2-yl)benzamide, 4-[(methylsulfonyl)methyl]-3-({[1-(morpholin-4-yl)cyclopropyl]carbonyl}amino)-N-(5-phenyl-1,3,4-thiadiazol-2-yl)benzamide, 3-({[1-(4-cyclopropylpiperazin-1-yl)cyclopropyl]carbonyl}amino)-4-[methylsulfonyl)methyl]-N-(5-, phenyl-1,3,4-thiadiazol-2-yl)benzamide, 3-({[1-(4-methylpiperazin-1-yl)cyclopropyl]carbonyl}amino)-4-[(methylsulfonyl)methyl]-N-(5-phenyl-1,3,4-thiadiazol-2-yl)benzamide, 3-({[1-(morpholin-4-yl)cyclopropyl]carbonyl}amino)-N-(5-phenyl-1,3,4-thiadiazol-2-yl)-4-(trifluoromethoxy)benzamide, 3-({[1-(4-cyclopropylpiperazin-1-yl)cyclopropyl]carbonyl}amino)-N-(5-phenyl-1,3,4-thiadiazol-2-yl)-4-(trifluoromethoxy)benzamide, 3-{[(4-cyclopropylpiperazin-1-yl)acetyl]amino}-N-(5-phenyl-1,3,4-thiadiazol-2-yl)-4-(trifluoromethoxy)benzamide, 3-{[(4-methylpiperazin-1-yl)acetyl]amino}-N-(5-phenyl-1,3,4-thiadiazol-2-yl)-4-(trifluoromethoxy)benzamide, 2-({[1-(morpholin-4-yl)cyclopropyl]carbonyl}amino)-N4-(5-phenyl-1,3,4-thiadiazol-2-10yl)terephthalamide, 3-{[2-(morpholin-4-yl)propanoyl]amino}-N-(5-phenyl-1,3,4-thiadiazol-2-yl)-4-(trifluoromethoxy)benzamide, 3-{[2-(4-methylpiperazin-1-yl)propanoyl]amino}-N-(5-phenyl-1,3,4-thiadiazol-2-yl)-4-(trifluoromethoxy)benzamide, 6-(3,5-difluorophenyl)-N-[3-({[1-(morpholin-4-yl)cyclopropyl]carbonyl}amino)-4-(trifluoromethoxy)phenyl]nicotinamide, N-[3-({[1-(morpholin-4-yl)cyclopropyl]carbonyl}amino)-4-(trifluoromethoxy)phenyl]-6-phenylnicotinamide, 6-(2-fluorophenyl)-N-[4-methoxy-3-({[1-(morpholin-4-yl)cyclopropyl]carbonyl}amino)phenyl]nicotinamide, 3-{[(4-cyclopropylpiperazin-1-yl)acetyl]amino}-N-[6-(2-fluorophenyl)pyridin-3-yl]-4-(trifluoromethoxy)benzamide, N-[6-(2-fluorophenyl)pyridin-3-yl]-3-{[2-(4-methylpiperazin-1-yl)propanoyl]amino}-4-(trifluoromethoxy)benzamide, N-[6-(2-fluorophenyl)pyridin-3-yl]-3-{[(2S)-2-(4-methylpiperazin-1-yl)propanoyl]amino}-4-(trifluoromethoxy)benzamide, N-[6-(2-fluorophenyl)pyridin-3-yl]-3-{[(2R)-2-(4-methylpiperazin-1-yl)propanoyl]amino}-4-(trifluoromethoxy)benzamide, N-[6-(2-fluorophenyl)pyridin-3-yl]-3-{[(2S)-2-(4-methylpiperazin-1-yl)propanoyl]amino}-4-(trifluoromethoxy)benzamide, N-[6-(2-fluorophenyl)pyridin-3-yl]-3-{[(2R)-2-(4-methylpiperazin-1-yl)propanoyl]amino}-4-(trifluoromethoxy)benzamide, 3-({[1-(4-cyclopropylpiperazin-1-yl)cyclopropyl]carbonyl}amino)-N-[6-(2-fluorophenyl)pyridin-3-yl]-4-(trifluoromethoxy)benzamide, N-[6-(2-fluorophenyl)pyridin-3-yl]-3-({[1-(morpholin-4-yl)cyclopropyl]carbonyl}amino)-4-25 (trifluoromethoxy)benzamide, N-[6-(3-fluorophenyl)pyridin-3-yl]-3-{[2-(8-oxa-3-azabicyclo[3.2.1]oct-3-yl)propanoyl]amino}-4-(trifluoromethoxy)benzamide, N-[6-(3,5-difluorophenyl)pyridin-3-yl]-3-({[1-(morpholin-4-yl)cyclopropyl]carbonyl}amino)-4-(trifluoromethoxy)benzamide, N⁴-[6-(2-fluorophenyl)pyridin-3-yl]-2-({[1-(morpholin-4-yl)cyclopropyl]carbonyl}amino)terephthalamide, N-[6-(2-fluorophenyl)pyridin-3-yl]-4-methyl-3-({[1-(morpholin-4-yl)cyclopropyl]carbonyl}amino)benzamide, 4-chloro-N-[6-(2-fluorophenyl)pyridin-3-yl]-3-({[1-(morpholin-4-yl)cyclopropyl]carbonyl}amino)benzamide, N-[6-(2-fluorophenyl)pyridin-3-yl]-4-methoxy-3-({[1-(morpholin-4-yl)cyclopropyl]carbonyl}amino)benzamide, 4-methoxy-3-({[1-(morpholin-4-yl)cyclopropyl]carbonyl}amino)-N-(6-phenylpyridin-3-yl)benzamide, 3-({[1-(morpholin-4-yl)cyclopropyl]carbonyl}amino)-N-(5-phenylpyridin-2-yl)-4-(trifluoromethoxy)benzamide, methyl 2-{[(4-methylpiperazin-1-yl)acetyl]amino}-4-[(5-phenyl-1,3,4-thiadiazol-2-yl)carbamoyl]benzoate, N-[6-(4-aminophenyl)pyridin-3-yl]-3-{[2-(morpholin-4-yl)propanoyl]amino}-4-(trifluoromethoxy)benzamide, N-[6-(4-acetamidophenyl)pyridin-3-yl]-3-{[2-(morpholin-4-yl)propanoyl]amino}-4-(trifluoromethoxy)benzamide, N-{6-[4-(dimethylamino)phenyl]pyridin-3-yl}-3-{[(2S)-2-(morpholin-4-yl)propanoyl]amino}-4-(trifluoromethoxy)benzamide, N-{6-[4-(dimethylamino)phenyl]pyridin-3-yl}-3-{[(2R)-2-(morpholin-4-yl)propanoyl]amino}-4-(trifluoromethoxy)benzamide, N-[6-(4-aminophenyl)pyridin-3-yl]-3-{[(2R)-2-(morpholin-4-yl)propanoyl]amino}-4-(trifluoromethoxy)benzamide, N-[6-(4-aminophenyl)pyridin-3-yl]-3-{[(2S)-2-(morpholin-4-yl)propanoyl]amino}-4-(trifluoromethoxy)benzamide, N-[6-(4-aminophenyl)pyridin-3-yl]-3-{[(2R)-2-(morpholin-4-yl)propanoyl]amino}-4-(trifluoromethoxy)benzamide, N-[6-(4-aminophenyl)pyridin-3-yl]-3-{[(2S)-2-(morpholin-4-yl)propanoyl]amino}-4-(trifluoromethoxy)benzamide, N-{3-[(morpholin-4-ylacetyl)amino]-4-(trifluoromethoxy)phenyl}-5-phenyl-1H-pyrazole-3-carboxamide, N-[6-(4-aminophenyl)pyridin-3-yl]-3-[(morpholin-4-ylacetyl)amino]-4-(trifluoromethoxy)benzamide, 3-[(morpholin-4-ylacetyl)amino]-N-(5-phenylpyridin-2-yl)-4-(trifluoromethoxy)benzamide, 3-{[(4-methylpiperazin-1-yl)acetyl]amino}-N-(5-phenylpyridin-2-yl)-4-(trifluoromethoxy)benzamide, Formiate, 4-(cyclopropyloxy)-N-[6-(2-fluorophenyl)pyridin-3-yl]-3-[(morpholin-4-ylacetyl)amino]benzamide, 4-(cyclopropyloxy)-N-[6-(2-fluorophenyl)pyridin-3-yl]-3-({[1-(morpholin-4-yl)cyclopropyl]carbonyl}amino)benzamide, 4-(cyclopropyloxy)-3-({[1-(4-cyclopropylpiperazin-1-yl)cyclopropyl]carbonyl}amino)-N-[6-(2-fluorophenyl)pyridin-3-yl]benzamide, N-[5-(3-chlorophenyl)-1,3,4-thiadiazol-2-yl]-3-{[(4-methylpiperazin-1-yl)acetyl]amino}-4-(trifluoromethoxy)benzamide, and 4-(cyclopropyloxy)-3-({[1-(morpholin-4-yl)cyclopropyl]carbonyl}amino)-N-(5-phenyl-1,3,4-thiadiazol-2-yl)benzamide

PCT Publication Nos. WO 2014/147182 describes compounds of general formula:

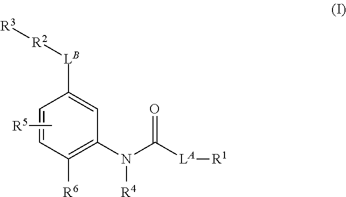

in which
L^{A} represents a methylene or ethylene group, said methylene or ethylene group being optionally substituted, one or more times, identically or differently, with a substituent selected from hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-, 3- to 10-membered heterocycloalkyl-; or, when two substituents are present at the same carbon atom, the two substituents, together with the carbon atom they are attached to, may form a C₃-C₆-cycloalkyl- or 3- to 6-membered heterocycloalkyl-ring; where the ring is optionally substituted one or more times, identically or differently, with a substituent selected from halo-, hydroxy-, cyano-, C₁-C₃-alkyl-, C₁-C₃-alkoxy-;
L^{B} represents —N(H)—C(═O)— or —C(═O)—N(H)—;
R¹ represents a group selected from C₃-C₇-cycloalkyl-, C₄-C₇-cycloalkenyl-, 3- to 10-membered heterocycloalkyl-, 4- to 10-membered heterocycloalkenyl-, aryl-, heteroaryl-, —N(R⁷)—(C₁-C₆-alkyl), —N(R⁷)—C(═O)—O—(C₁-C₆-alkyl), —N(R⁷)R⁷; where the C₃-C₇-cycloalkyl-, C₄-C₇-cycloalkenyl-, 3- to 10-membered heterocycloalkyl-, 4- to 10-membered heterocycloalkenyl-, aryl-, heteroaryl-, and —N(R⁷)—(C₁-C₆-alkyl) group is optionally substituted, one or more times, identically or differently, with a substituent selected from halo-, hydroxy-, cyano-, C₁-C₃-alkyl-, C₁-C₃-alkoxy-, halo-C₁-C₃-alkyl-, hydroxy-C₁-C₃-alkyl-, halo-C₁-C₃-alkoxy-, C₃-C₇-cycloalkyl-, 3-to 10-membered heterocycloalkyl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—, —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰, —S(═O)₂N(H)R⁹, —S(═O)₂NR¹⁰R⁹, —S(═O)(═NR¹⁰)R⁹, —S(═O)(═NR¹⁰)R⁹, —N═S(═O)(R¹⁰)R⁹;
R² represents

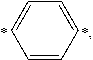

where “*” represents the point of attachment to R³ or L^{B}, respectively; wherein said group is optionally substituted, one or more times, identically or differently, with halo- or a C₁-C₃-alkyl-group;
R³ represents a phenyl group, the phenyl group being optionally substituted, one or more times, identically or differently, with a substituent selected from: halo-, hydroxy-, cyano-, C₁-C₃-alkyl-, C₁-C₃-alkoxy-, halo-C₁-C₃-alkyl-, hydroxy-C₁-C₃-alkyl-, NH₂—C₁-C₃-alkyl-, halo-C₁-C₃-alkoxy-, C₃-C₇-cycloalkyl-, 3- to 10-membered heterocycloalkyl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—, —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰, —S(═O)₂N(H)R⁹, —S(═O)₂NR¹⁰R⁹, —S(═O)(═NR¹⁰)R⁹, —S(═O)(═NR¹⁰)R⁹, —N═S(═O)(R¹⁰)R⁹;
or, when two substituents are present ortho to each other on the phenyl group, the two substituents together form a bridge: *O(CH₂)₂O*, *O(CH₂)O*, *O—C(H)₂C(H)₂*, *NH(C(═O))NH*, where the stars (*) represent the points of attachment to the phenyl group;
R⁴ represents a hydrogen atom or a group selected from C₁-C₆-alkyl-, C₃-C₄-alkenyl-, C₃-C₄-alkynyl-, —(CH₂)ₘ—C₃-C₇-cycloalkyl, —(CH2)ₘ-C₄-C₇-cycloalkenyl, —(CH₂)ₘ-(3- to 10-membered heterocycloalkyl), —(CH₂)ₘ-(4- to 10-membered heterocycloalkenyl), —(CH₂)ₘ-aryl, —(CH₂)ₘ-heteroaryl;
R⁵ represents a hydrogen atom or a halogen atom or a group selected from cyano-, C₁-C₃-alkyl-, C₁-C₃-alkoxy-;
R⁶ represents a group selected from C₁-C₆-alkyl-, C₂-C₆-alkenyl-, C₂-C₆-alkynyl-, C₁-C₆-alkoxy-, C₃-C₆-cycloalkoxy-, halo-, hydroxy-, cyano-, aryl-, heteroaryl-, —N(R⁹)(R¹⁰), —C(═O)—O—R⁹, —C(═O)—N(R⁹)(R¹⁰), R⁹—S—, R⁹—S(═O)—, R⁹—S(═O)₂—; the C₁-C₆-alkyl-, C₂-C₆-alkenyl-, C₂-C₆-alkynyl-, aryl-, heteroaryl- or C₁-C₆-alkoxy group being optionally substituted, one or more times, identically or differently, with halo-, cyano-, nitro-, hydroxy-, C₁-C₃-alkyl-, C₁-C₃-alkoxy-, halo-C₁-C₃-alkoxy-, hydroxy-C₁-C₃-alkoxy-, C₁-C₃-alkoxy-C₁-C₃-alkoxy-, C₃-C₇-cycloalkyl-, C₄-C₇-cycloalkenyl-, 3-to 10-membered heterocycloalkyl-, 4- to 10-membered heterocycloalkenyl-, aryl-, heteroaryl-, —C(═O)R⁹, —C(═O)O—R⁹, —OC(═O)—R⁹, —N(H)C(═O)R⁹, —N(R¹⁰)C(═O)R⁹, —N(H)C(═O)NR¹⁰R⁹, —N(R¹¹)C(═O)NR¹⁰R⁹, —N(H)R⁹, —NR¹⁰R⁹, —C(═O)N(H)R⁹, —C(═O)NR¹⁰R⁹, R⁹—S—, R⁹—S(═O)—, R⁹—S(═O)₂—, —N(H)S(═O)R⁹, —N(R¹⁰)S(═O)R⁹, —S(═O)N(H)R⁹, —S(═O)NR¹⁰R⁹, —N(H)S(═O)₂R⁹, —N(R⁹)S(═O)₂R¹⁰, —S(═O)₂N(H)R⁹, —S(═O)₂NR¹⁰R⁹, —S(═O)(═NR¹⁰)R⁹, —S(═O)(═NR¹⁰)R⁹, —N═S(═O)(R¹⁰)R⁹;
R⁷ represents —H or C₁-C₃-alkyl-;
R⁹, R¹⁰, R¹¹ represent, independently, —H, C₁-C₃-alkyl- or C₃-C₆-cycloalkyl-; the C₁-C₃-alkyl group being optionally substituted with C₁-C₃-alkoxy- or —N(R¹²)R¹³; or
R⁹R¹⁰ together with the atom or the group of atoms they are attached to, form a 3- to 10-membered heterocycloalkyl- or 4- to 10-membered heterocycloalkenyl-group;
R¹², R¹³ represent, independently from each other, —H or C₁-C₃-alkyl-; or together with the atom they are attached to, form a 3- to 10-membered heterocycloalkyl- or 4- to 10-membered heterocycloalkenyl-group;
m represents 0, 1, or 2;
or a tautomer, an N-oxide, a hydrate, a solvate, or a salt thereof, or a mixture of same.

In certain embodiments, the compound described in PCT Publication WO 2014/147182 is described in any of Examples 1-222. In particular embodiments, the compound is one of those listed in Table 3 of WO 2014/147182, i.e., is selected from the group consisting of N-(biphenyl-4-yl)-4-methoxy-3-[(morpholin-4-ylacetyl)amino]benzamide, N-(biphenyl-4-yl)-4-methoxy-3-[(1H-pyrazol-1-ylacetyl)amino]benzamide, N-(biphenyl-4-yl)-3-{[2-methyl-2-(1H-pyrazol-1-yl)propanoyl]amino}-4-(trifluoromethyl)benzamide, N-(biphenyl-4-yl)-4-methoxy-3-{[(1S,4S)-2-oxa-5-azabicyclo[2.2.1]hept-5-ylacetyl]amino}benzamide, N-(biphenyl-4-yl)-4-methoxy-3-[(8-oxa-3-azabicyclo[3.2.1]oct-3-ylacetyl]amino}benzamide, N-(biphenyl-4-yl)-3-[(morpholin-4-ylacetyl)amino]-4-(trifluoromethyl)benzamide, methyl 4-(biphenyl-4-ylcarbamoyl)-2-[(morpholin-4-ylacetyl]amino}benzoate, N-(biphenyl-4-yl)-4-bromo-3-[(morpholin-4-ylacetyl)amino]benzamide, N-(biphenyl-4-yl)-3-{[2-(morpholin-4-yl)propanoyl]amino}-4-(trifluoromethyl)benzamide, N-(biphenyl-4-yl)-3-{[(2S)-2-(morpholin-4-yl)propanoyl]amino}-4-(trifluoromethyl)benzamide, N-(biphenyl-4-yl)-3-{[(2R)-2-(morpholin-4-yl)propanoyl]amino}-4-(trifluoromethyl)benzamide, N-(biphenyl-4-yl)-3-{[2-methyl-2-(morpholin-4-yl)propanoyl]amino}-4-(trifluoromethyl)benzamide, N-(biphenyl-4-yl)-4-cyano-3-[(morpholin-4-ylacetyl)amino]benzamide, N-(biphenyl-4-yl)-4-(2-furyl)-3-[(morpholin-4-ylacetyl)amino]benzamide, N⁴-(biphenyl-4-yl)-N¹-methyl-2-[(morpholin-4-ylacetyl)amino]terephthalamide, N-(biphenyl-4-yl)-3-[(morpholin-4-ylacetyl)amino]-4-(trifluoromethoxy)benzamide, N-(biphenyl-4-yl)-4-isopropoxy-3-[(morpholin-4-ylacetyl)amino]benzamide, N-(biphenyl-4-yl)-4-ethoxy-3-[(morpholin-4-ylacetyl)amino]benzamide, N-{4-methoxy-3-[(1H-pyrazol-1-ylacetyl)amino]phenyl}biphenyl-4-carboxamide, N-(4-methoxy-3-{[2-methyl-2-(morpholin-4-yl)propanoyl]amino}phenyl)biphenyl-4-carboxamide, N-(4-fluoro-3-{[(2S)-2-(morpholin-4-yl)propanoyl]amino}phenyl)biphenyl-4-carboxamide, N-{4-methoxy-3-[(8-oxa-3-azabicyclo[3.2.1]oct-3-ylacetyl)amino]phenyl}biphenyl-4-carboxamide, N-(4-methoxy-3-{[(1S,4S)-2-oxa-5-azabicyclo[2.2.1]hept-5-ylacetyl]amino}phenyl)biphenyl-4-carboxamide, N-(3-{[(4-cyclopropylpiperazin-1-yl)acetyl]amino}-4-methoxyphenyl)biphenyl-4-carboxamide, N-{4-methoxy-3-[(1,4-oxazepan-4-ylacetyl)amino]phenyl}biphenyl-4-carboxamide, N-{4-methoxy-3-[(thiomorpholin-4-ylacetyl)amino]phenyl}biphenyl-4-carboxamide, N-(4-methoxy-3-{[(4-methoxypiperidin-1-yl)acetyl]amino}phenyl)biphenyl-4-carboxamide, N-[3-({[(3S)-3-hydroxypiperidin-1-yl]acetyl}amino)-4-methoxyphenyl]biphenyl-4-carboxamide, N-(3-{[(2,2-dimethylmorpholin-4-yl)acetyl]amino}-4-methoxyphenyl)biphenyl-4-carboxamide, N-[3-({[(3R)-3-hydroxypyrrolidin-1-yl]acetyl}amino)-4-methoxyphenyl]biphenyl-4-carboxamide, N-[4-methoxy-3-({[(3S)-3-methylmorpholin-4-yl]acetyl}amino)phenyl]biphenyl-4-carboxamide, N-(4-methoxy-3-{[2-(morpholin-4-yl)propanoyl]amino}phenyl)biphenyl-4-carboxamide, N-(4-methoxy-3-{[(2S)-2-(8-oxa-3-azabicyclo[3.2.1]oct-3-yl)propanoyl]amino}phenyl)biphenyl-4-carboxamide, N-[3-{[2-(8-oxa-3-azabicyclo[3.2.1]oct-3-yl)propanoyl]amino}-4-(trifluoromethoxy)phenyl]biphenyl-4-carboxamide, N-[3-{[(2S)-2-(8-oxa-3-azabicyclo[3.2.1]oct-3-yl)propanoyl]amino}-4-(trifluoromethoxy)phenyl]biphenyl-4-carboxamide, N-[3-{[2-(morpholin-4-yl)propanoyl]amino}-4-(trifluoromethoxy)phenyl]biphenyl-4-carboxamide, N-[3-{[(2S)-2-(morpholin-4-yl)propanoyl]amino}-4-(trifluoromethoxy)phenyl]biphenyl-4-carboxamide, N-{3-[(morpholin-4-ylacetyl)amino]-4-(trifluoromethoxy)phenyl]biphenyl-4-carboxamide, N-{4-methoxy-3-[(morpholin-4-ylacetyl)amino]phenyl}-3′-methylbiphenyl-4-carboxamide, 3′-cyano-N-{4-methoxy-3-[(morpholin-4-ylacetyl)amino]phenyl}biphenyl-4-carboxamide, 3′-chloro-N-{4-methoxy-3-[(morpholin-4-ylacetyl)amino]phenyl}biphenyl-4-carboxamide, 3′-fluoro-N-{3-[(morpholin-4-ylacetyl)amino]-4-(trifluoromethoxy)phenyl}biphenyl-4-carboxamide, 4′-fluoro-N-{3-[(morpholin-4-ylacetyl)amino]-4-(trifluoromethoxy)phenyl}biphenyl-4-carboxamide, 4′-amino-N-{3-[(morpholin-4-ylacetyl)amino]-4-(trifluoromethoxy)phenyl}biphenyl-4-carboxamide, methyl 4′-({4-methoxy-3-[(morpholin-4-ylacetyl)amino]phenyl}carbamoyl)biphenyl-4-carboxylate, 3′-methoxy-N-{4-methoxy-3-[(morpholin-4-ylacetyl)amino]phenyl}biphenyl-4-carboxamide, 3′-fluoro-N-{4-methoxy-3-[(morpholin-4-ylacetyl)amino]phenyl}biphenyl-4-carboxamide, 2′-fluoro-N-{4-methoxy-3-[(morpholin-4-ylacetyl)amino]phenyl}biphenyl-4-carboxamide, 4′-amino-N-{4-methoxy-3-[(morpholin-4-ylacetyl)amino]phenyl}biphenyl-4-carboxamide, N-{4-ethoxy-3-[(morpholin-4-ylacetyl)amino]phenyl}biphenyl-4-carboxamide, N-(biphenyl-4-yl)-4-[(2-methoxyethoxy)methyl]-3-[(morpholin-4-ylacetyl)amino]benzamide, 4-(benzyloxy)-N-(biphenyl-4-yl)-3-({[1-(morpholin-4-yl)cyclopropyl]carbonyl}amino)benzamide, 4-(3-acetamidopropoxy)-N-(biphenyl-4-yl)-3-({[1-(morpholin-4-yl)cyclopropyl]carbonyl}amino)benzamide, N-(biphenyl-4-yl)-3-({[1-(morpholin-4-yl)cyclopropyl]carbonyl}amino)-4-(trifluoromethoxy)benzamide, N-(biphenyl-4-yl)-4-(methoxymethyl)-3-({[1-(morpholin-4-yl)cyclopropyl]carbonyl}amino)benzamide, N-(biphenyl-4-yl)-4-(methoxymethyl)-3-({[1-(4-methylpiperazin-1-yl)cyclopropyl]carbonyl}amino)benzamide hydrochloride (1:1), N-(biphenyl-4-yl)-4-chloro-3-({[1-(morpholin-4-yl)cyclopropyl]carbonyl}amino)benzamide, N-(biphenyl-4-yl)-3-({[1-(morpholin-4-yl)cyclopropyl]carbonyl}amino)-4-(trifluoromethyl)benzamide, N-(biphenyl-4-yl)-4-methoxy-3-({[1-(4-methylpiperazin-1-yl)cyclopropyl]carbonyl}amino)benzamide hydrochloride (1:1), N-(biphenyl-4-yl)-4-methoxy-3-({[1-(morpholin-4-yl)cyclopropyl]carbonyl}amino)benzamide, N⁴-(biphenyl-4-yl)-N¹-ethyl-2-[(morpholin-4-ylacetyl)amino]terephthalamide, N⁴-(biphenyl-4-yl)-N¹-(2-methoxyethyl)-2-[(morpholin-4-ylacetyl)amino]terephthalamide, N⁴-(biphenyl-4-yl)-N¹-cyclopropyl-2-[(morpholin-4-ylacetyl)amino]terephthalamide, N⁴-(biphenyl-4-yl)-N¹-(3-methoxypropyl)-2-[(morpholin-4-ylacetyl)amino]terephthalamide, N-(biphenyl-4-yl)-4-(methylsulfanyl)-3-[(morpholin-4-ylacetyl)amino]benzamide, N-(biphenyl-4-yl)-4-(cyclopropyloxy)-3-[(morpholin-4-ylacetyl)amino]benzamide, N-(biphenyl-4-yl)-4-(cyclopropyloxy)-3-({[1-(4-methylpiperazin-1-yl)cyclopropyl]carbonyl}amino)benzamide, N-(biphenyl-4-yl)-4-(cyclopropyloxy)-3-({[1-(4-cyclopropylpiperazin-1-yl)cyclopropyl]carbonyl}amino)benzamide, N-(biphenyl-4-yl)-4-[(methylsulfonyl)methyl]-3-[(morpholin-4-ylacetyl)amino]benzamide, and N-(biphenyl-4-yl)-3-[(morpholin-4-ylacetyl)amino]-4-(2,2,2-trifluoroethoxy)benzamide.

In some embodiments, the Wnt pathway inhibitors are polyclonal antibodies. Polyclonal antibodies can be prepared by any known method. In some embodiments, polyclonal antibodies are raised by immunizing an animal (e.g., a rabbit, rat, mouse, goat, donkey) by multiple subcutaneous or intraperitoneal injections of an antigen of interest (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood and/or ascites of the immunized animal. The polyclonal antibodies can be purified from serum or ascites according to standard methods in the art including, but not limited to, affinity chromatography, ion-exchange chromatography, gel electrophoresis, and dialysis.

In some embodiments, the Wnt pathway inhibitors are monoclonal antibodies. Monoclonal antibodies can be prepared using hybridoma methods known to one of skill in the art (see e.g., Kohler and Milstein, 1975, Nature, 256:495-497). In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, FACS, ELISA, and radioimmunoassay). The hybridomas can be propagated either in in vitro culture using standard methods (J. W. Goding, 1996, Monoclonal Antibodies: Principles and Practice, 3rd Edition, Academic Press, San Diego, Calif.) or in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art. The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin proteins. In other embodiments, recombinant monoclonal antibodies, or fragments thereof, can be isolated from phage display libraries.

The polynucleotide(s) encoding a monoclonal antibody can further be modified in a number of different manners using recombinant DNA technology to generate alternative antibodies. In some embodiments, the constant domains of the light and heavy chains of, for example, a mouse monoclonal antibody can be substituted for those regions of, for example, a human antibody to generate a chimeric antibody, or for a non-immunoglobulin polypeptide to generate a fusion antibody. In some embodiments, the constant regions are truncated or removed to generate the desired antibody fragment of a monoclonal antibody. Site-directed or high-density mutagenesis of the variable region can be used to optimize specificity, affinity, etc. of a monoclonal antibody.

In some embodiments, the Wnt pathway inhibitor is a humanized antibody. Typically, humanized antibodies are human immunoglobulins in which amino acid residues of the CDRs are replaced by amino acid residues of a CDR from an immunoglobulin of a non-human species (e.g., mouse, rat, rabbit, hamster, etc.) that have the desired specificity, affinity, and/or binding capability using methods known to one skilled in the art. In some embodiments, Fv framework region amino acid residues of a human immunoglobulin are replaced with corresponding amino acid residues from an antibody of a non-human species that has the desired specificity, affinity, and/or binding capability. In some embodiments, the humanized antibody can be further modified by the substitution of additional amino acid residues either in the Fv framework region and/or within the replaced non-human amino acid residues to refine and optimize antibody specificity, affinity, and/or capability. In general, the humanized antibody will comprise substantially all of at least one, and typically two, variable domain regions containing all, or substantially all, of the CDRs that correspond to the non-human immunoglobulin whereas all, or substantially all, of the framework regions are those of a human immunoglobulin sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. Methods used to generate humanized antibodies are well known in the art.

In certain embodiments, the Wnt pathway inhibitor is a human antibody. Human antibodies can be directly prepared using various techniques known in the art. In some embodiments, immortalized human B lymphocytes immunized in vitro or isolated from an immunized individual that produces an antibody directed against a target antigen can be generated. In some embodiments, the human antibody can be selected from a phage library, where that phage library expresses human antibodies. Alternatively, phage display technology can be used to produce human antibodies and antibody fragments in vitro, from immunoglobulin variable domain gene repertoires from unimmunized donors. Techniques for the generation and use of antibody phage libraries are well-known in the art. Affinity maturation strategies including, but not limited to, chain shuffling (Marks et al., 1992, Bio/Technology, 10:779-783) and site-directed mutagenesis, are known in the art and may be employed to generate high affinity human antibodies.

In some embodiments, human antibodies can be made in transgenic mice that contain human immunoglobulin loci. These mice are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

This invention also encompasses bispecific antibodies that specifically recognize at least one human FZD protein or at least one Wnt protein. Bispecific antibodies are capable of specifically recognizing and binding at least two different epitopes. The different epitopes can either be within the same molecule (e.g., two different epitopes on human FZD5) or on different molecules (e.g., one epitope on FZD5 and a different epitope on a second protein). In some embodiments, the bispecific antibodies are monoclonal human or humanized antibodies. In some embodiments, the antibodies can specifically recognize and bind a first antigen target, (e.g., a FZD protein) as well as a second antigen target, such as an effector molecule on a leukocyte (e.g., CD2, CD3, CD28, CD80, or CD86) or a Fc receptor (e.g., CD64, CD32, or CD16) so as to focus cellular defense mechanisms to the cell expressing the first antigen target. In some embodiments, the antibodies can be used to direct cytotoxic agents to cells which express a particular target antigen. These antibodies possess an antigen-binding arm and an arm which binds a cytotoxic agent or a radionuclide chelator, such as EOTUBE, DPTA, DOTA, or TETA.

Bispecific antibodies can be intact antibodies or antibody fragments. Antibodies with more than two valencies are also contemplated. For example, trispecific antibodies can be prepared (Tutt et al., 1991, J. Immunol., 147:60). Thus, in certain embodiments the antibodies are multispecific. Techniques for making bispecific and multispecific antibodies are known by those skilled in the art.

In certain embodiments, the antibodies (or other polypeptides) described herein may be monospecific. For example, in certain embodiments, each of the one or more antigen-binding sites that an antibody contains is capable of binding (or binds) a homologous epitope on different proteins. In certain embodiments, an antigen-binding site of a monospecific antibody described herein is capable of binding (or binds), for example, FZD5 and FZD7 (i.e., the same epitope is found on both FZD5 and FZD7 proteins).

In certain embodiments, the Wnt pathway inhibitor is an antibody fragment comprising an antigen-binding site. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries to allow rapid and effective identification of monoclonal Fab fragments with the desired specificity for a FZD or Wnt protein or derivatives, fragments, analogs or homologs thereof. In some embodiments, antibody fragments are linear antibody fragments. In certain embodiments, antibody fragments are monospecific or bispecific. In certain embodiments, the Wnt pathway inhibitor is a scFv. Various techniques can be used for the production of single-chain antibodies specific to one or more human FZD proteins or one or more human Wnt proteins.

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis). In some embodiments, an antibody is modified to decrease its serum half-life.

Heteroconjugate antibodies are also within the scope of the present invention. Heteroconjugate antibodies are composed of two covalently joined antibodies. Such antibodies have, for example, been proposed to target immune cells to unwanted cells. It is also contemplated that the heteroconjugate antibodies can be prepared in vitro using known methods in synthetic protein chemistry, including those involving crosslinking agents. For example, immunotoxins can be constructed using a disulfide exchange reaction or by forming a thioether bond. Examples of suitable reagents for this purpose include iminothiolate and methyl-4-mercaptobutyrimidate.

For the purposes of the present invention, it should be appreciated that modified antibodies can comprise any type of variable region that provides for the association of the antibody with the target (i.e., a human FZD protein or a human Wnt protein). In this regard, the variable region may comprise or be derived from any type of mammal that can be induced to mount a humoral response and generate immunoglobulins against the desired tumor-associated antigen. As such, the variable region of the modified antibodies can be, for example, of human, murine, non-human primate (e.g. cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In certain embodiments, the variable domains in both the heavy and light chains are altered by at least partial replacement of one or more CDRs and, if necessary, by partial framework region replacement and sequence modification and/or alteration. Although the CDRs may be derived from an antibody of the same class or even subclass as the antibody from which the framework regions are derived, it is envisaged that the CDRs will be derived preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen-binding site.

Alterations to the variable region notwithstanding, those skilled in the art will appreciate that the modified antibodies of this invention will comprise antibodies (e.g., full-length antibodies or immunoreactive fragments thereof) in which at least a fraction of one or more of the constant region domains has been deleted or otherwise altered so as to provide desired biochemical characteristics such as increased tumor localization and/or increased serum half-life when compared with an antibody of approximately the same immunogenicity comprising a native or unaltered constant region. In some embodiments, the constant region of the modified antibodies will comprise a human constant region. Modifications to the constant region compatible with this invention comprise additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In some embodiments, the modified antibodies will comprise domain deleted constructs or variants wherein the entire CH2 domain has been removed (ΔCH2 constructs). In some embodiments, the omitted constant region domain is replaced by a short amino acid spacer (e.g., 10 amino acid residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete the part of one or more constant region domains that control a specific effector function (e.g. complement C1q binding). Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment sites.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells, release of inflammatory mediators, placental transfer, and control of immunoglobulin production.

In certain embodiments, the Wnt pathway inhibitors are antibodies that provide for altered effector functions. These altered effector functions may affect the biological profile of the administered antibody. For example, in some embodiments, the deletion or inactivation (through point mutations or other means) of a constant region domain may reduce Fc receptor binding of the circulating modified antibody (e.g., anti-FZD antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties. Modifications to the constant region in accordance with this invention may easily be made using well known biochemical or molecular engineering techniques well within the purview of the skilled artisan.

In certain embodiments, a Wnt pathway inhibitor is an antibody does not have one or more effector functions. For instance, in some embodiments, the antibody has no ADCC activity, and/or no CDC activity. In certain embodiments, the antibody does not bind an Fc receptor, and/or complement factors. In certain embodiments, the antibody has no effector function.

The present invention further embraces variants and equivalents which are substantially homologous to the chimeric, humanized, and human antibodies, or antibody fragments thereof, set forth herein. These can contain, for example, conservative substitution mutations, i.e. the substitution of one or more amino acids by similar amino acids. For example, conservative substitution refers to the substitution of an amino acid with another within the same general class such as, for example, one acidic amino acid with another acidic amino acid, one basic amino acid with another basic amino acid or one neutral amino acid by another neutral amino acid. What is intended by a conservative amino acid substitution is well known in the art and described herein.

In certain embodiments, the antibodies described herein are isolated. In certain embodiments, the antibodies described herein are substantially pure.

In some embodiments of the present invention, the Wnt pathway inhibitors are polypeptides. The polypeptides can be recombinant polypeptides, natural polypeptides, or synthetic polypeptides comprising an antibody, or fragment thereof, that bind at least one human FZD protein or at least one Wnt protein. It will be recognized in the art that some amino acid sequences of the invention can be varied without significant effect on the structure or function of the protein. Thus, the invention further includes variations of the polypeptides which show substantial activity or which include regions of an antibody, or fragment thereof, against a human FZD protein or a Wnt protein. In some embodiments, amino acid sequence variations of FZD-binding polypeptides or Wnt-binding polypeptides include deletions, insertions, inversions, repeats, and/or other types of substitutions.

The polypeptides, analogs and variants thereof, can be further modified to contain additional chemical moieties not normally part of the polypeptide. The derivatized moieties can improve the solubility, the biological half-life, and/or absorption of the polypeptide. The moieties can also reduce or eliminate any undesirable side effects of the polypeptides and variants. An overview for chemical moieties can be found in Remington: The Science and Practice of Pharmacy, 22st Edition, 2012, Pharmaceutical Press, London.

The isolated polypeptides that can be used in the methods described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof.

In some embodiments, a DNA sequence encoding a polypeptide of interest may be constructed by chemical synthesis using an oligonucleotide synthesizer. Oligonucleotides can be designed based on the amino acid sequence of the desired polypeptide and selecting those codons that are favored in the host cell in which the recombinant polypeptide of interest will be produced. Standard methods can be applied to synthesize a polynucleotide sequence encoding an isolated polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis, or another method), the polynucleotide sequences encoding a particular polypeptide of interest can be inserted into an expression vector and operatively linked to an expression control sequence appropriate for expression of the protein in a desired host. Proper assembly can be confirmed by nucleotide sequencing, restriction enzyme mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding binding agents (e.g., antibodies or soluble receptors), or fragments thereof, against a human FZD protein or a Wnt protein. For example, recombinant expression vectors can be replicable DNA constructs which have synthetic or cDNA-derived DNA fragments encoding a polypeptide chain of a FZD-binding agent, a Wnt-binding agent, an anti-FZD antibody or fragment thereof, an anti-Wnt antibody or fragment thereof, or a FZD-Fc soluble receptor operatively linked to suitable transcriptional and/or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of an expression control sequence and an expression vector depends upon the choice of host. A wide variety of expression host/vector combinations can be employed. Useful expression vectors for eukaryotic hosts include, for example, vectors comprising expression control sequences from SV40, bovine papilloma virus, adenovirus, and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a FZD-binding or Wnt-binding agent (or a protein to use as an antigen) include prokaryotes, yeast cells, insect cells, or higher eukaryotic cells under the control of appropriate promoters. Prokaryotes include gram-negative or gram-positive organisms, for example E. coli or Bacillus. Higher eukaryotic cells include established cell lines of mammalian origin as described below. Cell-free translation systems may also be employed. Appropriate cloning and expression vectors for use with bacterial, fungal, yeast, and mammalian cellular hosts are well-known in the art. Additional information regarding methods of protein production, including antibody production, can be found, e.g., in U.S. Patent Publication No. 2008/0187954, U.S. Pat. Nos. 6,413,746 and 6,660,501, and International Patent Publication No. WO 2004/009823.

Various mammalian culture systems are used to express recombinant polypeptides. Expression of recombinant proteins in mammalian cells may be preferred because such proteins are generally correctly folded, appropriately modified, and biologically functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived), BHK (hamster kidney fibroblast-derived), HEK-293 (human embryonic kidney-derived) cell lines and variants thereof. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences.

Expression of recombinant proteins in insect cell culture systems (e.g., baculovirus) also offers a robust method for producing correctly folded and biologically functional proteins. Baculovirus systems for production of heterologous proteins in insect cells are well-known to those of skill in the art (see, e.g., Luckow and Summers, 1988, Bio/Technology, 6:47).

Thus, the present invention provides cells comprising the FZD-binding agents or the Wnt-binding agents described herein. In some embodiments, the cells produce the binding agents (e.g., antibodies or soluble receptors) described herein. In certain embodiments, the cells produce an antibody. In certain embodiments, the cells produce antibody OMP-18R5. In some embodiments, the cells produce a soluble receptor. In some embodiments, the cells produce a FZD-Fc soluble receptor. In some embodiments, the cells produce a FZD8-Fc soluble receptor. In some embodiments, the cells produce FZD8-Fc soluble receptor 54F28.

The proteins produced by a transformed host can be purified according to any suitable method. Standard methods include chromatography (e.g., ion exchange, affinity, and sizing column chromatography), centrifugation, differential solubility, or by any other standard technique for protein purification. Affinity tags such as hexa-histidine, maltose binding domain, influenza coat sequence, and glutathione-S-transferase can be attached to the protein to allow easy purification by passage over an appropriate affinity column. Isolated proteins can also be physically characterized using such techniques as proteolysis, mass spectrometry (MS), nuclear magnetic resonance (NMR), high performance liquid chromatography (HPLC), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In some embodiments, an anion exchange resin can be employed, for example, a matrix or substrate having pendant diethylaminoethyl (DEAE) groups. The matrices can be acrylamide, agarose, dextran, cellulose, or other types commonly employed in protein purification. In some embodiments, a cation exchange step can be employed. Suitable cation exchangers include various insoluble matrices comprising sulfopropyl or carboxymethyl groups. In some embodiments, a hydroxyapatite media can be employed, including but not limited to, ceramic hydroxyapatite (CHT). In certain embodiments, one or more reverse-phase HPLC steps employing hydrophobic RP-HPLC media, e.g., silica gel having pendant methyl or other aliphatic groups, can be employed to further purify a binding agent. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange, or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Publication Nos. 2008/0312425, 2008/0177048, and 2009/0187005.

In certain embodiments, the Wnt-binding agent or the FZD-binding agent is a polypeptide that is not an antibody. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a FZD-binding or Wnt-binding polypeptide. In certain embodiments, the polypeptide comprises a protein scaffold of a type selected from the group consisting of protein A, protein G, a lipocalin, a fibronectin domain, an ankyrin consensus repeat domain, and thioredoxin.

In certain embodiments, the binding agents can be used in any one of a number of conjugated (i.e. an immunoconjugate or radioconjugate) or non-conjugated forms. In certain embodiments, antibodies can be used in a non-conjugated form to harness the subject's natural defense mechanisms including complement-dependent cytotoxicity and antibody dependent cellular toxicity to eliminate the malignant or cancer cells.

In some embodiments, the binding agent is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. In some embodiments, conjugates of an antibody and one or more small molecule toxins, such as a calicheamicin, maytansinoids, a trichothene, and CC1065, and the derivatives of these toxins that have toxin activity, can be produced. In certain embodiments, conjugates of an antibody and a cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In certain embodiments, the Wnt pathway inhibitor (e.g., antibody, soluble receptor, or small molecule) is an antagonist of at least one Wnt protein (i.e., 1, 2, 3, 4, 5, 6, 7, 8, 9, or 10 Wnt proteins). In certain embodiments, the Wnt pathway inhibitor inhibits activity of the Wnt protein(s) to which it binds. In certain embodiments, the Wnt pathway inhibitor inhibits at least about 10%, at least about 20%, at least about 30%, at least about 50%, at least about 75%, at least about 90%, or about 100% of the activity of the human Wnt protein(s) to which it binds.

In certain embodiments, the Wnt pathway inhibitor (e.g., antibody, soluble receptor, or small molecule) inhibits binding of at least one human Wnt to an appropriate receptor. In certain embodiments, the Wnt pathway inhibitor inhibits binding of at least one human Wnt protein to one or more human FZD proteins. In some embodiments, the at least one Wnt protein is selected from the group consisting of: Wnt1, Wnt2, Wnt2b/13, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the one or more human FZD proteins are selected from the group consisting of: FZD1, FZD2, FZD3, FZD4, FZD5, FZD6, FZD7, FZD8, FZD9, and FZD10. In certain embodiments, the Wnt pathway inhibitor inhibits binding of one or more Wnt proteins to FZD1, FZD2, FZD4, FZD5, FZD7, and/or FZD8. In certain embodiments, the Wnt pathway inhibitor inhibits binding of one or more Wnt proteins to FZD8. In certain embodiments, the inhibition of binding of a particular Wnt to a FZD protein by a Wnt pathway inhibitor is at least about 10%, at least about 25%, at least about 50%, at least about 75%, at least about 90%, or at least about 95%. In certain embodiments, an agent that inhibits binding of a Wnt to a FZD protein, also inhibits Wnt pathway signaling. In certain embodiments, a Wnt pathway inhibitor that inhibits human Wnt pathway signaling is an antibody. In certain embodiments, a Wnt pathway inhibitor that inhibits human Wnt pathway signaling is a FZD-Fc soluble receptor. In certain embodiments, a Wnt pathway inhibitor that inhibits human Wnt pathway signaling is a FZD8-Fc soluble receptor. In certain embodiments, a Wnt pathway inhibitor that inhibits human Wnt pathway signaling is soluble receptor 54F28.

In certain embodiments, the Wnt pathway inhibitors (e.g., antibody, soluble receptor, or small molecule) described herein are antagonists of at least one human Wnt protein and inhibit Wnt activity. In certain embodiments, the Wnt pathway inhibitor inhibits Wnt activity by at least about 10%, at least about 20%, at least about 30%, at least about 50%, at least about 75%, at least about 90%, or about 100%. In some embodiments, the Wnt pathway inhibitor inhibits activity of one, two, three, four, five or more Wnt proteins. In some embodiments, the Wnt pathway inhibitor inhibits activity of at least one human Wnt protein selected from the group consisting of: Wnt1, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, Wnt10a, Wnt10b, Wnt11, and Wnt16. In some embodiments, the Wnt-binding agent binds at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, the at least one Wnt protein is selected from the group consisting of Wnt1, Wnt2, Wnt2b, Wnt3, Wnt3a, Wnt8a, Wnt8b, Wnt10a, and Wnt10b. In certain embodiments, a Wnt pathway inhibitor that inhibits human Wnt activity is an antibody. In certain embodiments, a Wnt pathway inhibitor that inhibits human Wnt activity is a FZD-Fc soluble receptor. In certain embodiments, a Wnt pathway inhibitor that inhibits human Wnt activity is a FZD8-Fc soluble receptor. In certain embodiments, a Wnt pathway inhibitor that inhibits human Wnt activity is soluble receptor 54F28.

In certain embodiments, the Wnt pathway inhibitor described herein is an antagonist of at least one human FZD protein and inhibits FZD activity. In certain embodiments, the Wnt pathway inhibitor inhibits FZD activity by at least about 10%, at least about 20%, at least about 30%, at least about 50%, at least about 75%, at least about 90%, or about 100%. In some embodiments, the Wnt pathway inhibitor inhibits activity of one, two, three, four, five or more FZD proteins. In some embodiments, the Wnt pathway inhibitor inhibits activity of at least one human FZD protein selected from the group consisting of: FZD1, FZD2, FZD3, FZD4, FZD5, FZD6, FZD7, FZD8, FZD9, and FZD10. In certain embodiments, the Wnt pathway inhibitor inhibits activity of FZD1, FZD2, FZD4, FZD5, FZD7, and/or FZD8. In certain embodiments, the Wnt pathway inhibitor inhibits activity of FZD8. In some embodiments, the Wnt pathway inhibitor is an anti-FZD antibody. In certain embodiments, the Wnt pathway inhibitor is anti-FZD antibody OMP-18R5.

In certain embodiments, the Wnt pathway inhibitor described herein is an antagonist of at least one human Wnt protein and inhibits Wnt signaling In certain embodiments, the Wnt pathway inhibitor inhibits Wnt signaling by at least about 10%, at least about 20%, at least about 30%, at least about 50%, at least about 75%, at least about 90%, or about 100%. In some embodiments, the Wnt pathway inhibitor inhibits signaling by one, two, three, four, five or more Wnt proteins. In some embodiments, the Wnt pathway inhibitor inhibits signaling of at least one Wnt protein selected from the group consisting of Wnt1, Wnt2, Wnt2b, Wnt3, Wnt3a, Wnt7a, Wnt7b, Wnt8a, Wnt8b, Wnt10a, and Wnt10b. In certain embodiments, a Wnt pathway inhibitor that inhibits Wnt signaling is an antibody. In certain embodiments, a Wnt pathway inhibitor that inhibits Wnt signaling is a soluble receptor. In certain embodiments, a Wnt pathway inhibitor that inhibits Wnt signaling is a FZD-Fc soluble receptor. In certain embodiments, a Wnt pathway inhibitor that inhibits Wnt signaling is a FZD8-Fc soluble receptor. In certain embodiments, a Wnt pathway inhibitor that inhibits Wnt signaling is soluble receptor 54F28.

In certain embodiments, a Wnt pathway inhibitor described herein is an antagonist of β-catenin signaling. In certain embodiments, the Wnt pathway inhibitor inhibits β-catenin signaling by at least about 10%, at least about 20%, at least about 30%, at least about 50%, at least about 75%, at least about 90%, or about 100%. In certain embodiments, a Wnt pathway inhibitor that inhibits β-catenin signaling is an antibody. In certain embodiments, a Wnt pathway inhibitor that inhibits β-catenin signaling is an anti-FZD antibody. In certain embodiments, a Wnt pathway inhibitor that inhibits β-catenin signaling is antibody OMP-18R5. In certain embodiments, a Wnt pathway inhibitor that inhibits β-catenin signaling is a soluble receptor. In certain embodiments, a Wnt pathway inhibitor that inhibits β-catenin signaling is a FZD-Fc soluble receptor. In certain embodiments, a Wnt pathway inhibitor that inhibits β-catenin signaling is a FZD8-Fc soluble receptor.

In certain embodiments, the Wnt pathway inhibitor described herein inhibits binding of at least one Wnt protein to a receptor. In certain embodiments, the Wnt pathway inhibitor inhibits binding of at least one human Wnt protein to one or more of its receptors. In some embodiments, the Wnt pathway inhibitor inhibits binding of at least one Wnt protein to at least one FZD protein. In some embodiments, the Wnt-binding agent inhibits binding of at least one Wnt protein to FZD1, FZD2, FZD3, FZD4, FDZ5, FDZ6, FDZ7, FDZ8, FDZ9, and/or FZD10. In certain embodiments, the inhibition of binding of at least one Wnt to at least one FZD protein is at least about 10%, at least about 25%, at least about 50%, at least about 75%, at least about 90%, or at least about 95%. In certain embodiments, a Wnt pathway inhibitor that inhibits binding of at least one Wnt to at least one FZD protein further inhibits Wnt pathway signaling and/or β-catenin signaling. In certain embodiments, a Wnt pathway inhibitor that inhibits binding of at least one human Wnt to at least one FZD protein is an antibody. In certain embodiments, a Wnt pathway inhibitor that inhibits binding of at least one human Wnt to at least one FZD protein is an anti-FZD antibody. In certain embodiments, a Wnt pathway inhibitor that inhibits binding of at least one human Wnt to at least one FZD protein is antibody OMP-18R5. In certain embodiments, a Wnt pathway inhibitor that inhibits binding of at least one human Wnt to at least one FZD protein is a soluble receptor. In certain embodiments, a Wnt pathway inhibitor that inhibits binding of at least one human Wnt to at least one FZD protein is a FZD-Fc soluble receptor. In certain embodiments, a Wnt pathway inhibitor that inhibits binding of at least one human Wnt to at least one FZD protein is a FZD8-Fc soluble receptor. In certain embodiments, a Wnt pathway inhibitor that inhibits binding of at least one human Wnt to at least one FZD protein is FZD8-Fc soluble receptor 54F28.

In certain embodiments, the Wnt pathway inhibitor described herein blocks binding of at least one Wnt to a receptor. In certain embodiments, the Wnt pathway inhibitor blocks binding of at least one human Wnt protein to one or more of its receptors. In some embodiments, the Wnt pathway inhibitor blocks binding of at least one Wnt to at least one FZD protein. In some embodiments, the Wnt pathway inhibitor blocks binding of at least one Wnt protein to FZD1, FZD2, FZD3, FZD4, FDZ5, FDZ6, FDZ7, FDZ8, FDZ9, and/or FZD10. In certain embodiments, the blocking of binding of at least one Wnt to at least one FZD protein is at least about 10%, at least about 25%, at least about 50%, at least about 75%, at least about 90%, or at least about 95%. In certain embodiments, a Wnt pathway inhibitor that blocks binding of at least one Wnt protein to at least one FZD protein further inhibits Wnt pathway signaling and/or β-catenin signaling. In certain embodiments, a Wnt pathway inhibitor that blocks binding of at least one human Wnt to at least one FZD protein is an antibody. In certain embodiments, a Wnt pathway inhibitor that blocks binding of at least one human Wnt to at least one FZD protein is an anti-FZD antibody. In certain embodiments, a Wnt pathway inhibitor that blocks binding of at least one human Wnt to at least one FZD protein is antibody OMP-18R5. In certain embodiments, a Wnt pathway inhibitor that blocks binding of at least one human Wnt to at least one FZD protein is a soluble receptor. In certain embodiments, a Wnt pathway inhibitor that blocks binding of at least one human Wnt to at least one FZD protein is a FZD-Fc soluble receptor. In certain embodiments, a Wnt pathway inhibitor that blocks binding of at least one human Wnt to at least one FZD protein is a FZD8-Fc soluble receptor. In certain embodiments, a Wnt pathway inhibitor that blocks binding of at least one human Wnt to at least one FZD protein is soluble receptor 54F28.

In certain embodiments, the Wnt pathway inhibitor described herein inhibits Wnt pathway signaling. It is understood that a Wnt pathway inhibitor that inhibits Wnt pathway signaling may, in certain embodiments, inhibit signaling by one or more receptors in the Wnt signaling pathway but not necessarily inhibit signaling by all receptors. In certain alternative embodiments, Wnt pathway signaling by all human receptors may be inhibited. In certain embodiments, Wnt pathway signaling by one or more receptors selected from the group consisting of FZD1, FZD2, FZD3, FZD4, FDZS, FDZ6, FDZ7, FDZ8, FDZ9, and FZD10 is inhibited. In certain embodiments, the inhibition of Wnt pathway signaling by a Wnt pathway inhibitor is a reduction in the level of Wnt pathway signaling of at least about 10%, at least about 25%, at least about 50%, at least about 75%, at least about 90%, or at least about 95%. In some embodiments, a Wnt pathway inhibitor that inhibits Wnt pathway signaling is an antibody. In some embodiments, a Wnt pathway inhibitor that inhibits Wnt pathway signaling is an anti-FZD antibody. In some embodiments, a Wnt pathway inhibitor that inhibits Wnt pathway signaling is antibody OMP-18R5. In some embodiments, a Wnt pathway inhibitor that inhibits Wnt pathway signaling is a soluble receptor. In some embodiments, a Wnt pathway inhibitor that inhibits Wnt pathway signaling is a FZD-Fc soluble receptor. In some embodiments, a Wnt pathway inhibitor that inhibits Wnt pathway signaling is a FZD8-Fc soluble receptor. In some embodiments, a Wnt pathway inhibitor that inhibits Wnt pathway signaling is soluble receptor 54F28.

In certain embodiments, the Wnt pathway inhibitor described herein inhibits activation of β-catenin. It is understood that a Wnt pathway inhibitor that inhibits activation of β-catenin may, in certain embodiments, inhibit activation of β-catenin by one or more receptors, but not necessarily inhibit activation of β-catenin by all receptors. In certain alternative embodiments, activation of β-catenin by all human receptors may be inhibited. In certain embodiments, activation of β-catenin by one or more receptors selected from the group consisting of FZD1, FZD2, FZD3, FZD4, FDZS, FDZ6, FDZ7, FDZ8, FDZ9, and FZD10 is inhibited. In certain embodiments, the inhibition of activation of β-catenin by a Wnt-binding agent is a reduction in the level of activation of β-catenin of at least about 10%, at least about 25%, at least about 50%, at least about 75%, at least about 90%, or at least about 95%. In some embodiments, a Wnt pathway inhibitor that inhibits activation of β-catenin is an antibody. In some embodiments, a Wnt pathway inhibitor that inhibits activation of β-catenin is an anti-FZD antibody. In some embodiments, a Wnt pathway inhibitor that inhibits activation of β-catenin is antibody OMP-18R5. In some embodiments, a Wnt pathway inhibitor that inhibits activation of β-catenin is a soluble receptor. In some embodiments, a Wnt pathway inhibitor that inhibits activation of β-catenin is a FZD-Fc soluble receptor. In some embodiments, a Wnt pathway inhibitor that inhibits activation of β-catenin is a FZD8-Fc soluble receptor. In some embodiments, a Wnt pathway inhibitor that inhibits activation of β-catenin is soluble receptor 54F28.

In vivo and in vitro assays for determining whether a Wnt pathway inhibitor inhibits β-catenin signaling are known in the art. For example, cell-based, luciferase reporter assays utilizing a TCF/Luc reporter vector containing multiple copies of the TCF-binding domain upstream of a firefly luciferase reporter gene may be used to measure β-catenin signaling levels in vitro (Gazit et al., 1999, Oncogene, 18; 5959-66; TOPflash, Millipore, Billerica Mass.). The level of β-catenin signaling in the presence of one or more Wnt proteins (e.g., Wnt(s) expressed by transfected cells or provided by Wnt-conditioned media) in the presence of a binding agent is compared to the level of signaling without the binding agent present. In addition to the TCF/Luc reporter assay, the effect of a binding agent (or candidate agent) on β-catenin signaling may be measured in vitro or in vivo by measuring the effect of the agent on the level of expression of β-catenin-regulated genes, such as c-myc (He et al., 1998, Science, 281:1509-12), cyclin D1 (Tetsu et al., 1999, Nature, 398:422-6), and/or fibronectin (Gradl et al. 1999, Mol. Cell Biol., 19:5576-87). In certain embodiments, the effect of a binding agent on β-catenin signaling may also be assessed by measuring the effect of the agent on the phosphorylation state of Dishevelled-1, Dishevelled-2, Dishevelled-3, LRP5, LRP6, and/or β-catenin.

In certain embodiments, a Wnt pathway inhibitor has one or more of the following effects: inhibit proliferation of tumor cells, inhibit tumor growth, reduce the frequency of cancer stem cells in a tumor, reduce the tumorigenicity of a tumor, reduce the tumorigenicity of a tumor by reducing the frequency of cancer stem cells in the tumor, trigger cell death of tumor cells, induce cells in a tumor to differentiate, differentiate tumorigenic cells to a non-tumorigenic state, induce expression of differentiation markers in the tumor cells, prevent metastasis of tumor cells, or decrease survival of tumor cells.

In certain embodiments, a Wnt pathway inhibitor is capable of inhibiting tumor growth. In certain embodiments, a Wnt pathway inhibitor is capable of inhibiting tumor growth in vivo (e.g., in a xenograft mouse model, and/or in a human having cancer). In some embodiments, the tumor is a tumor selected from the group consisting of colorectal tumor, colon tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is melanoma. In certain embodiments, the tumor is a colorectal tumor. In certain embodiments, the tumor is a pancreatic tumor. In certain embodiments, the tumor is a breast tumor. In certain embodiments, the tumor is a Wnt-dependent tumor.

In certain embodiments, a Wnt pathway inhibitor is capable of reducing the tumorigenicity of a tumor. In certain embodiments, a Wnt pathway inhibitor is capable of reducing the tumorigenicity of a tumor comprising cancer stem cells in an animal model, such as a mouse xenograft model. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the number or frequency of cancer stem cells is determined by limiting dilution assay using an animal model. Additional examples and guidance regarding the use of limiting dilution assays to determine a reduction in the number or frequency of cancer stem cells in a tumor can be found, e.g., in International Publication No. WO 2008/042236, and U.S. Patent Publication Nos. 2008/0064049 and 2008/0178305.

In certain embodiments, the Wnt pathway inhibitors described herein are active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that is active in vivo for at least 1 hour, at least about 2 hours, at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that is active in vivo for at least 1 hour, at least about 2 hours, at least about 5 hours, at least about 10 hours, at least about 24 hours, at least about 2 days, at least about 3 days, at least about 1 week, or at least about 2 weeks.

In certain embodiments, the Wnt pathway inhibitors described herein have a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is an IgG (e.g., IgG1 or IgG2) antibody that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. In certain embodiments, the Wnt pathway inhibitor is a fusion protein that has a circulating half-life in mice, cynomolgus monkeys, or humans of at least about 5 hours, at least about 10 hours, at least about 24 hours, at least about 2 days, at least about 3 days, at least about 1 week, or at least about 2 weeks. Methods of increasing (or decreasing) the half-life of agents such as polypeptides and antibodies are known in the art. For example, known methods of increasing the circulating half-life of IgG antibodies include the introduction of mutations in the Fc region which increase the pH-dependent binding of the antibody to the neonatal Fc receptor (FcRn) at pH 6.0 (see, e.g., U.S. Patent Publication Nos. 2005/0276799, 2007/0148164, and 2007/0122403). Known methods of increasing the circulating half-life of antibody fragments lacking the Fc region include such techniques as PEGylation.

### IV. Immunotherapeutic Agents

The present invention provides Wnt pathway inhibitors for use in combination therapy with immunotherapeutic agents for modulating immune responses, inhibiting tumor growth, and/or for the treatment of cancer. In some embodiments of the methods described herein, a immunotherapeutic agent is selected from the group consisting of: a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

In some embodiments, an immunotherapeutic agent is selected from the group consisting of: a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, and/or an IDO1 antagonist.

In some embodiments, the PD-1 antagonist is an antibody that specifically binds PD-1. In some embodiments, the antibody that binds PD-1 is Merck (KEYTRUDA, MK-3475; Merck), pidilizumab (CT-011; Curetech Ltd.), nivolumab (OPDIVO, BMS-936558, MDX-1106; Bristol Myer Squibb), MEDI0680 (AMP-514; AstraZenenca/MedImmune), REGN2810 (Regeneron Pharmaceuticals), BGB-A317 (BeiGene Ltd.), PDR-001 (Novartis), or STI-A1110 (Sorrento Therapeutics). In some embodiments, the antibody that binds PD-1 is described in PCT Publication WO 2014/179664, for example, an antibody identified as APE2058, APE1922, APE1923, APE1924, APE 1950, or APE1963 (Anaptysbio), or an antibody containing the CDR regions of any of these antibodies. In other embodiments, the PD-1 antagonist is a fusion protein that includes PD-L2, for example, AMP-224 (AstraZeneca/MedImmune). In other embodiments, the PD-1 antagonist is a peptide inhibitor, for example, AUNP-12 (Aurigene).

In some embodiments, the PD-L1 antagonist is an antibody that specifically binds PD-L1. In some embodiments, the antibody that binds PD-L1 is atezolizumab (RG7446, MPDL3280A; Genentech), MEDI4736 (AstraZeneca/MedImmune), BMS-936559 (MDX-1105; Bristol Myers Squibb), avelumab (MSB0010718C; Merck KGaA), KD033 (Kadmon), the antibody portion of KD033, or STI-A1014 (Sorrento Therapeutics). In some embodiments, the antibody that binds PD-L1 is described in PCT Publication WO 2014/055897, for example, Ab-14, Ab-16, Ab-30, Ab-31, Ab-42, Ab-50, Ab-52, or Ab-55, or an antibody that contains the CDR regions of any of these antibodies.

In some embodiments, the CTLA-4 antagonist is an antibody that specifically binds CTLA-4. In some embodiments, the antibody that binds CTLA-4 is ipilimumab (YERVOY) or tremelimumab (CP-675,206). In some embodiments, the CTLA-4 antagonist a CTLA-4 fusion protein, for example, KAHR-102 (Kahr Medical Ltd.).

In some embodiments, the LAG3 antagonist is an antibody that specifically binds LAG3. In some embodiments, the antibody that binds LAG3 is IMP701 (Prima BioMed), IMP731 (Prima BioMed/GlaxoSmithKline), BMS-986016 (Bristol Myer Squibb), LAG525 (Novartis), and GSK2831781 (GlaxoSmithKline). In some embodiments, the LAG3 antagonist includes a soluble LAG3 receptor, for example, IMP321 (Prima BioMed).

In some embodiments, the KIR antagonist is an antibody that specifically binds MR. In some embodiments, the antibody that binds KIR is lirilumab.

In some embodiments, an immunotherapeutic agent is selected from the group consisting of: a CD28 agonist, a 4-1BB agonist, an OX40 agonist, a CD27 agonist, a CD80 agonist, a CD86 agonist, a CD40 agonist, and a GITR agonist.

In some embodiments, the OX40 agonist includes OX40 ligand, or an OX40-binding portion thereof. For example, the OX40 agonist may be MEDI6383 (AstraZeneca). In some embodiments, the OX40 agonist is an antibody that specifically binds OX40. In some embodiments, the antibody that binds OX40 is MEDI6469 (AstraZeneca/MedImmune), MEDI0562 (AstraZeneca/MedImmune), or MOXR0916 (RG7888; Genentech). In some embodiments, the OX40 agonist is a vector (e.g., an expression vector or virus, such as an adenovirus) capable of expressing OX40 ligand. In some embodiments the OX40-expressing vector is Delta-24-RGDOX (DNAtrix) or DNX2401 (DNAtrix).

In some embodiments, the 4-1BB (CD137) agonist is a binding molecule, such as an anticalin. In some embodiments, the anticalin is PRS-343 (Pieris AG). In some embodiments, the 4-1BB agonist is an antibody that specifically binds 4-1BB. In some embodiments, antibody that binds 4-1BB is PF-2566 (PF-05082566; Pfizer) or urelumab (BMS-663513; Bristol Myer Squibb).

In some embodiments, the CD27 agonist is an antibody that specifically binds CD27. In some embodiments, the antibody that binds CD27 is varlilumab (CDX-1127; Celldex).

In some embodiments, the GITR agonist comprises GITR ligand or a GITR-binding portion thereof. In some embodiments, the GITR agonist is an antibody that specifically binds GITR. In some embodiments, the antibody that binds GITR is TRX518 (GITR, Inc.), MK-4166 (Merck), or INBRX-110 (Five Prime Therapeutics/Inhibrx).

In some embodiments, immunotherapeutic agents include, but are not limited to, cytokines such as chemokines, interferons, interleukins, lymphokines, and members of the tumor necrosis factor (TNF) family. In some embodiments, immunotherapeutic agents include immunostimulatory oligonucleotides, such as CpG dinucleotides.

In some embodiments, a immunotherapeutic agent includes, but is not limited to, anti-PD-1 antibodies, anti-PD-L1 antibodies, anti-PD-L2 antibodies, anti-CTLA-4 antibodies, anti-CD28 antibodies, anti-CD80 antibodies, anti-CD86 antibodies, anti-4-1BB antibodies, anti-OX40 antibodies, anti-KIR antibodies, anti-Tim-3 antibodies, anti-LAG3 antibodies, anti-CD27 antibodies, anti-CD40 antibodies, anti-GITR antibodies, anti-TIGIT antibodies, anti-CD20 antibodies, anti-CD96 antibodies, or anti-IDO1 antibodies.

### EXAMPLES

### Example 1

### Effect of Wnt Inhibitors on Tumor Growth Alone and in Combination With an Immune Checkpoint Inhibitor

Vantictumab (18R5) or ipafricept (54F28) in combination with an anti-CTLA-4 antibody significantly reduce B16F10 melanoma growth. B16F10 melanoma cells were implanted into the rear flanks of C57b16/J mice. When tumors reached a mean tumor volume of ˜100 mm³ as measured by electronic caliper, mice were grouped and treated with a control antibody (Hamster IgG, 10 mg/kg, 3QW), 18R5 (45 mg/kg, Q2W), anti-CTLA-4 (4F10-11, 10 mg/kg, 3QW), 54F28 (50 mg/kg, Q2W), or a combination of 18R5+ anti-CTLA-4 or 54F28+ anti-CTLA-4. The results show significant anti-tumor growth with single agent 54F28 (p<0.002) and when anti-CTLA-4 is combined with 54F28 (p<0.0002) or 18R5 (p<0.001) vs control. Data shown represents the mean ±SEM (n=10).

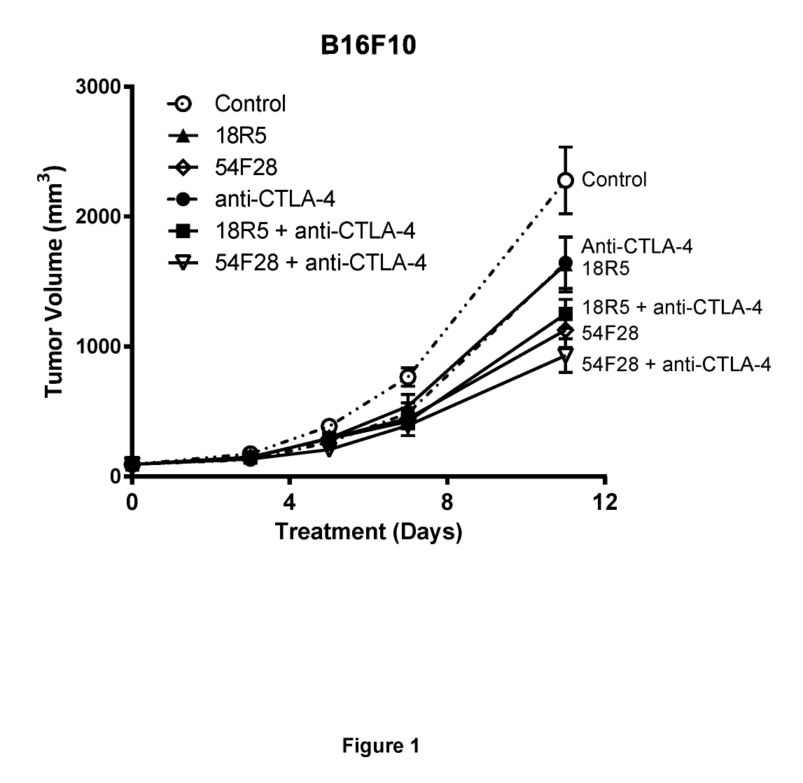

As shown in FIG. 1, the combination of 18R5 and the anti-CTLA-4 antibody produced a greater reduction in tumor cell growth than either 18R5 or the anti-CTLA-4 antibody alone. Similarly, the combination of 54F28 and the anti-CTLA-4 antibody produced a greater reduction in tumor cell growth than either 54F28 or the anti-CTLA-4 antibody alone.

### Example 2

### Effect of Wnt Inhibitors Alone and in Combination With an Immune Checkpoint Inhibitor on T Cell Activation

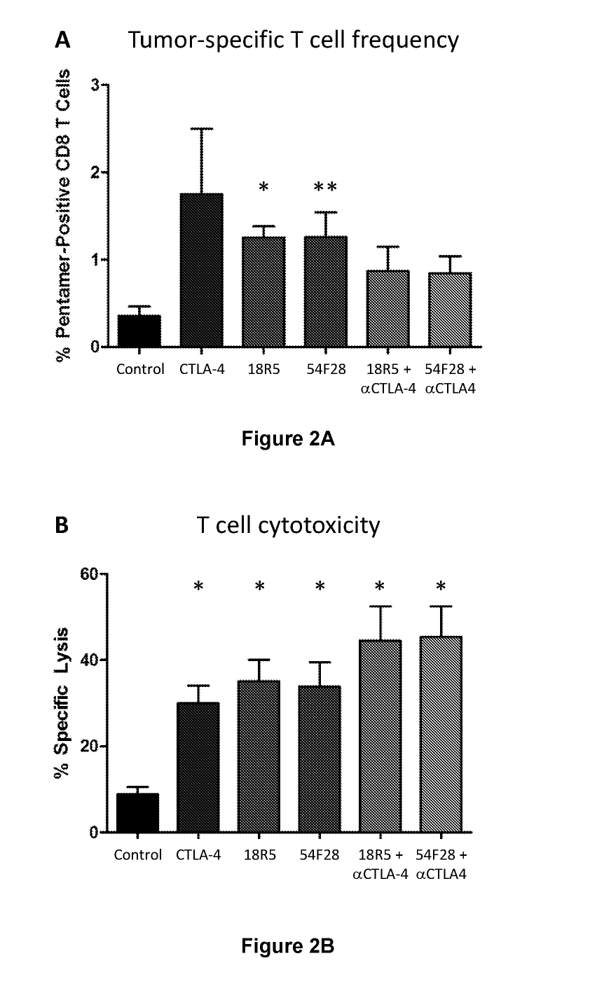

Splenocytic T cells were isolated from the B16F10 tumor-bearing mice described in Example 1. As shown in FIG. 2A, the both the anti-CTLA-4 antibody and the Wnt inhibitors increased the frequency of tumor-specific T cells. Here, total splenocytes were isolated from B16F10 tumor-bearing mice treated as described in Example 1. The frequency of mgp100-specific CD8 T cells was analyzed by pentamer staining of total splenocytes using the ProS Pentamer for mgp100 (Prolmmune, Inc., Sarasota, Fla.), which contains the peptide sequence KVPRNQDWL presented within a pentamer of H-2D^{b} (the MHC class I allele expressed by C56BL/6 mice). Because CD8 antibodies can interfere with the binding of the pentamer, CD8 T cells were identified indirectly as CD3-positive cells that were negative for expression of CD4 and the pan-NK cell marker NK1.1. Results are shown as the percentage of CD8 T cells that were labeled by the pentamer. *,p=0.003 vs. Hamster IgG; **,p=0.059 vs. Hamster IgG.

To measure changes in T cell toxicity, the mouse melanoma cell line B16F10 was cultured in DMEM culture medium supplemented with 10% (v/v) fetal bovine serum (FBS), 2 mM L-glutamine, and 100 U/mL penicillin at 37° C. in a humidified atmosphere of 5% CO₂. Total splenocytes from were cultured for 6 days in RPMI 1640 medium supplemented with 10% FBS, 2 mM L-glutamine, 100 U/mL penicillin, and 1 μg/mL mgp100 peptide (Anaspec). B16F10 target cells were labeled with 10 μM calcein AM (Life Technologies) for 1 hour at 37° C. and then combined with the splenocytes at an effector:target ratio of 25:1. Following a four-hour incubation at 37° C., cell-free supernatants were harvested and calcein release was quantified on a fluorometer at an excitation of 485 nm and an emission of 535 nm. The percentage of specific cell lysis was determined as: % lysis=100×(ER-SR)/(MR-SR), where ER, SR, and MR represent experimental, spontaneous, and maximum calcein release, respectively. Spontaneous release is the fluorescence emitted by target cells incubated in media alone (i.e., in the absence of effector cells), while maximum release is determined by lysing target cells with an equal volume of 10% SDS. For all groups, p<0.05 vs. Hamster IgG.

As shown in FIG. 2B, T cell cytoxocity was increased by anti-CTLA4, 18R5, and 54F28 alone. The increase in T cell cytotoxicity was even greater for the 18R5+ anti-CTLA-4 and 54F28+ anti-CTLA4 combinations.

### Example 3

### Effect of Adding Wnt Inhibitors on Immune Checkpoint Inhibitor Therapy

The effect of the anti-Fzd antibody, OMP-18R5, on melanoma cell line tumor growth was tested animals receiving the combination of anti-CTLA-4 and anti-PD-L1 antibodies. In these experiments, ten thousand B16F1 melanoma cells were injected subcutaneously into C57BL6J mice. On days 4, 7, and 10 post-implantation, “GVAX” tumor vaccine was administered by the injection of 2 million mitomycin C-treated cells of a B16F1 subclone stably transfected with a plasmid encoding m-GM-CSF (with GM-CSF expression confirmed by ELISA), as previously described by Curran and Allison, Cancer Res 69:7747, 2009. Anti-CTLA-4 clone 9D9 (BioXCell; West Lebanon, N.H.) was dosed on days 5, 8, and 12. Anti-PD-L1 clone 10F.9G2 (BioXCell) was dosed on days 5, 8, 12, 14, 19, 22, and 26, and either 1B7.11 isotype or murine chimera 18R5 were administered days 5, 12, 19, and 26 following parental B16F1 cell implantation.

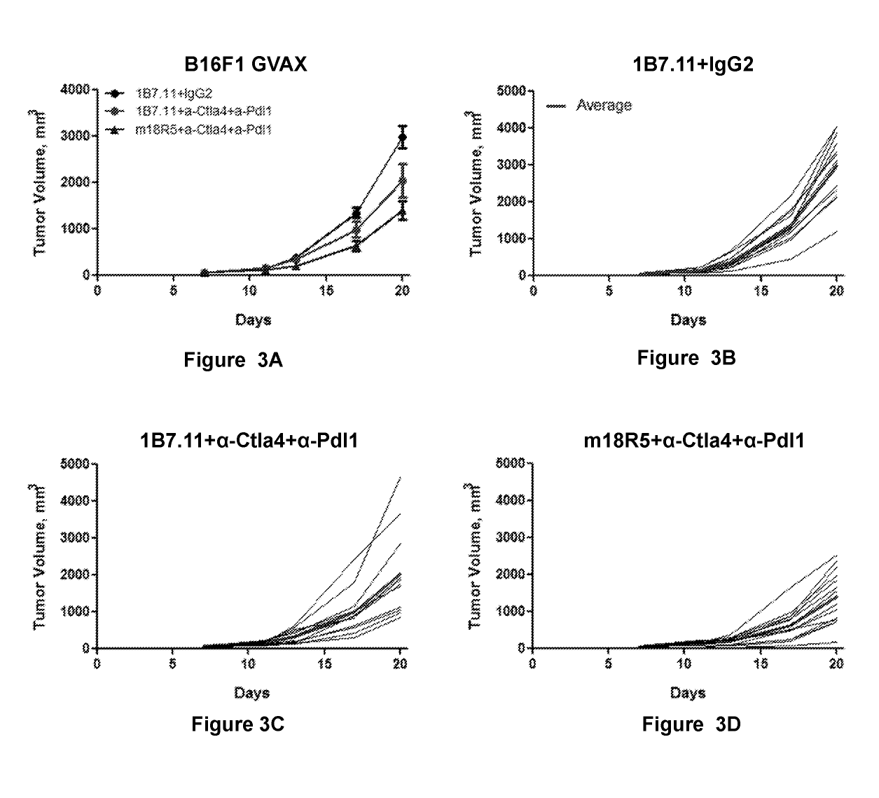

As shown in FIGS. 3A-3D, the addition of 18R5 decreased tumor growth as compared to administration of the anti-CTLA-4 and anti-PD-L1 antibodies without 18R5.

### Example 4

### Effect of Adding Wnt Inhibitors on Cytokine Secretion During Immune Checkpoint Inhibitor Therapy

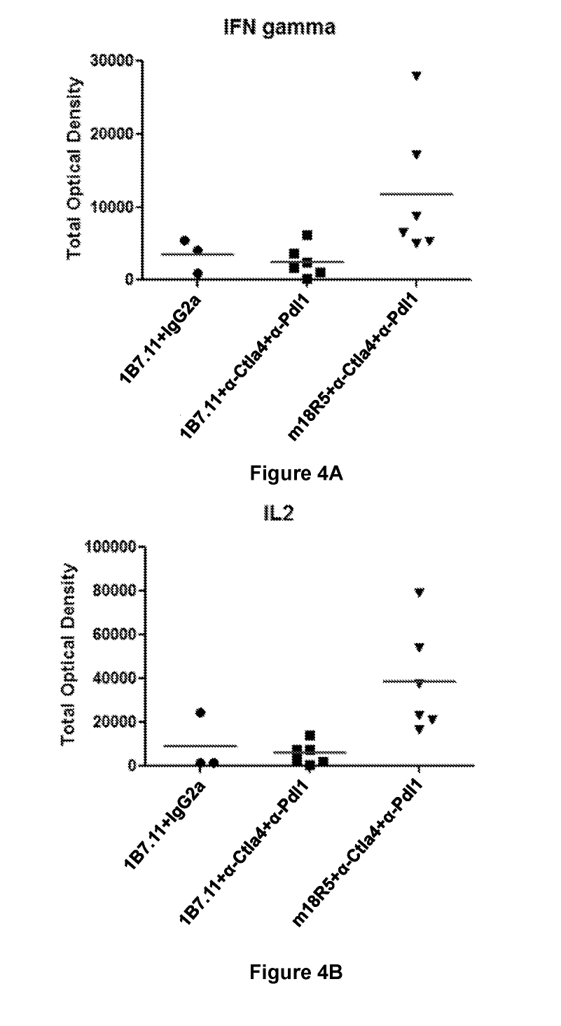

Using the ELISpot cytokine secretion assay, secretion of IFN-gamma and IL-2 was measured following 18R5 treatment. In these assays, splenocytes were harvested from a minimum of four mice per group, filtered, and the red blood cells lysed and re-suspended in RPMI+10% FBS+penn/strep at a concentration of 10⁶ cells/ml in the presence of 1 μg/ml hgp100 peptide. Cells were plated on pre-coated ELISpot plates, incubated overnight and processed according to manufacturer's instructions (MabTech, Cincinnati, Ohio). Developed plates were read on a BioSys BioReader 6000-F b. The results from this assay are shown in FIG. 4A (IFN-gamma) and FIG. 4B (IL-2). Values shown are total optical density. As shown in these figures, IFN-gamma and IL-2 were increased by addition of 18R5 therapy.

### Example 5

### Effect of Adding Wnt Inhibitors on T-Cell Cytotoxicity During Immune Checkpoint Inhibitor Therapy

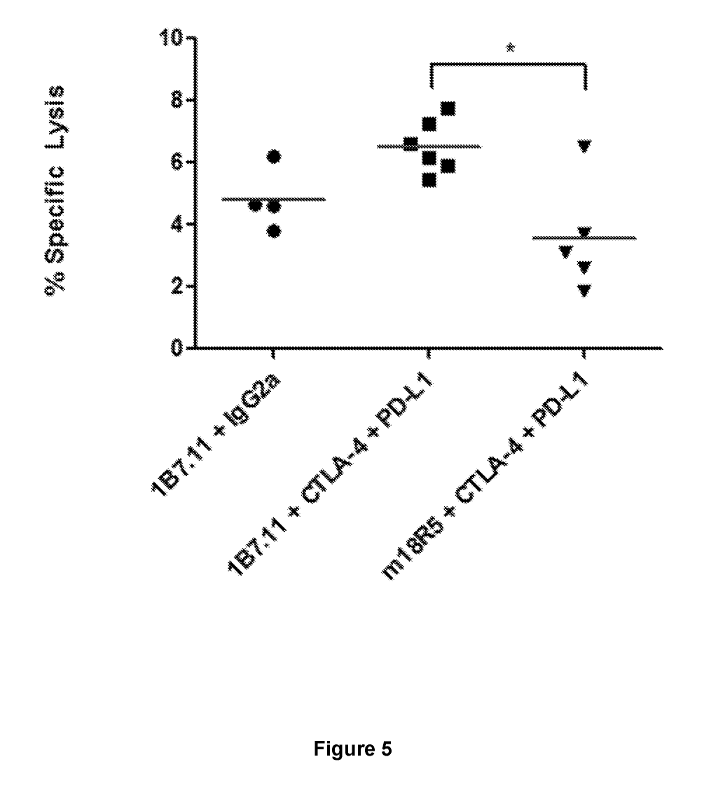

A T-cell cytotoxicity assay was also performed. Briefly, splenocytes were harvested from a minimum of four mice per group, filtered, and the red blood cells lysed and re-suspended in RPMI+10% FBS+penn/strep at a concentration of 2×10⁶ cells/ml. Splenocytes were incubated with 1 μg/ml hgp100 peptide for nine days. On day 9 after plating splenocytes, B16F1 cells in culture were incubated with 10 uM calcein AM viability dye per 10⁶ cells at 37° C. for one hour. Cells were then incubated with 1 μg/ml hgp100 for an additional hour at 37° C. before being washed to remove excess dye. Splenocytes were re-suspended at 2.5×10⁶ cells/ml, B16F1 cells were re-suspended at 10⁵ cells/ml and 100 μl of each combined in each well of a V-bottom plate for an effector:target ratio of 25:1, with splenocytes from each individual mouse plated in triplicate. Labeled B16F1 cells were incubated in 5% SDS to determine maximum lysis conditions, while labeled cells were incubated in medium alone to determine minimum lysis conditions. The plate was then centrifuged at 1000 rpm for four minutes and then incubated at 37° C. for four hours. Culture supernatants were then harvested and fluorescence of released calcein AM was read at 485 nm. Percent specific lysis was determined by dividing the value of the experimental sample by the difference in values between maximum and minimum lysis conditions. The results from these experiments, which are shown in FIG. 5, did not show an increase in T cell cytoxocity from the combination of the Wnt inhibitor with anti-CTLA-4+ anti-PD-L1 as compared to controls.

### Example 6

### Effect of Adding Wnt Inhibitors on T-Cell Tumor Infiltration During Immune Checkpoint Inhibitor Therapy

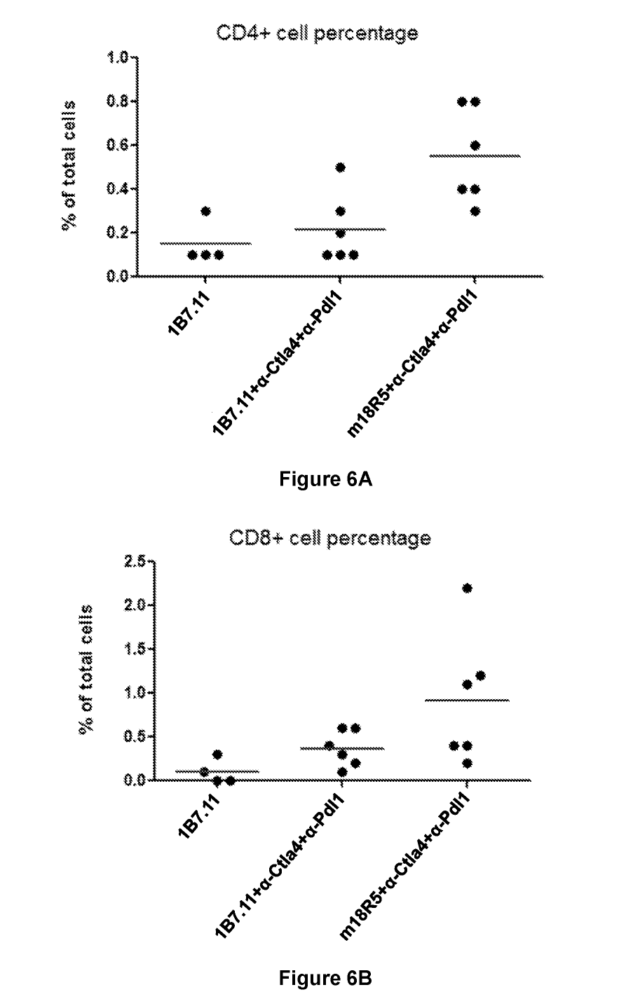

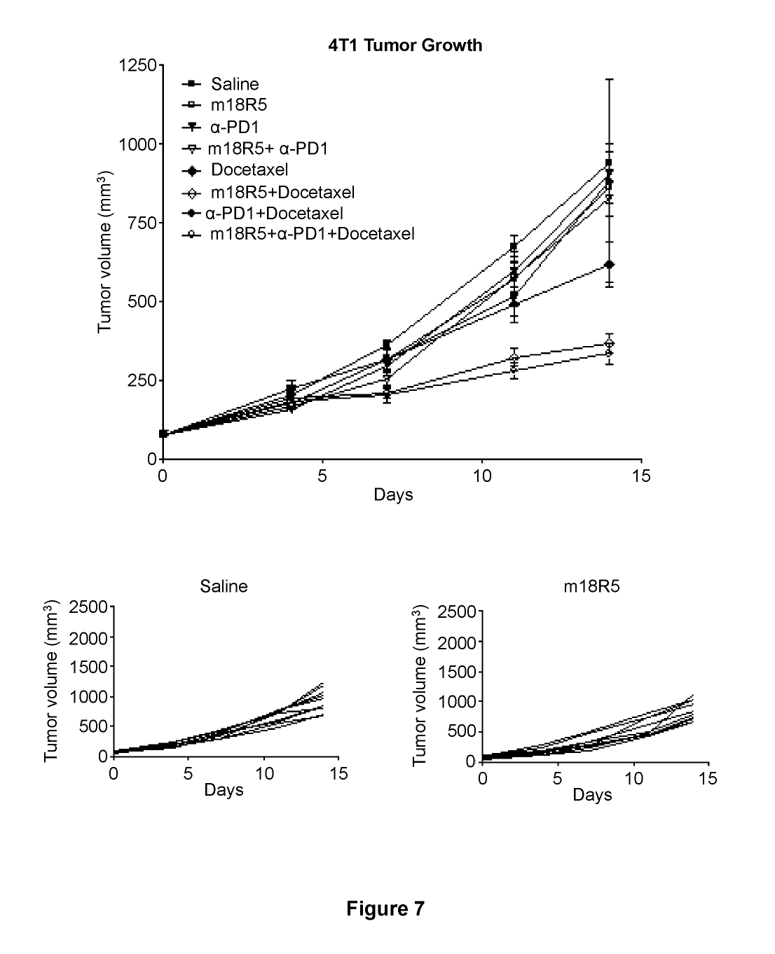

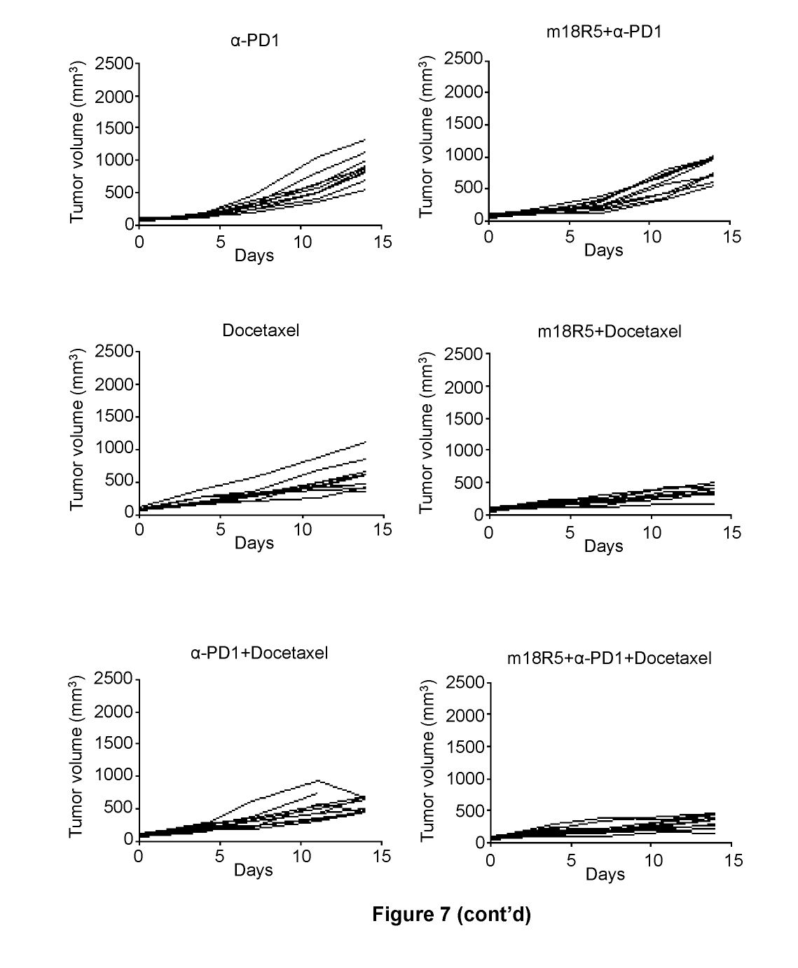

The change is T-cell tumor infiltration was also measured. Here, B16F1 tumors were isolated from control antibody, anti-CTLA-4 and anti-mouse PD-L1 combination; and anti-mouse Fzd7 (18R5)+anti-mouse anti-CTLA-4+ anti-mouse PD-L1 combination antibody-treated mice. Single cell suspensions were acquired from 4-6 independently treated tumors and were stained for tumor infiltrating immune cells using fluorescently-labeled anti-CD45, anti-CD4, and anti-CD8 antibodies. Flow cytometry analysis (FACS) was performed and the relative percentage populations of CD45+/CD4+ and CD45+/CD8+ T cells are shown. As shown in FIGS. 6A and 6B, addition of 18R5 increased CD4⁺ and CD8⁺ T cell infiltration as compared to treatment with immune checkpoint inhibitors alone.

We also observed increases in CD45+ and CD3 T cell infiltration (data not shown).

### Example 7

### Effect of Combined Anti-FZD and Anti-PD-1 Therapy in a Breast Cancer Cell Line

4T1 cells were injected subcutaneously into BALB/c mice. At a mean tumor volume of ˜80 mm³, mice were randomized for treatment with an anti-FZD antibody (murinized 18R5 (m18R5); 25 mg/kg, Q1W starting day 1), an anti-PD1 antibody (25 mg/kg, Q1W starting day 1), and/or docetaxel (33 mg/kg, Q1W starting day 4). Changes in tumor size in this experiment are shown in FIG. 16. The largest effect was observed in mice receiving m18R5+docetaxel or m18R5+anti-PD1+docetaxel.

Changes in frequency, infiltration, and activation of dendritic cells, as well as frequency of T regulatory cells were measured in 4T1 tumor-implanted mice. Selected cytokine levels (IL17a and IL2) were also measured. For these experiments, 4T1 cells were injected subcutaneously into BALB/c mice. At a mean tumor volume of ˜150 mm³, mice were randomized for treatment with the anti-FZD antibody m18R5 (25 mg/kg, day 0), an anti-PD1 antibody (25 mg/kg, day 0), and/or docetaxel (33 mg/kg, day 3). Seven days following antibody administration, mice were euthanized and tissues harvested. For the flow cytometry experiments, tumor cells were brought to a single cell suspension and the red blood cells lysed. Tumor cells were incubated with Fc block and then stained for cell surface markers shown (antibodies purchased from Affymetrix, BD Biosciences, and BioLegend), washed, and stained with fixable viability dye. Treg samples were fixed and permeabilized with Mouse Regulatory T Cell Staining Kit 1, then stained for Foxp3, per manufacturer's instructions (Affymetrix). All other samples were briefly fixed with paraformaldehyde, washed, and stored in PBS until analysis on an LSR II cytometer (BD Biosciences.) To measure cytokine levels, splenocytes were pressed through a 40 μM filter using a syringe plunger. Red blood cells were then lysed to form splenocyte cell suspensions. Two-hundred thousand cells per well were plated in the presence of AH1 peptide onto IL2 and IL17a ELISpot plates, incubated overnight, and processed per manufacturer's instructions (Mabtech). Total optical density was determined using a BIOreader ELISpot reader.

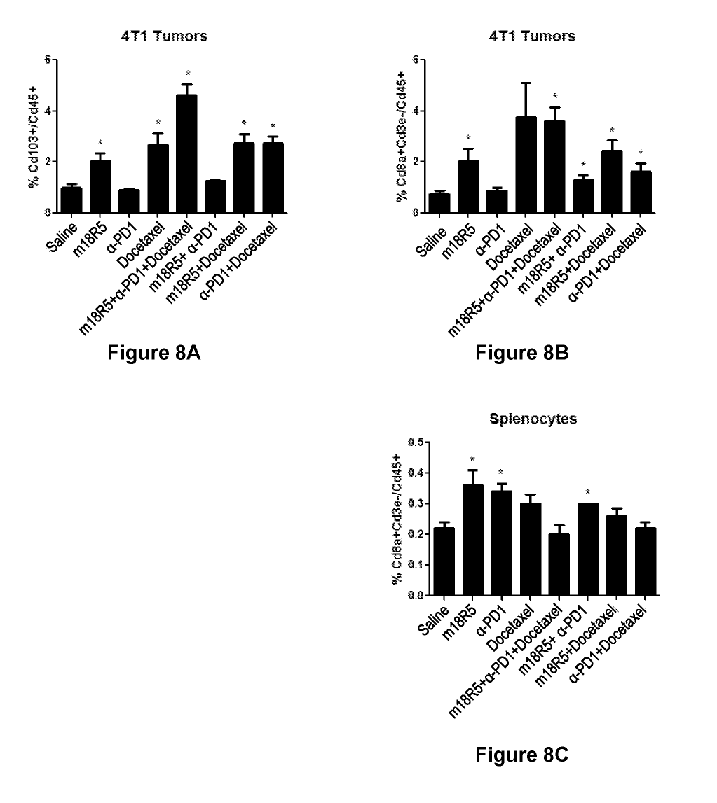

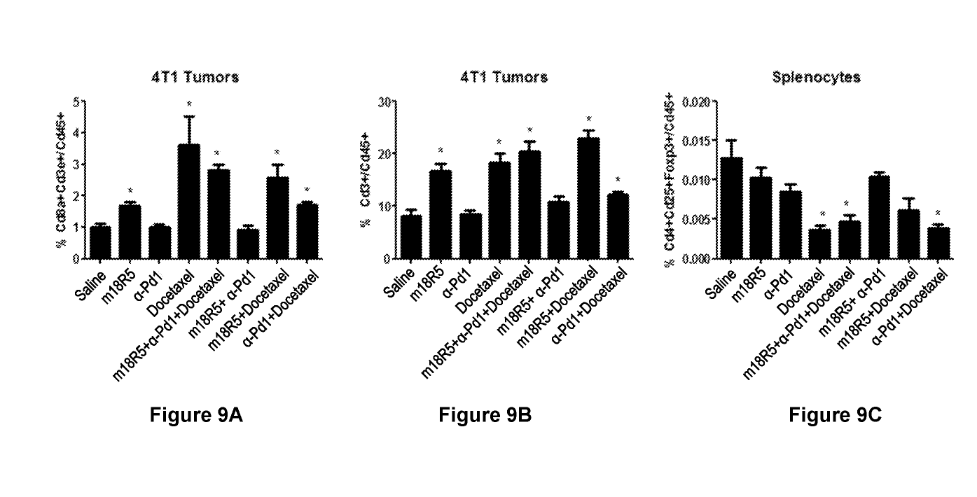

As shown in FIGS. 8A and 8B, treatment groups generally show increases in tumor dendritic cell frequency as compared to saline control. FIG. 8C shows increases in splenic dendritic cells as compared to saline control. FIGS. 9A and 9B show changes in splenic dendritic cells, and FIG. 9C shows decreases in T regulatory cells in the spleen in treatment groups as compared to saline control.

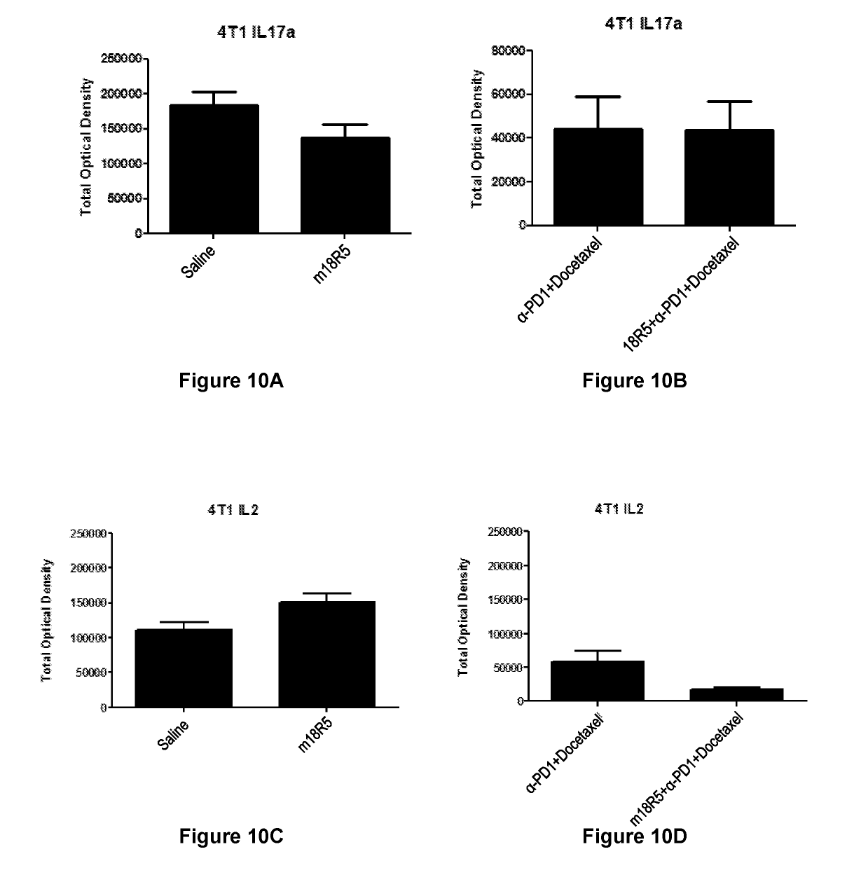

Changes in IL17a and IL2 levels were measured in control and treatment animals (FIGS. 10A-10D). m18R5 appeared to decrease IL17a levels slightly as compared to saline control (FIG. 10A), but little change was observed when comparing m18R5+anti-PD1+docetaxel to anti-PD1+docetaxel (FIG. 10B). IL2 levels were increased in mice receiving m18R5 as compared to saline (FIG. 10C), but decreased when comparing m18R5+anti-PD1+docetaxel to anti-PD1+docetaxel (FIG. 10D).

### Example 8

### Effect of Combined FZD8-Fc and Anti-PD-1 Therapy in Colon Adenocarcinoma

Murine colon adenocarcinoma MC38 cells were injected subcutaneously into C57BL/6 mice. At a mean tumor volume of ˜110 mm³, mice were randomized for treatment with 54F28 (25 mg/kg, Q1W starting day 1) and/or an anti-PD1 antibody (319R1; 25 mg/kg, Q1W starting day 1). To measure cytokine levels, splenocytes were pressed through a 40 μM filter using a syringe plunger, and red blood cells were lysed. Two-hundred thousand cells per well were plated in the presence of Adpgk peptide onto IL2 and IL17a ELISpot plates, incubated overnight, and processed per manufacturer's instructions (Mabtech). Total optical density was determined using a BIOreader ELISpot reader.

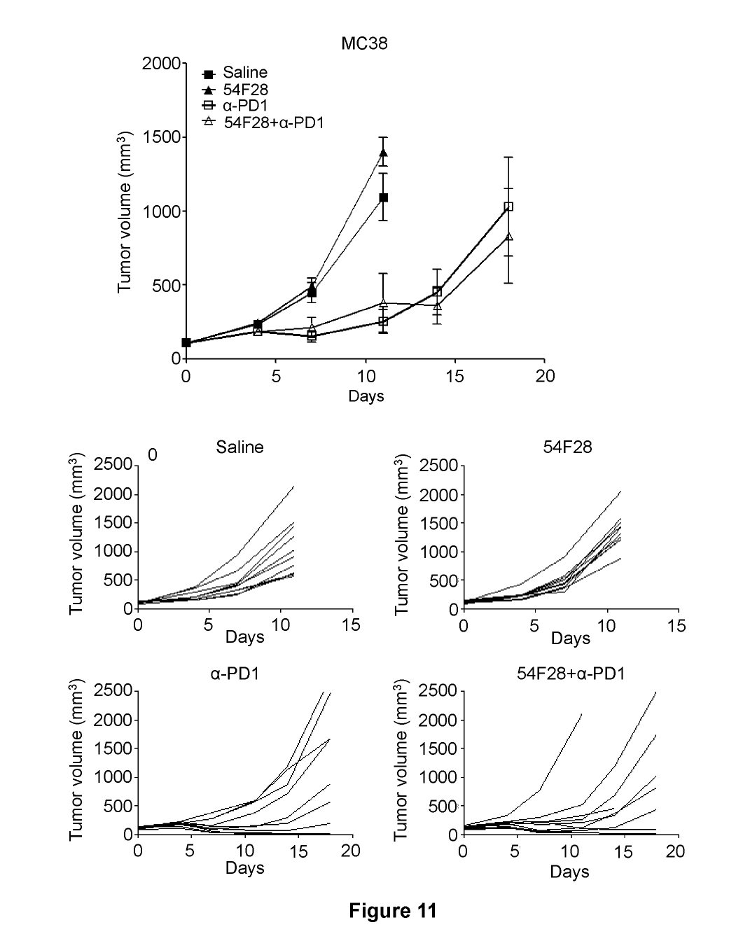

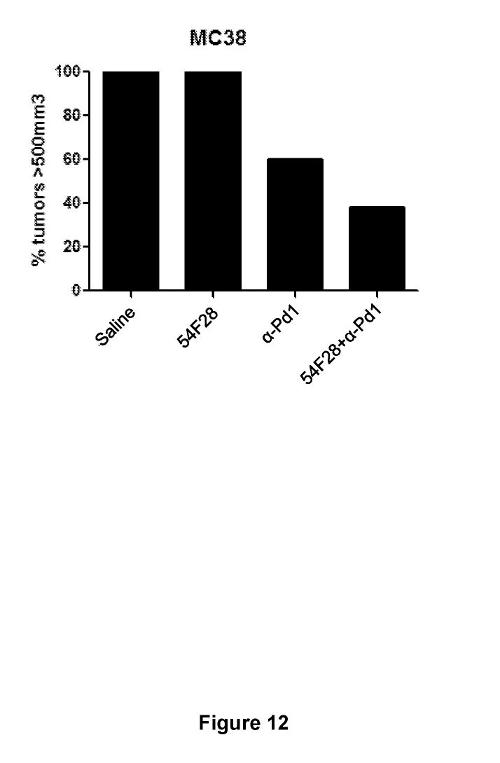

As shown in FIG. 11, the tumor growth was reduced in mice receiving the anti-PD1 antibody or receiving 54F28+anti-PD1. As shown in FIG. 12, the 54F28/anti-PD1 combination resulted in the smallest percentage of tumor samples greater than 500 mm³.

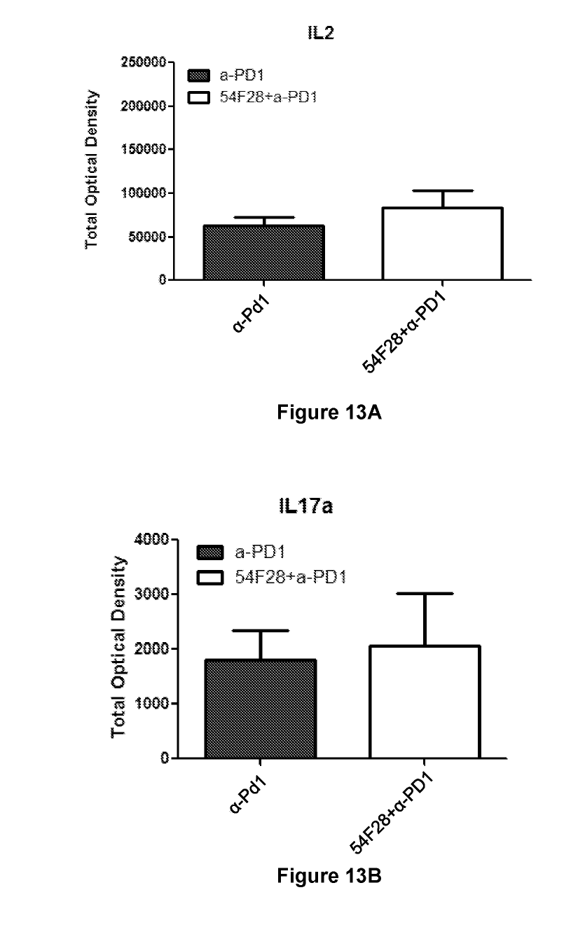

Results from cytokine measurement experiments are shown in FIGS. 13 and 13. Increases in IL2 (FIG. 13) and IL17a (FIG. 13) were observed in mice receiving anti-PD1+54F28 as compared to mice receiving anti-PD1 alone.

It is understood that the examples and embodiments described herein are for illustrative purposes only and that various modifications or changes in light thereof will be suggested to person skilled in the art and are to be included within the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

The following sequences are disclosed in the application:

OMP-18R5 Heavy chain CDR1
(SEQ ID NO: 1)
GFTFSHYTLS
OMP-18R5 Heavy chain CDR2
(SEQ ID NO: 2)
VISGDGSYTYYADSVKG
OMP-18R5 Heavy chain CDR3
(SEQ ID NO: 3)
NFIKYVFAN
OMP-18R5 Light chain CDR1
(SEQ ID NO: 4)
SGDNIGSFYVH
OMP-18R5 Light chain CDR2
(SEQ ID NO: 5)
DKSNRPSG
OMP-18R5 Light chain CDR3
(SEQ ID NO: 6)
QSYANTLSL
OMP-18R5 Heavy chain variable region amino acid sequence
(SEQ ID NO: 7)
EVQLVESGGGLVQPGGSLRLSCAASGFTFSHYTLSWVRQAPGKGLEWVSVISGDGSYTYY
ADSVKGRFTISSDNSKNTLYLQMNSLRAEDTAVYYCARNFIKYVFANWGQGTLVTVSS
OMP-18R5 Light chain variable region amino acid sequence
(SEQ ID NO: 8)
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLG
OMP-18R5 Heavy chain amino acid sequence with predicted
signal sequence underlined
(SEQ ID NO: 9)
MKHLWFFLLLVAAPRWVLSEVQLVESGGGLVQPGGSLRLSCAASGFTFSHYTLSWVRQAP
GKGLEWVSVISGDGSYTYYADSVKGRFTISSDNSKNTLYLQMNSLRAEDTAVYYCARNFI
KYVFANWGQGTLVTVSSASTKGPSVFPLAPCSRSTSESTAALGCLVKDYFPEPVTVSWNS
GALTSGVHTFPAVLQSSGLYSLSSVVTVPSSNFGTQTYTCNVDHKPSNTKVDKTVERKCC
VECPPCPAPPVAGPSVFLFPPKPKDTLMISRTPEVTCVVVDVSHEDPEVQFNWYVDGVEV
HNAKTKPREEQFNSTFRVVSVLTVVHQDWLNGKEYKCKVSNKGLPAPIEKTISKTKGQPR
EPQVYTLPPSREEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
OMP-18R5 Light chain amino acid sequence with predicted
signal sequence underlined
(SEQ ID NO: 10)
MAWALLLLTLLTQGTGSWADIELTQPPSVSVAPGQTARISCSGDNIGSFYVHWYQQKPGQ
APVLVIYDKSNRPSGIPERFSGSNSGNTATLTISGTQAEDEADYYCQSYANTLSLVFGGG
TKLTVLGQPKAAPSVTLFPPSSEELQANKATLVCLISDFYPGAVTVAWKADSSPVKAGVE
TTTPSKQSNNKYAASSYLSLTPEQWKSHRSYSCQVTHEGSTVEKTVAPTECS
OMP-18R5 Heavy chain amino acid sequence without predicted
signal sequence
(SEQ ID NO: 11)
EVQLVESGGGLVQPGGSLRLSCAASGFTFSHYTLSWVRQAPGKGLEWVSVISGDGSYTYY
ADSVKGRFTISSDNSKNTLYLQMNSLRAEDTAVYYCARNFIKYVFANWGQGTLVTVSSAS
TKGPSVFPLAPCSRSTSESTAALGCLVKDYFPEPVTVSWNSGALTSGVHTFPAVLQSSGL
YSLSSVVTVPSSNFGTQTYTCNVDHKPSNTKVDKTVERKCCVECPPCPAPPVAGPSVFLF
PPKPKDTLMISRTPEVTCVVVDVSHEDPEVQFNWYVDGVEVHNAKTKPREEQFNSTFRVV
SVLTVVHQDWLNGKEYKCKVSNKGLPAPIEKTISKTKGQPREPQVYTLPPSREEMTKNQV
SLTCLVKGFYPSDIAVEWESNGQPENNYKTTPPMLDSDGSFFLYSKLTVDKSRWQQGNVF
SCSVMHEALHNHYTQKSLSLSPGK
OMP-18R5 Light chain amino acid sequence without predicted
signal sequence
(SEQ ID NO: 12)
DIELTQPPSVSVAPGQTARISCSGDNIGSFYVHWYQQKPGQAPVLVIYDKSNRPSGIPER
FSGSNSGNTATLTISGTQAEDEADYYCQSYANTLSLVFGGGTKLTVLGQPKAAPSVTLFP
PSSEELQANKATLVCLISDFYPGAVTVAWKADSSPVKAGVETTTPSKQSNNKYAASSYLS
LTPEQWKSHRSYSCQVTHEGSTVEKTVAPTECS
Human FZD1 Fri domain amino acid sequence without predicted
signal sequence
(SEQ ID NO: 13)
QQPPPPPQQQQSGQQYNGERGISVPDHGYCQPISIPLCTDIAYNQTIMPNLLGHTNQEDA
GLEVHQFYPLVKVQCSAELKFFLCSMYAPVCTVLEQALPPCRSLCERARQGCEALMNKFG
FQWPDTLKCEKFPVHGAGELCVGQNTSDKGT
Human FZD2 Fri domain amino acid sequence without predicted
signal sequence
(SEQ ID NO: 14)
QFHGEKGISIPDHGFCQPISIPLCTDIAYNQTIMPNLLGHTNQEDAGLEVHQFYPLVKVQ
CSPELRFFLCSMYAPVCTVLEQAIPPCRSICERARQGCEALMNKFGFQWPERLRCEHFPR
HGAEQICVGQNHSEDG
Human FZD3 Fri domain amino acid sequence without predicted
signal sequence
(SEQ ID NO: 15)
HSLFSCEPITLRMCQDLPYNTTFMPNLLNHYDQQTAALAMEPFHPMVNLDCSRDF
RPFLCALYAPICMEYGRVTLPCRRLCQRAYSECSKLMEMFGVPWPEDMECSRFPDCDEPY
PRLVDL
Human FZD4 Fri domain amino acid sequence without predicted
signal sequence
(SEQ ID NO: 16)
FGDEEERRCDPIRISMCQNLGYNVTKMPNLVGHELQTDAELQLTTFTPLIQYGCSSQLQF
FLCSVYVPMCTEKINIPIGPCGGMCLSVKRRCEPVLKEFGFAWPESLNCSKFPPQNDHNH
MCMEGPGDEEV
Human FZD5 Fri domain amino acid sequence without predicted
signal sequence
(SEQ ID NO: 17)
ASKAPVCQEITVPMCRGIGYNLTHMPNQFNHDTQDEAGLEVHQFWPLVEIQCSPDLRFFL
CSMYTPICLPDYHKPLPPCRSVCERAKAGCSPLMRQYGFAWPERMSCDRLPVLGRDAEVL
CMDYNRSEATT
Human FZD6 Fri domain amino acid sequence without predicted
signal sequence
(SEQ ID NO: 18)
HSLFTCEPITVPRCMKMAYNMTFFPNLMGHYDQSIAAVEMEHFLPLANLECSPNIETFLC
KAFVPTCIEQIHVVPPCRKLCEKVYSDCKKLIDTFGIRWPEELECDRLQYCDETVPVTFD
PHTEFLG
Human FZD7 Fri domain amino acid sequence without predicted
signal sequence
(SEQ ID NO: 19)
QPYHGEKGISVPDHGFCQPISIPLCTDIAYNQTILPNLLGHTNQEDAGLEVHQFYPLVKV
QCSPELRFFLCSMYAPVCTVLDQAIPPCRSLCERARQGCEALMNKFGFQWPERLRCENFP
VHGAGEICVGQNTSDGSG
Human FZD8 Fri domain amino acid sequence without predicted
signal sequence
(SEQ ID NO: 20)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTT
Human FZD9 Fri domain amino acid sequence without predicted
signal sequence
(SEQ ID NO: 21)
LEIGRFDPERGRGAAPCQAVEIPMCRGIGYNLTRMPNLLGHTSQGEAAAELAEFAPLVQY
GCHSHLRFFLCSLYAPMCTDQVSTPIPACRPMCEQARLRCAPIMEQFNFGWPDSLDCARL
PTRNDPHALCMEAPENA
Human FZD10 Fri domain amino acid sequence without predicted
signal sequence
(SEQ ID NO: 22)
ISSMDMERPGDGKCQPIEIPMCKDIGYNMTRMPNLMGHENQREAAIQLHEFAPLVEYGCH
GHLRFFLCSLYAPMCTEQVSTPIPACRVMCEQARLKCSPIMEQFNFKWPDSLDCRKLPNK
NDPNYLCMEAPNNG
Human FZD1 amino acids 116-227
(SEQ ID NO: 23)
CQPISIPLCTDIAYNQTIMPNLLGHTNQEDAGLEVHQFYPLVKVQCSAELKFFLCSMYAP
VCTVLEQALPPCRSLCERARQGCEALMNKFGFQWPDTLKCEKFPVHGAGELC
Human FZD2 amino acids 39-150
(SEQ ID NO: 24)
CQPISIPLCTDIAYNQTIMPNLLGHTNQEDAGLEVHQFYPLVKVQCSPELRFFLCSMYAP
VCTVLEQAIPPCRSICERARQGCEALMNKFGFQWPERLRCEHFPRHGAEQIC
Human FZD3 amino acids 28-133
(SEQ ID NO: 25)
CEPITLRMCQDLPYNTTFMPNLLNHYDQQTAALAMEPFHPMVNLDCSRDFRPFLCALYAP
ICMEYGRVTLPCRRLCQRAYSECSKLMEMFGVPWPEDMECSRFPDC
Human FZD4 amino acids 48-161
(SEQ ID NO: 26)
CDPIRISMCQNLGYNVTKMPNLVGHELQTDAELQLTTFTPLIQYGCSSQLQFFLCSVYVP
MCTEKINIPIGPCGGMCLSVKRRCEPVLKEFGFAWPESLNCSKFPPQNDHNHMC
Human FZD5 amino acids 33-147
(SEQ ID NO: 27)
CQEITVPMCRGIGYNLTHMPNQFNHDTQDEAGLEVHQFWPLVEIQCSPDLRFFLCSMYTP
ICLPDYHKPLPPCRSVCERAKAGCSPLMRQYGFAWPERMSCDRLPVLGRDAEVLC
Human FZD6 amino acids 24-129
(SEQ ID NO: 28)
CEPITVPRCMKMAYNMTFFPNLMGHYDQSIAAVEMEHFLPLANLECSPNIETFLCKAFVP
TCIEQIHVVPPCRKLCEKVYSDCKKLIDTFGIRWPEELECDRLQYC
Human FZD7 amino acids 49-160
(SEQ ID NO: 29)
CQPISIPLCTDIAYNQTILPNLLGHTNQEDAGLEVHQFYPLVKVQCSPELRFFLCSMYAP
VCTVLDQAIPPCRSLCERARQGCEALMNKFGFQWPERLRCENFPVHGAGEIC
Human FZD8 amino acids 35-148
(SEQ ID NO: 30)
CQEITVPLCKGIGYNYTYMPNQFNHDTQDEAGLEVHQFWPLVEIQCSPDLKFFLCSMYTP
ICLEDYKKPLPPCRSVCERAKAGCAPLMRQYGFAWPDRMRCDRLPEQGNPDTLC
Human FZD9 amino acids 39-152
(SEQ ID NO: 31)
CQAVEIPMCRGIGYNLTRMPNLLGHTSQGEAAAELAEFAPLVQYGCHSHLRFFLCSLYAP
MCTDQVSTPIPACRPMCEQARLRCAPIMEQFNFGWPDSLDCARLPTRNDPHALC
Human FZD10 amino acids 34-147
(SEQ ID NO: 32)
CQPIEIPMCKDIGYNMTRMPNLMGHENQREAAIQLHEFAPLVEYGCHGHLRFFLCSLYAP
MCTEQVSTPIPACRVMCEQARLKCSPIMEQFNFKWPDSLDCRKLPNKNDPNYLC
Human FZD8 Fri domain amino acid sequence without predicted
signal sequence (variant)
(SEQ ID NO: 33)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDL
Human IgG₁ Fc region
(SEQ ID NO: 34)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
Human IgG₁ Fc region (variant)
(SEQ ID NO: 35)
DKTHTCPPCPAPELLGGPSVFLFPPKPKDTLMISRTPEVTCVVVDVSHEDPEVKFNWYVD
GVEVHNAKTKPREEQYNSTYRVVSVLTVLHQDWLNGKEYKCKVSNKALPAPIEKTISKAK
GQPREPQVYTLPPSREEMTKNQVSLTCLVKGFYPSDIAVEWESNGQPENNYKTTPPVLDS
DGSFELYSKLTVDKSRWQQGNVFSCSVMHEALHNHYTQKSLSLSPGK
Human IgG₁ Fc region
(SEQ ID NO: 36)
KSSDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTIS
KAKGQPREPQVYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
Human IgG₁ Fc region
(SEQ ID NO: 37)
EPKSSDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKT
ISKAKGQPREPQVYTLPPSRDELTKNQVSLTCLVKGFYPSDIAVEWESNGQPENNYKTTP
PVLDSDGSFFLYSKLTVDKSRWQQGNVFSCSVMHEALHNHYTQKSLSLSPGK
Human IgG₂ Fc region
(SEQ ID NO: 38)
CVECPPCPAPPVAGPSVFLFPPKPKDTLMISRTPEVTCVVVDVSHEDPEVQFNWYVDGVE
VHNAKTKPREEQFNSTFRVVSVLTVVHQDWLNGKEYKCKVSNKGLPAPIEKTISKTKGQP
REPQVYTLPPSREEMTKNQVSLTCLVKGFYPSDIAVEWESNGQPENNYKTTPPMLDSDGS
FFLYSKLTVDKSRWQQGNVFSCSVMHEALHNHYTQKSLSLSPGK
FZD8-Fc variant 54F03 amino acid sequence (without predicted
signal sequence)
(SEQ ID NO: 39)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTGRADKTHTCPPCPAPELLGGPSVFLFPPKPKDTLMISRTPEVICVVVDV
SHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
K
FZD8-Fc variant 54F16, 54F17, 54F18, 54F23, 54F25, 54F27,
54F29, 54F31, and 54F34 amino acid sequence (without
predicted signal sequence)
(SEQ ID NO: 40)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTKSSDKTHTCPPCPAPELLGGPSVFLFPPKPKDTLMISRTPEVTCVVVDV
SHEDPEVKFNWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLSLSPG
K
FZD8-Fc variant 54F19, 54F20, 54F24, 54F26, 54F28, 54F30,
54F32, 54F34 and 54F35 amino acid sequence (without
predicted signal sequence)
(SEQ ID NO: 41)
ASAKELACQEITVPLCKGIGYNYTYMPNQFNHDTQDEAGLEVHQFWPLVEIQCSPDLKFF
LCSMYTPICLEDYKKPLPPCRSVCERAKAGCAPLMRQYGFAWPDRMRCDRLPEQGNPDTL
CMDYNRTDLTTEPKSSDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDWLNGKEYKCKVS
NKALPAPIEKTISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHNHYTQKSLSLS
PGK
FZD8-Fc variant 54F03 amino acid sequence with signal
sequence
(SEQ ID NO: 42)
MEWGYLLEVTSLLAALALLQRSSGAAAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTGRADKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
FZD8-Fc variant 54F16 amino acid sequence with signal
sequence
(SEQ ID NO: 43)
MEWGYLLEVTSLLAALALLQRSSGAAAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTKSSDKTHTCPPCPAPELLGGPS
VFLFPPKPKDTLMISRTPEVTCVVVDVSHEDPEVKFNWYVDGVEVHNAKTKPREEQYNST
YRVVSVLTVLHQDWLNGKEYKCKVSNKALPAPIEKTISKAKGQPREPQVYTLPPSRDELT
KNQVSLTCLVKGFYPSDIAVEWESNGQPENNYKTTPPVLDSDGSFFLYSKLTVDKSRWQQ
GNVFSCSVMHEALHNHYTQKSLSLSPGK
FZD8-Fc variant 54F26 with signal sequence
(SEQ ID NO: 44)
MEWGYLLEVTSLLAALFLLQRSPIVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRTDLTTEPKSSDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
FZD8-Fc variant 54F28 with signal sequence
(SEQ ID NO: 45)
MEWGYLLEVTSLLAALLLLQRSPFVHAASAKELACQEITVPLCKGIGYNYTYMPNQFNHD
TQDEAGLEVHQFWPLVEIQCSPDLKFFLCSMYTPICLEDYKKPLPPCRSVCERAKAGCAP
LMRQYGFAWPDRMRCDRLPEQGNPDTLCMDYNRIDLTTEPKSSDKTHTCPPCPAPELLGG
PSVFLFPPKPKDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLYSKLTVDKSRW
QQGNVFSCSVMHEALHNHYTQKSLSLSPGK
Human Wnt1 C-terminal cysteine rich domain (aa 288-370)
(SEQ ID NO: 46)
DLVYFEKSPNFCTYSGRLGTAGTAGRACNSSSPALDGCELLCCGRGHRTRTQRVTERCNC
TFHWCCHVSCRNCTHTRVLHECL
Human Wnt2 C-terminal cysteine rich domain (aa 267-360)
(SEQ ID NO: 47)
DLVYFENSPDYCIRDREAGSLGTAGRVCNLTSRGMDSCEVMCCGRGYDTSHVTRMTKCGC
KFHWCCAVRCQDCLEALDVHTCKAPKNADWTTAT
Human Wnt2b C-terminal cysteine rich domain (aa 298-391)
(SEQ ID NO: 48)
DLVYFDNSPDYCVLDKAAGSLGTAGRVCSKTSKGTDGCEIMCCGRGYDTTRVTRVTQCEC
KFHWCCAVRCKECRNTVDVHTCKAPKKAEWLDQT
Human Wnt3 C-terminal cysteine rich domain (aa 273-355)
(SEQ ID NO: 49)
DLVYYENSPNFCEPNPETGSFGTRDRTCNVTSHGIDGCDLLCCGRGHNTRTEKRKEKCHC
IFHWCCYVSCQECIRIYDVHTCK
Human Wnt3a C-terminal cysteine rich domain (aa 270-352)
(SEQ ID NO: 50)
DLVYYEASPNFCEPNPETGSFGTRDRTCNVSSHGIDGCDLLCCGRGHNARAERRREKCRC
VFHWCCYVSCQECTRVYDVHTCK
Human Wnt7a C-terminal cysteine rich domain (aa 267-359)
(SEQ ID NO: 51)
DLVYIEKSPNYCEEDPVTGSVGTQGRACNKTAPQASGCDLMCCGRGYNTHQYARVWQCNC
KFHWCCYVKCNTCSERTEMYTCK
Human Wnt7b C-terminal cysteine rich domain (aa 267-349)
(SEQ ID NO: 52)
DLVYIEKSPNYCEEDAATGSVGTQGRLCNRTSPGADGCDTMCCGRGYNTHQYTKVWQCNC
KFHWCCFVKCNTCSERTEVFTCK
Human Wnt8a C-terminal cysteine rich domain (aa 248-355)
(SEQ ID NO: 53)
ELIFLEESPDYCTCNSSLGIYGTEGRECLQNSHNTSRWERRSCGRLCTECGLQVEERKTE
VISSCNCKFQWCCTVKCDQCRHVVSKYYCARSPGSAQSLGRVWFGVYI
Human Wnt8b C-terminal cysteine rich domain (aa 245-351)
(SEQ ID NO: 54)
ELVHLEDSPDYCLENKTLGLLGTEGRECLRRGRALGRWELRSCRRLCGDCGLAVEERRAE
TVSSCNCKFHWCCAVRCEQCRRRVTKYFCSRAERPRGGAAHKPGRKP
Human Wnt10a C-terminal cysteine rich domain (aa 335-417)
(SEQ ID NO: 55)
DLVYFEKSPDFCEREPRLDSAGTVGRLCNKSSAGSDGCGSMCCGRGHNILRQTRSERCHC
RFHWCCFVVCEECRITETAWSVCK
Human Wnt10b C-terminal cysteine rich domain (aa 307-389)
(SEQ ID NO: 56)
ELVYFEKSPDFCERDPTMGSPGTRGRACNKTSRLLDGCGSLCCGRGHNVLRQTRVERCHC
RFHWCCYVLCDECKVTEWVNVCK
Linker
(SEQ ID NO: 57)
ESGGGGVT
Linker
(SEQ ID NO: 58)
LESGGGGVT
Linker
(SEQ ID NO: 59)
GRAQVT
Linker
(SEQ ID NO: 60)
WRAQVT
Linker
(SEQ ID NO: 61)
ARGRAQVT

What is claimed is:

1. A method of treating cancer comprising administering to a subject a therapeutically effective amount of a Wnt pathway inhibitor and a therapeutically effective amount of an immunotherapeutic agent.

2. A method of inhibiting tumor growth in a subject, wherein the method comprises administering to a subject a therapeutically effective amount of a Wnt pathway inhibitor and an immunotherapeutic agent.

3. A method of inhibiting the activity of regulatory T-cells (Tregs), wherein the method comprises administering to a subject a therapeutically effective amount of a Wnt pathway inhibitor and a therapeutically effective amount an immunotherapeutic agent.

4. A method of increasing T cell infiltration into a tumor, wherein the method comprises administering to a subject a therapeutically effective amount of a Wnt pathway inhibitor and a therapeutically effective amount of an immunotherapeutic agent.

5. A method of increasing T cell cytotoxicity to a tumor, wherein the method comprises administering to a subject a therapeutically effective amount of a Wnt pathway inhibitor and a therapeutically effective amount of an immunotherapeutic agent.

6. A method of increasing tumor cell lysis, wherein the method comprises administering to a subject a therapeutically effective amount of a Wnt pathway inhibitor and a therapeutically effective amount of an immunotherapeutic agent.

7. A method to increase the efficacy of an immune checkpoint modulator, wherein the method comprises administering to a subject a therapeutically effective amount of a Wnt pathway inhibitor in combination with the immune checkpoint modulator.

8. A method of reducing or preventing cancer metastasis in a subject, wherein the method comprises administering to the subject a therapeutically effective amount of a Wnt pathway inhibitor and a therapeutically effective amount of an immunotherapeutic agent.

9. The method according to any one of claims 1-8, wherein the Wnt pathway inhibitor is an antibody.

10. The method according to any one of claims 1-9, wherein the Wnt pathway inhibitor is an antibody that specifically binds at least one frizzled (FZD) protein or portion thereof.

11. The method of claim 9 or claim 10, wherein the antibody specifically binds at least one FZD protein selected from the group consisting of: FZD1, FZD2, FZD3, FZD4, FZD5, FZD6, FZD7, FZD8, FZD9, and FZD10.

12. The method according to any one of claims 9-11, wherein the antibody specifically binds FZD1, FZD2, FZD5, FZD7, and/or FZD8.

13. The method according to any one of claim 9-12, wherein the antibody comprises:
(a) a heavy chain CDR1 comprising GFTFSHYTLS (SEQ ID NO:1), a heavy chain CDR2 comprising VISGDGSYTYYADSVKG (SEQ ID NO:2), and a heavy chain CDR3 comprising NFIKYVFAN (SEQ ID NO:3), and
(b) a light chain CDR1 comprising SGDNIGSFYVH (SEQ ID NO:4), a light chain CDR2 comprising DKSNRPSG (SEQ ID NO:5), and a light chain CDR3 comprising QSYANTLSL (SEQ ID NO:6).

14. The method according to any one of claims 9-13, wherein the antibody comprises:
(a) a heavy chain variable region having at least 90% sequence identity to SEQ ID NO:7; and/or
(b) a light chain variable region having at least 90% sequence identity to SEQ ID NO:8.

15. The method according to any one of claims 9-13, wherein the antibody comprises:
(a) a heavy chain variable region having at least 95% sequence identity to SEQ ID NO:7; and/or
(b) a light chain variable region having at least 95% sequence identity to SEQ ID NO:8.

16. The method according to any one of claims 9-15, wherein the antibody comprises:
(a) a heavy chain variable region comprising SEQ ID NO:7; and/or
(b) a light chain variable region comprising SEQ ID NO:8.

17. The method according to any one of claims 9-16, wherein the antibody comprises:
(a) a heavy chain variable region consisting essentially of SEQ ID NO:7; and
(b) a light chain variable region consisting essentially of SEQ ID NO:8.

18. The method according to any one of claims 9-17, wherein the antibody comprises:
(a) a heavy chain consisting essentially of SEQ ID NO:9 or SEQ ID NO:11; and
(b) a light chain consisting essentially of SEQ ID NO:10 or SEQ ID NO:12.

19. The method according to any one of claims 9-18, wherein the antibody is 18R5.

20. The method according to any one of claims 1-8, wherein the Wnt pathway inhibitor is a Wnt-binding agent.

21. The method of claim 20, wherein the Wnt-binding agent is an antibody.

22. The method according to any one of claims 1-8 and 21, wherein the Wnt pathway inhibitor is an antibody that specifically binds at least one Wnt protein.

23. The method of claim 22, wherein the antibody specifically binds at least one Wnt protein selected from the group consisting of: Wnt1, Wnt2, Wnt2b, Wnt3, Wnt3a, Wnt7a, Wnt7b, Wnt8a, Wnt8b, Wnt10a, and Wnt10b.

24. The method according to any one of claims 9-19 and 21-23, wherein the antibody is a monoclonal antibody, a recombinant antibody, a chimeric antibody, a humanized antibody, a human antibody, or an antibody fragment comprising an antigen-binding site.

25. The method according to any one of claims 9-19 and 21-24, wherein the antibody is a monospecific antibody or a bispecific antibody.

26. The method according to any one of claims 9-19 and 21-25, wherein the antibody is an IgG1 antibody or an IgG2 antibody.

27. The method according to any one of claims 1-8, wherein the Wnt pathway inhibitor is a soluble receptor.

28. The method of claim 20, wherein the Wnt-binding agent is a soluble receptor.

29. The method of claim 27 or claim 28, wherein the soluble receptor comprises a Fri domain of a human FZD protein.

30. The method of claim 29, wherein the Fri domain of the human FZD protein consists essentially of: Fri domain of FZD1, Fri domain of FZD2, Fri domain of FZD3, Fri domain of FZD4, Fri domain of FZD5, Fri domain of FZD6, Fri domain of FZD7, Fri domain of FZD8, Fri domain of FZD9, or Fri domain of FZD10.

31. The method of claim 30, wherein the Fri domain of the human FZD protein consists essentially of the Fri domain of FZD8.

32. The method of claim 29, wherein the Fri domain of the human FZD protein comprises a sequence selected from the group consisting of: SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, and SEQ ID NO:33.

33. The method of claim 32, wherein the Fri domain of the human FZD protein consists essentially of SEQ ID NO:20 or SEQ ID NO:33.

34. The method according to any one of claims 29-33, wherein the Fri domain of the human FZD protein is directly linked to a non-FZD polypeptide.

35. The method according to any one of claims 29-33, wherein the Fri domain of the human FZD protein is connected to a non-FZD polypeptide by a linker.

36. The method of claim 34 or claim 35, wherein the non-FZD polypeptide comprises a human Fc region.

37. The method according to any one of claims 31-33, wherein the non-FZD polypeptide consists essentially of SEQ ID NO:34, SEQ ID NO:35, SEQ ID NO:36, SEQ ID NO:37, or SEQ ID NO:38.

38. The method of claim 20, wherein the Wnt-binding agent comprises:
(a) a first polypeptide consisting essentially of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and
(b) a second polypeptide consisting essentially of SEQ ID NO:34, SEQ ID NO:35, SEQ ID NO:36, SEQ ID NO:37, or SEQ ID NO:38;
wherein the first polypeptide is directly linked to the second polypeptide.

39. The method of claim 20, wherein the Wnt-binding agent comprises:
(a) a first polypeptide consisting essentially of SEQ ID NO:13, SEQ ID NO:14, SEQ ID NO:15, SEQ ID NO:16, SEQ ID NO:17, SEQ ID NO:18, SEQ ID NO:19, SEQ ID NO:20, SEQ ID NO:21, SEQ ID NO:22, SEQ ID NO:23, SEQ ID NO:24, SEQ ID NO:25, SEQ ID NO:26, SEQ ID NO:27, SEQ ID NO:28, SEQ ID NO:29, SEQ ID NO:30, SEQ ID NO:31, SEQ ID NO:32, or SEQ ID NO:33; and
(b) a second polypeptide consisting essentially of SEQ ID NO:34, SEQ ID NO:35, SEQ ID NO:36, SEQ ID NO:37, or SEQ ID NO:38;
wherein the first polypeptide is connected to the second polypeptide by a linker.

40. The method of claim 38 or claim 39, wherein the first polypeptide consists essentially of SEQ ID NO:20.

41. The method of claim 38 or claim 39, wherein the first polypeptide consists essentially of SEQ ID NO:20, and wherein the second polypeptide consists essentially of SEQ ID NO:36, SEQ ID NO:37, or SEQ ID NO:35.

42. The method of claim 38 or claim 39, wherein the first polypeptide consists essentially of SEQ ID NO:33.

43. The method of claim 38 or claim 39, wherein the first polypeptide consists essentially of SEQ ID NO:33, and wherein the second polypeptide consists essentially of SEQ ID NO:36, SEQ ID NO:37, or SEQ ID NO:35.

44. The method of claim 20, wherein the Wnt-binding agent comprises SEQ ID NO:39, SEQ ID NO:40, or SEQ ID NO:41.

45. The method of claim 20, wherein the Wnt-binding agent comprises SEQ ID NO:41.

46. The method of claim 20, wherein the Wnt-binding agent is 54F28.

47. The method of any one of claims 1-46, wherein the Wnt pathway inhibitor enhances the activity of the immunotherapeutic agent.

48. The method of any one of claims 1-46, wherein the immunotherapeutic agent enhances the activity of the Wnt pathway inhibitor.

49. The method of any one of claims 1-46, wherein the Wnt pathway inhibitor and the immunotherapeutic agent act synergistically.

50. The method of any one of claim 1-6 or 8-49, wherein the immunotherapeutic agent selected from a group consisting of: a modulator of PD-1 activity, a modulator of PD-L1 activity, a modulator of PD-L2 activity, a modulator of CTLA-4 activity, a modulator of CD28 activity, a modulator of CD80 activity, a modulator of CD86 activity, a modulator of 4-1BB activity, an modulator of OX40 activity, a modulator of KIR activity, a modulator of Tim-3 activity, a modulator of LAG3 activity, a modulator of CD27 activity, a modulator of CD40 activity, a modulator of GITR activity, a modulator of TIGIT activity, a modulator of CD20 activity, a modulator of CD96 activity, a modulator of IDO1 activity, a cytokine, a chemokine, an interferon, an interleukin, a lymphokine, a member of the tumor necrosis factor (TNF) family, and an immunostimulatory oligonucleotide.

51. The method of any one of claim 1-6 or 8-50, wherein the immunotherapeutic agent is an immune checkpoint modulator.

52. The method of claim 7 or claim 51, wherein the immune checkpoint modulator is an immune checkpoint inhibitor.

53. The method of claim 52, wherein the immune checkpoint inhibitor is a PD-1 antagonist, a PD-L1 antagonist, a PD-L2 antagonist, a CTLA-4 antagonist, a CD80 antagonist, a CD86 antagonist, a KIR antagonist, a Tim-3 antagonist, a LAG3 antagonist, a TIGIT antagonist, a CD20 antagonist, a CD96 antagonist, or a IDO1 antagonist.

54. The method of claim 53, wherein the PD-1 antagonist is an antibody that specifically binds PD-1.

55. The method of claim 54, wherein the antibody that binds PD-1 is pembrolizumab (KEYTRUDA; MK-3475), pidilizumab (CT-011), nivolumab (OPDIVO; BMS-936558), MEDI0680 (AMP-514), REGN2810, BGB-A317, PDR-001, or STI-A1110.

56. The method of claim 53, wherein the PD-1 antagonist comprises the extracellular domain of PD-L2.

57. The method of claim 56, wherein the PD-1 antagonist is AMP-224.

58. The method of claim 53, wherein the PD-1 antagonist is a peptide.

59. The method of claim 58, wherein the PD-1 antagonist is AUNP-12.

60. The method of claim 53, wherein the PD-L1 antagonist is an antibody that specifically binds PD-L1.

61. The method of claim 60, wherein the antibody that binds PD-L1 is atezolizumab (RG7446; MPDL3280A), MEDI4736, BMS-936559 (MDX-1105), avelumab (MSB0010718C), KD033, the antibody portion of KD033, or STI-A1014.

62. The method of claim 53, wherein the CTLA-4 antagonist is an antibody that specifically binds CTLA-4.

63. The method of claim 62, wherein the antibody that binds CTLA-4 is ipilimumab (YERVOY; MDX-010, BMS-734016) or tremelimumab (CP-675,206; ticilimumab).

64. The method of claim 53, wherein the CTLA-4 antagonist comprises a soluble CTLA-4 receptor.

65. The method of claim 64, wherein the CTLA-4 antagonist is KAHR-102.

66. The method of claim 53, wherein the LAG3 antagonist is an antibody that specifically binds LAG3.

67. The method of claim 66, wherein the antibody that binds LAG3 is IMP701, BMS-986016, LAG525, GSK2831781, or IMP731.

68. The method of claim 53, wherein the LAG3 antagonist comprises a soluble LAG3 receptor.

69. The method of claim 68, wherein the LAG3 antagonist is IMP321.

70. The method of claim 53, wherein the Tim-3 antagonist is an antibody that binds Tim-3.

71. The method of claim 53, wherein the TIGIT antagonist is an antibody that binds TIGIT.

72. The method of claim 53, wherein the KIR antagonist is an antibody that specifically binds KIR.

73. The method of claim 72, wherein the antibody that binds KIR is lirilumab.

74. The method of claim 51, wherein the immune checkpoint modulator is an immune checkpoint enhancer or stimulator.

75. The method of claim 74, wherein the immune checkpoint enhancer or stimulator is a CD28 agonist, a 4-1BB agonist, an OX40 agonist, a CD27 agonist, a CD80 agonist, a CD86 agonist, a CD40 agonist, or a GITR agonist.

76. The method of claim 75, wherein the OX40 agonist comprises OX40 ligand, or an OX40-binding portion thereof.

77. The method of claim 76, wherein the OX40 agonist is MEDI6383.

78. The method of claim 75, wherein the OX40 agonist is an antibody that specifically binds OX40.

79. The method of claim 78, wherein the antibody that binds OX40 is MEDI6469, MEDI0562, or MOXR0916 (RG7888).

80. The method of claim 75, wherein the OX40 agonist is a vector capable of expressing OX40 ligand.

81. The method of claim 75, wherein the OX40 agonist is Delta-24-RGDOX or DNX2401.

82. The method of claim 75, wherein the 4-1BB agonist is PRS-343.

83. The method of claim 75, wherein the 4-1BB agonist is an antibody that specifically binds 4-1BB.

84. The method of claim 84, wherein the antibody that binds 4-1BB is PF-2566 (PF-05082566) or urelumab (BMS-663513).

85. The method of claim 75, wherein the CD27 agonist is an antibody that specifically binds CD27.

86. The method of claim 85, wherein the antibody that binds CD27 is varlilumab (CDX-1127).

87. The method of claim 75, wherein the GITR agonist comprises GITR ligand or a GITR-binding portion thereof.

88. The method of claim 75, wherein the GITR agonist is an antibody that specifically binds GITR.

89. The method of claim 88, wherein the antibody that binds GITR is TRX518, MK-4166, or INBRX-110.

90. The method of any one of claims 1-50, wherein the immunotherapeutic agent is a cytokine.

91. The method of claim 90, wherein the cytokine is a chemokine, an interferon, an interleukin, lymphokine, or a member of the tumor necrosis factor family.

92. The method of claim 91, wherein the cytokine is IL-2, IL15, or interferon-gamma.

93. The method of any one of claims 1 and 8-92, wherein the cancer is selected from the group consisting of lung cancer, pancreatic cancer, breast cancer, colon cancer, colorectal cancer, melanoma, gastrointestinal cancer, gastric cancer, renal cancer, ovarian cancer, liver cancer, endometrial cancer, kidney cancer, prostate cancer, thyroid cancer, neuroblastoma, glioma, glioblastoma, glioblastoma multiforme, cervical cancer, stomach cancer, bladder cancer, head and neck cancer, and hepatoma.

94. The method of any one of claims 2, 4-6, and 9-92, wherein the tumor is selected from the group consisting of lung tumor, pancreatic tumor, breast tumor, colon tumor, colorectal tumor, melanoma, gastrointestinal tumor, gastric tumor, renal tumor, ovarian tumor, liver tumor, endometrial tumor, kidney tumor, prostate tumor, thyroid tumor, neuroblastoma, glioma, glioblastoma, glioblastoma multiforme, cervical tumor, stomach tumor, bladder tumor, head and neck tumor, and hepatoma.

95. The method of any one of claims 1, 2, 4-6, and 8-94, wherein the subject's cancer or tumor does not respond to an immune checkpoint inhibitor.

96. The method of any one of claims 1, 2, 4-6, and 8-94, wherein the subject's cancer or tumor has progressed following an initial response to an immune checkpoint inhibitor.

97. The method of claim 95 or 96, where the immune checkpoint inhibitor is PD-1 antagonist or PD-L1 antagonist therapy.

98. The method of any one of claims 1-97, wherein the subject is a human.


SEQUENCE LISTING (61 sequences)

SEQ ID NO: 1 - protein, 10 aa, Artificial Sequence
  Heavy chain CDR1
GFTFSHYTLS

SEQ ID NO: 2 - protein, 17 aa, Artificial Sequence
  Heavy chain CDR2
VISGDGSYTYYADSVKG

SEQ ID NO: 3 - protein, 9 aa, Artificial Sequence
  Heavy chain CDR3
NFIKYVFAN

SEQ ID NO: 4 - protein, 11 aa, Artificial Sequence
  Light chain CDR1
SGDNIGSFYVH

SEQ ID NO: 5 - protein, 8 aa, Artificial Sequence
  Light chain CDR2
DKSNRPSG

SEQ ID NO: 6 - protein, 9 aa, Artificial Sequence
  Light chain CDR3
QSYANTLSL

SEQ ID NO: 7 - protein, 118 aa, Artificial Sequence
  Heavy chain variable region
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSS

SEQ ID NO: 8 - protein, 108 aa, Artificial Sequence
  Light chain variable region
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLG

SEQ ID NO: 9 - protein, 463 aa, Artificial Sequence
  Heavy chain amino acid sequence
  SIGNAL (1)..(19)
MKHLWFFLLLVAAPRWVLSEVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 10 - protein, 232 aa, Artificial Sequence
  Light chain amino acid sequence
  SIGNAL (1)..(19)
MAWALLLLTLLTQGTGSWADIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 444 aa, Artificial Sequence
  Heavy chain amino acid sequence without predicted signal sequence
EVQLVESGGGLVQPGGSLRLSCAASGFTFSHYTLSWVRQAPGKGLEWVSVISGDGSYTYYADSVKGRFTISSDNSKNTLYLQMNSLRAEDTAVYYCARNFIKYVFANWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 12 - protein, 213 aa, Artificial Sequence
  Light chain amino acid sequence without predicted signal sequence
DIELTQPPSVSVAPGQTARISCSGDNIGSFYVHWYQQKPGQAPVLVIYDKSNRPSGIPERFSGSNSGNTATLTISGTQAEDEADYYCQSYANTLSLVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 13 - protein, 151 aa, Artificial Sequence
  Human FZD1 Fri domain
QQPPPPPQQQQSGQQYNGERGISVPDHGYCQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELCVGQNTSDKGT

SEQ ID NO: 14 - protein, 136 aa, Artificial Sequence
  Human FZD2 Fri domain
QFHGEKGISIPDHGFCQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQICVGQNHSEDG

SEQ ID NO: 15 - protein, 121 aa, Artificial Sequence
  Human FZD3 Fri domain
HSLFSCEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDCDEPYPRLVDL

SEQ ID NO: 16 - protein, 131 aa, Artificial Sequence
  Human FZD4 Fri domain
FGDEEERRCDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMCMEGPGDEEV

SEQ ID NO: 17 - protein, 131 aa, Artificial Sequence
  Human FZD5 Fri domain
ASKAPVCQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLCMDYNRSEATT

SEQ ID NO: 18 - protein, 127 aa, Artificial Sequence
  Human FZD6 Fri domain
HSLFTCEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYCDETVPVTFDPHTEFLG

SEQ ID NO: 19 - protein, 138 aa, Artificial Sequence
  Human FZD7 Fri domain
QPYHGEKGISVPDHGFCQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEICVGQNTSDGSG

SEQ ID NO: 20 - protein, 131 aa, Artificial Sequence
  Human FZD8 Fri domain
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTT

SEQ ID NO: 21 - protein, 137 aa, Artificial Sequence
  Human FZD9 Fri domain
LEIGRFDPERGRGAAPCQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALCMEAPENA

SEQ ID NO: 22 - protein, 134 aa, Artificial Sequence
  Human FZD10 Fri domain
ISSMDMERPGDGKCQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLCMEAPNNG

SEQ ID NO: 23 - protein, 112 aa, Artificial Sequence
  Human FZD1 amino acids 116-227
CQPISIPLCTDIAYNQTIMPNLLGHTNQEDAGLEVHQFYPLVKVQCSAELKFFLCSMYAPVCTVLEQALPPCRSLCERARQGCEALMNKFGFQWPDTLKCEKFPVHGAGELC

SEQ ID NO: 24 - protein, 112 aa, Artificial Sequence
  Human FZD2 amino acids 39-150
CQPISIPLCTDIAYNQTIMPNLLGHTNQEDAGLEVHQFYPLVKVQCSPELRFFLCSMYAPVCTVLEQAIPPCRSICERARQGCEALMNKFGFQWPERLRCEHFPRHGAEQIC

SEQ ID NO: 25 - protein, 106 aa, Artificial Sequence
  Human FZD3 amino acids 28-133
CEPITLRMCQDLPYNTTFMPNLLNHYDQQTAALAMEPFHPMVNLDCSRDFRPFLCALYAPICMEYGRVTLPCRRLCQRAYSECSKLMEMFGVPWPEDMECSRFPDC

SEQ ID NO: 26 - protein, 114 aa, Artificial Sequence
  Human FZD4 amino acids 48-161
CDPIRISMCQNLGYNVTKMPNLVGHELQTDAELQLTTFTPLIQYGCSSQLQFFLCSVYVPMCTEKINIPIGPCGGMCLSVKRRCEPVLKEFGFAWPESLNCSKFPPQNDHNHMC

SEQ ID NO: 27 - protein, 115 aa, Artificial Sequence
  Human FZD5 amino acids 33-147
CQEITVPMCRGIGYNLTHMPNQFNHDTQDEAGLEVHQFWPLVEIQCSPDLRFFLCSMYTPICLPDYHKPLPPCRSVCERAKAGCSPLMRQYGFAWPERMSCDRLPVLGRDAEVLC

SEQ ID NO: 28 - protein, 106 aa, Artificial Sequence
  Human FZD6 amino acids 24-129
CEPITVPRCMKMAYNMTFFPNLMGHYDQSIAAVEMEHFLPLANLECSPNIETFLCKAFVPTCIEQIHVVPPCRKLCEKVYSDCKKLIDTFGIRWPEELECDRLQYC

SEQ ID NO: 29 - protein, 112 aa, Artificial Sequence
  Human FZD7 amino acids 49-160
CQPISIPLCTDIAYNQTILPNLLGHTNQEDAGLEVHQFYPLVKVQCSPELRFFLCSMYAPVCTVLDQAIPPCRSLCERARQGCEALMNKFGFQWPERLRCENFPVHGAGEIC

SEQ ID NO: 30 - protein, 114 aa, Artificial Sequence
  Human FZD8 amino acids 35-148
CQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLC

SEQ ID NO: 31 - protein, 114 aa, Artificial Sequence
  Human FZD9 amino acids 39-152
CQAVEIPMCRGIGYNLTRMPNLLGHTSQGEAAAELAEFAPLVQYGCHSHLRFFLCSLYAPMCTDQVSTPIPACRPMCEQARLRCAPIMEQFNFGWPDSLDCARLPTRNDPHALC

SEQ ID NO: 32 - protein, 114 aa, Artificial Sequence
  Human FZD10 amino acids 34-147
CQPIEIPMCKDIGYNMTRMPNLMGHENQREAAIQLHEFAPLVEYGCHGHLRFFLCSLYAPMCTEQVSTPIPACRVMCEQARLKCSPIMEQFNFKWPDSLDCRKLPNKNDPNYLC

SEQ ID NO: 33 - protein, 129 aa, Artificial Sequence
  Human FZD8 Fri domain- variant
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDL

SEQ ID NO: 34 - protein, 227 aa, Artificial Sequence
  Human IgG1 Fc region
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 35 - protein, 227 aa, Artificial Sequence
  Human IgG1 Fc region- variant
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 36 - protein, 230 aa, Artificial Sequence
  Human IgG1 Fc region
KSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 232 aa, Artificial Sequence
  Human IgG1 Fc region
EPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 224 aa, Artificial Sequence
  Human IgG2 Fc region
CVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 39 - protein, 360 aa, Artificial Sequence
  FZD8-Fc variant 54F03
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 40 - protein, 360 aa, Artificial Sequence
  FZD8-Fc variant
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 360 aa, Artificial Sequence
  FZD8-Fc variant
ASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLS

SEQ ID NO: 42 - protein, 388 aa, Artificial Sequence
  FZD8-Fc variant 54F03
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTGRADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 388 aa, Artificial Sequence
  FZD8-Fc variant 54F16
MEWGYLLEVTSLLAALALLQRSSGAAAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 390 aa, Artificial Sequence
  FZD8-Fc variant 54F26
MEWGYLLEVTSLLAALFLLQRSPIVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 45 - protein, 390 aa, Artificial Sequence
  FZD8-Fc variant 54F28
MEWGYLLEVTSLLAALLLLQRSPFVHAASAKELACQEITVPLCKGIGYNYTYMPNQFNHDTQDEAGLEVHQFWPLVEIQCSPDLKFFLCSMYTPICLEDYKKPLPPCRSVCERAKAGCAPLMRQYGFAWPDRMRCDRLPEQGNPDTLCMDYNRTDLTTEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 46 - protein, 83 aa, Artificial Sequence
  Human Wnt1 C-terminal cysteine rich domain
DLVYFEKSPNFCTYSGRLGTAGTAGRACNSSSPALDGCELLCCGRGHRTRTQRVTERCNCTFHWCCHVSCRNCTHTRVLHECL

SEQ ID NO: 47 - protein, 94 aa, Artificial Sequence
  Human Wnt2 C-terminal cysteine rich domain
DLVYFENSPDYCIRDREAGSLGTAGRVCNLTSRGMDSCEVMCCGRGYDTSHVTRMTKCGCKFHWCCAVRCQDCLEALDVHTCKAPKNADWTTAT

SEQ ID NO: 48 - protein, 94 aa, Artificial Sequence
  Human Wnt2b C-terminal cysteine rich domain
DLVYFDNSPDYCVLDKAAGSLGTAGRVCSKTSKGTDGCEIMCCGRGYDTTRVTRVTQCECKFHWCCAVRCKECRNTVDVHTCKAPKKAEWLDQT

SEQ ID NO: 49 - protein, 83 aa, Artificial Sequence
  Human Wnt3 C-terminal cysteine rich domain
DLVYYENSPNFCEPNPETGSFGTRDRTCNVTSHGIDGCDLLCCGRGHNTRTEKRKEKCHCIFHWCCYVSCQECIRIYDVHTCK

SEQ ID NO: 50 - protein, 83 aa, Artificial Sequence
  Human Wnt3a C-terminal cysteine rich domain
DLVYYEASPNFCEPNPETGSFGTRDRTCNVSSHGIDGCDLLCCGRGHNARAERRREKCRCVFHWCCYVSCQECTRVYDVHTCK

SEQ ID NO: 51 - protein, 83 aa, Artificial Sequence
  Human Wnt7a C-terminal cysteine rich domain
DLVYIEKSPNYCEEDPVTGSVGTQGRACNKTAPQASGCDLMCCGRGYNTHQYARVWQCNCKFHWCCYVKCNTCSERTEMYTCK

SEQ ID NO: 52 - protein, 83 aa, Artificial Sequence
  Human Wnt7b C-terminal cysteine rich domain
DLVYIEKSPNYCEEDAATGSVGTQGRLCNRTSPGADGCDTMCCGRGYNTHQYTKVWQCNCKFHWCCFVKCNTCSERTEVFTCK

SEQ ID NO: 53 - protein, 108 aa, Artificial Sequence
  Human Wnt8a C-terminal cysteine rich domain
ELIFLEESPDYCTCNSSLGIYGTEGRECLQNSHNTSRWERRSCGRLCTECGLQVEERKTEVISSCNCKFQWCCTVKCDQCRHVVSKYYCARSPGSAQSLGRVWFGVYI

SEQ ID NO: 54 - protein, 107 aa, Artificial Sequence
  Human Wnt8b C-terminal cysteine rich domain
ELVHLEDSPDYCLENKTLGLLGTEGRECLRRGRALGRWELRSCRRLCGDCGLAVEERRAETVSSCNCKFHWCCAVRCEQCRRRVTKYFCSRAERPRGGAAHKPGRKP

SEQ ID NO: 55 - protein, 83 aa, Artificial Sequence
  Human Wnt10a C-terminal cysteine rich domain
DLVYFEKSPDFCEREPRLDSAGTVGRLCNKSSAGSDGCGSMCCGRGHNILRQTRSERCHCRFHWCCFVVCEECRITEWVSVCK

SEQ ID NO: 56 - protein, 83 aa, Artificial Sequence
  Human Wnt10b C-terminal cysteine rich domain
ELVYFEKSPDFCERDPTMGSPGTRGRACNKTSRLLDGCGSLCCGRGHNVLRQTRVERCHCRFHWCCYVLCDECKVTEWVNVCK

SEQ ID NO: 57 - protein, 8 aa, Artificial Sequence
  Linker
ESGGGGVT

SEQ ID NO: 58 - protein, 9 aa, Artificial Sequence
  Linker
LESGGGGVT

SEQ ID NO: 59 - protein, 6 aa, Artificial Sequence
  Linker
GRAQVT

SEQ ID NO: 60 - protein, 6 aa, Artificial Sequence
  Linker
WRAQVT

SEQ ID NO: 61 - protein, 8 aa, Artificial Sequence
  Linker
ARGRAQVT